(19) United States
(12) Patent Application Publication
(10) Pub. No.: US 2009/0117112 A1
(43) Pub. Date: May 7, 2009

(54) IMMUNOMODULATORY COMPOSITIONS AND USES THEREFOR

(75) Inventors: Craig A. Smith, Seattle, WA (US); Steven Wiley, Seattle, WA (US); Ajamete Kaykas, Seattle, WA (US); Jalal Vakili, Seattle, WA (US); Peter Probst, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: VIRAL LOGIC SYSTEMS TECHNOLOGY CORPORATION, SEATTLE, WA (US)

(21) Appl. No.: 12/246,291

(22) Filed: Oct. 6, 2008

Related U.S. Application Data
(63) Continuation of application No. 11/541,449, filed on Sep. 29, 2006.
(60) Provisional application No. 60/721,876, filed on Sep. 29, 2005.
(60) Provisional application No. 60/784,710, filed on Mar. 22, 2006.
(60) Provisional application No. 60/801,992, filed on May 19, 2006.

(51) Int. Cl. A61K 39/395; C07K 16/00; A61P 37/02

(52) U.S. Cl. 424/136.1; 530/389.1; 530/388.22; 530/387.3; 530/391.1; 424/130.1

(57) ABSTRACT

The poxvirus proteins designated A41L and 130L bind to three receptor-like protein tyrosine phosphatases (RPTP), leukocyte common antigen related protein (LAR), RPTP-δ, and RPTP-σ, that are present on the cell surface of immune cells. When a host is infected with the poxvirus, binding of A41L to cell surface proteins on the host cells results in suppression of the immune response. The present invention provides agents such as antibodies, and antigen-binding fragments thereof, small molecules, aptamers, small interfering RNAs, and peptide-IgFc fusion polypeptides that interact with one or more of LAR, RPTP-δ, and RPTP-σ expressed by immune cells or interact with a polynucleotide encoding the RPTP. Also provided are RPTP Ig domain oligomers and Fc fusion polypeptides. Such agents are useful for treating an immunological disorder in a subject according to the methods described herein.

### CROSS-REFERENCE TO RELATED APPLICATIONS

This application is a continuation of pending U.S. patent application Ser. No. 11/541,449 filed Sep. 29, 2006, which claims the benefit of U.S. Provisional Application No. 60/721,876 filed Sep. 29, 2005; U.S. Provisional Application No. 60/784,710 filed Mar. 22, 2006; and U.S. Provisional Application No. 60/801,992 filed May 19, 2006, all of which non-provisional and provisional applications are incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930118_{—}401C1_SEQUENCE_LISTING.txt. The text file is 755 KB, was created on Oct. 6, 2008, and is being submitted electronically via EFS-Web.

### BACKGROUND OF THE INVENTION

1. Field of the Invention

The present invention provides agents that affect the function of one or more of three receptor-like protein tyrosine phosphatases (RPTP), leukocyte common antigen related protein (LAR), RPTP-δ, and RPTP-σ, present on the cell surface of immune cells, in the same or in a similar manner as poxvirus proteins, such as A41L and 130L. Such agents are useful for altering immunoresponsiveness of an immune cell and for treating immunological disorders in a subject.

2. Description of the Related Art

Poxviruses form a group of double-stranded DNA viruses that replicate in the cytoplasm of a cell and have adapted to replicate in numerous different hosts. One adaptive mechanism of many poxviruses involves the acquisition of host genes that allow the viruses to evade the host's immune system and/or facilitate viral replication (Bugert and Darai, Virus Genes 21:111 (2000); Alcami et al., Semin. Virol. 8:419 (1998); McFadden and Barry, Semin. Virol. 8:429 (1998)). This process is facilitated by the relatively large size and complexity of the poxvirus genome. Vaccinia virus, a prototype poxvirus widely used as a smallpox vaccine, has a genome of approximately 190 kilobases, which could potentially encode more than 200 proteins (Goebel et al., Virology 179:247 (1990)). Even though the entire genome of vaccinia virus has been sequenced, the function of many of the potential open reading frames (ORFs), and the existence of polypeptides encoded thereby, remains unknown.

Certain poxvirus polypeptides contribute to the virulence of the virus. An ORF designated A41L is present in several different poxviruses, including Cowpox virus (CPV), vaccinia virus (strains Copenhagen, Ankara, Tian Tan and WR) and variola virus (including strains Harvey, India-1967 and Garcia-1966). The A41L gene encodes a glycoprotein (herein called A41L polypeptide) that is a viral virulence factor, which is secreted by cells infected with a poxvirus (see, e.g., U.S. Pat. No. 6,852,486; International Patent Application Publication WO 98/37217; Ng et al., J. Gen. Virol. 82:2095-105 (2001)). A41L acts, at least in part, in a host infected with a poxvirus to suppress an immune response specific for the virus.

Identification of additional viral virulence factors and identification of cell polypeptides that are expressed by immune cells and that interact with A41L would be useful and beneficial for treating immunological disorders, such as, for example, inflammatory diseases and autoimmune diseases, including multiple sclerosis, rheumatoid arthritis, and systemic lupus erythematosus (SLE). A need exists to identify and develop compositions that can be used for treatment and prophylaxis of such immunological diseases and disorders.

### BRIEF SUMMARY OF THE INVENTION

The several embodiments described herein relate to compositions and methods for preventing and treating immunological diseases and disorders. In one embodiment, an isolated antibody, or antigen-binding fragment thereof, is provided (a) that specifically binds to at least two receptor-like protein tyrosine phosphatase (RPTP) polypeptides selected from (i) leukocyte common antigen-related protein (LAR); (ii) RPTP-σ; and (iii) RPTP-δ; and (b) that competitively inhibits binding of a poxvirus polypeptide to the at least two RPTP polypeptides. In another embodiment, an isolated antibody, or antigen-binding fragment thereof, specifically binds to at least one receptor-like protein tyrosine phosphatase (RPTP) present on the cell surface of an immune cell, wherein the at least one RPTP is RPTP-σ or RPTP-δ, and wherein binding of the antibody, or antigen-binding fragment thereof, to the RPTP that is present on the cell surface of the immune cell suppresses immunoresponsiveness of the immune cell. In a specific embodiment, the antibody is a polyclonal antibody or a monoclonal antibody. In other certain specific embodiments, the antigen-binding fragment is selected from F(ab′)₂, Fab′, Fab, Fd, Fv, and single chain Fv (scFv). In another embodiment, the poxvirus polypeptide is either A41L or Yaba-Like Disease Virus 130L. Further provided herein is a composition that comprises any of the antibodies, or antigen binding fragments thereof, and a pharmaceutically suitable excipient. Also provided in another embodiment, is a method of suppressing an immune response in a subject comprising administering to the subject the composition. In still another embodiment, is a method for treating an immunological disease or disorder in a subject comprising administering to the subject the composition. In another embodiment is provided a method of manufacture for producing the composition.

Also provided herein is a bispecific antibody comprising (a) a first antigen-binding moiety that is capable of specifically binding to a receptor-like protein tyrosine phosphatase (RPTP), wherein the RPTP is selected from (i) leukocyte common antigen-related protein (LAR); (ii) RPTP-σ; and (iii) RPTP-δ; and (b) a second antigen-binding moiety that is capable of specifically binding to a RPTP, wherein the RPTP is selected from (i) leukocyte common antigen-related protein (LAR); (ii) RPTP-σ; and (iii) RPTP-δ, wherein the first antigen-binding moiety and the second antigen-binding moiety are different, and wherein the bispecific antibody suppresses immunoresponsiveness of an immune cell. Also provided is a composition comprising the bispecific antibody and a pharmaceutically suitable excipient. Also provided in another embodiment, is a method of suppressing an immune response in a subject comprising administering to the subject the composition. In still another embodiment, is a method for treating an immunological disease or disorder in a subject comprising administering to the subject the composition. Also provided in yet another embodiment is a method of manufacture for producing the bispecific antibody.

In another embodiment, a fusion polypeptide is provided that comprises (a) an immunoglobulin-like domain 2 polypeptide of a first receptor-like protein tyrosine phosphatase (RPTP); (b) an immunoglobulin-like domain 3 polypeptide of a second RPTP; and (c) an immunoglobulin Fc polypeptide or mutein thereof, wherein each of the first RPTP and the second RPTP is selected from (i) leukocyte common antigen-related protein (LAR); (ii) RPTP-σ; and (iii) RPTP-δ, and wherein the first and second RPTP are the same or different. In one particular embodiment, the first RPTP and the second RPTP are the same. In another specific embodiment, the first RPTP is RPTP-σ and the second RPTP is RPTP-σ, and wherein the fusion polypeptide further comprises an immunoglobulin-like domain 1 polypeptide of RPTP-σ. In yet another embodiment, the first RPTP is RPTP-δ and the second RPTP is RPTP-δ, wherein the fusion polypeptide further comprises an immunoglobulin-like domain 1 polypeptide of RPTP-δ. Also provided is a composition that comprises the fusion polypeptide and a pharmaceutically suitable excipient. Also provided in another embodiment, is a method of suppressing an immune response in a subject comprising administering to the subject the composition. In still another embodiment, is a method for treating an immunological disease or disorder in a subject comprising administering to the subject the composition. In another embodiment is provided a method of manufacture for producing the fusion polypeptide.

Also provided herein is a composition comprising (a) at least one immunoglobulin-like domain 2 polypeptide of a first receptor-like protein tyrosine phosphatase (RPTP) and (b) at least one immunoglobulin-like domain 3 polypeptide of a second RPTP, wherein the first and second RPTP are the same or different and selected from (i) leukocyte common antigen-related protein (LAR); (ii) RPTP-σ; and (iii) RPTP-δ. In a specific embodiment, the first RPTP and the second RPTP are the same, and in another specific embodiment, the first RPTP and the second RPTP are different. In one specific embodiment, the first RPTP is RPTP-σ and the second RPTP is RPTP-σ, and the composition further comprises an immunoglobulin-like domain 1 polypeptide of RPTP-σ. In yet another specific embodiment, the first RPTP is RPTP-δ and the second RPTP is RPTP-δ, and the composition further comprises an immunoglobulin-like domain 1 polypeptide of RPTP-δ.

Also provided is a composition comprising a polypeptide dimer wherein the dimer comprises (a) a first monomer comprising an immunoglobulin-like domain 2 polypeptide and an immunoglobulin-like domain 3 polypeptide of a first receptor-like protein tyrosine phosphatase (RPTP); and (b) a second monomer comprising an immunoglobulin-like domain 2 polypeptide and an immunoglobulin-like domain 3 polypeptide of a second RPTP, wherein the first and second RPTP are the same or different and selected from (i) leukocyte common antigen-related protein (LAR); (ii) RPTP-σ; and (iii) RPTP-δ. In one particular embodiment, the first RPTP and the second RPTP are different. In another particular embodiment, the first RPTP and the second RPTP are the same. In a specific embodiment, the first monomer further comprises an immunoglobulin-like domain 1 of the first RPTP, and the second monomer further comprises an immunoglobulin-like domain 1 of the second RPTP. In another specific embodiment, the first monomer is fused to an immunoglobulin Fc polypeptide, and the second monomer is fused to an immunoglobulin Fc polypeptide.

In other specific embodiments, each of the compositions described herein further comprises a pharmaceutically suitable excipient. Also provided in another embodiment, is a method of suppressing an immune response in a subject comprising administering to the subject the composition. In still another embodiment, is a method for treating an immunological disease or disorder in a subject comprising administering to the subject the composition. In another embodiment is provided a method of manufacture for producing the composition.

In another embodiment, fusion polypeptide is provided that comprises a poxvirus polypeptide fused with a mutein Fc polypeptide, wherein the mutein Fc polypeptide comprises the amino acid sequence of the Fc portion of a human IgG1 immunoglobulin comprising at least one mutation, wherein the at least one mutation is a substitution or a deletion of a cysteine residue in the hinge region, wherein the substituted or deleted cysteine residue is the cysteine residue most proximal to the amino terminus of the hinge region of a wildtype human IgG1 immunoglobulin Fc portion, and wherein the poxvirus polypeptide is capable of binding to a receptor-like protein tyrosine phosphatase (RPTP) selected from (i) leukocyte common antigen-related protein (LAR); (ii) RPTP-σ; and (iii) RPTP-δ. In one particular embodiment, the mutein Fc polypeptide comprises at least one second mutation, wherein the at least one second mutation is a substitution of at least one amino acid in the CH2 domain such that the capability of the fusion polypeptide to bind to an IgG Fc receptor is reduced.

Also provided herein is a composition comprising any one of the fusion polypeptides and further comprising a pharmaceutically suitable excipient. Compositions are also provided comprising (a) the antibody or antigen-binding fragment thereof, described above, and (b) a pharmaceutically suitable excipient. Also provided in another embodiment, is a method of suppressing an immune response in a subject comprising administering to the subject the composition. In still another embodiment, is a method for treating an immunological disease or disorder in a subject comprising administering to the subject the composition. In another embodiment is provided a method of manufacture for producing the fusion polypeptide.

In one embodiment, is provided an isolated antibody, or antigen-binding fragment thereof, that specifically binds to at least two receptor-like protein tyrosine phosphatase (RPTP) polypeptides selected from leukocyte common antigen-related protein (LAR); (ii) RPTP-σ; and (iii) RPTP-δ; and (b) competitively inhibits binding of A41L to the at least two RPTP polypeptides. In particular embodiments, the antibody specifically binds LAR and RPTP-σ; the antibody specifically binds LAR and RPTP-δ; or the antibody specifically binds RPTP-σ and RPTP-δ. In another particular embodiment, the antibody specifically binds LAR, RPTP-σ, and RPTP-δ.

In another embodiment, an isolated antibody, or antigen-binding fragment thereof, is provided that specifically binds to either receptor-like protein tyrosine phosphatase-sigma (RPTP-σ) or receptor-like protein tyrosine phosphatase-delta (RPTP-δ) or both, wherein binding of the antibody, or antigen-binding fragment thereof, inhibits binding of A41L to RPTP-σ, RPTP-δ, or both.

In yet another embodiment, is provided an isolated antibody, or antigen-binding fragment thereof, that (a) specifically binds to at least two receptor-like protein tyrosine phosphatase (RPTP) polypeptides selected from leukocyte common antigen-related protein (LAR); (ii) RPTP-σ; and (iii) RPTP-δ; and (b) suppresses immunoresponsiveness of an immune cell that expresses at least one of the RPTP polypeptides. In particular embodiments, the antibody specifically binds LAR and RPTP-σ; the antibody specifically binds LAR and RPTP-δ; or the antibody specifically binds RPTP-σ and RPTP-δ. In another particular embodiment, the antibody specifically binds LAR, RPTP-σ, and RPTP-δ.

In still yet another embodiment, an isolated antibody, or antigen-binding fragment thereof, (a) specifically binds to at least two receptor-like protein tyrosine phosphatases (RPTP) polypeptides selected from (i) leukocyte common antigen-related protein (LAR); (ii) RPTP-σ; and (iii) RPTP-δ; and (b) inhibits binding of A41L to an immune cell that expresses at least one of LAR; (ii) RPTP-σ; and (iii) RPTP-δ. In particular embodiments, the antibody specifically binds LAR and RPTP-σ; the antibody specifically binds LAR and RPTP-δ; or the antibody specifically binds RPTP-σ and RPTP-δ. In another particular embodiment, the antibody specifically binds LAR, RPTP-σ, and RPTP-δ.

In one embodiment, an isolated antibody, or antigen-binding fragment thereof, is provided that specifically binds to receptor-like protein tyrosine phosphatase-sigma (RPTP-σ), wherein binding of the antibody, or antigen-binding fragment thereof, to RPTP-σ that is present on the cell surface of an immune cell suppresses immunoresponsiveness of the immune cell. In another embodiment, is provided an isolated antibody, or antigen-binding fragment thereof, that specifically binds to receptor-like protein tyrosine phosphatase-delta (RPTP-δ), wherein binding of the antibody, or antigen-binding fragment thereof, to RPTP-δ that is present on the cell surface of an immune suppresses immunoresponsiveness of the immune cell that expresses RPTP-δ. In yet another embodiment, an isolated antibody, or antigen-binding fragment thereof, is provided that specifically binds to either receptor-like protein tyrosine phosphatase-sigma (RPTP-σ) or receptor-like protein tyrosine phosphatase-delta (RPTP-δ) or to both RPTP-σ and RPTP-δ, wherein binding of the antibody, or antigen-binding fragment thereof, with either RPTP-σ or RPTP-δ or to both RPTP-σ and RPTP-δ that are present on the cell surface of an immune cell suppresses immunoresponsiveness of the immune cell.

In certain embodiments, with respect to any one of the above-described antibodies, the antibody is a polyclonal antibody. In other certain embodiments, the antibody is a monoclonal antibody. In another specific embodiment, the monoclonal antibody is selected from a mouse monoclonal antibody, a human monoclonal antibody, a rat monoclonal antibody, and a hamster monoclonal antibody. Also provided herein is host cell that expresses the monoclonal antibody; and in certain specific embodiments, the host cell is a hybridoma cell. In another particular embodiment, the antibody is a humanized antibody or a chimeric antibody. In another embodiment, a host cell is provided that expresses the humanized antibody or a chimeric antibody.

In another particular embodiment, a composition is provided that comprises any one of the above-described antibodies (or antigen-binding fragment thereof) and a pharmaceutically suitable carrier. Also provided in another embodiment is a method of manufacture for producing any of the aforementioned antibodies, or antigen-binding fragments thereof.

In other specific embodiments, with respect to any one of the antigen-binding fragments of any one of the above-described antibodies, the antigen-binding fragment is selected from F(ab′)₂, Fab′, Fab, Fd, and Fv. In another specific embodiment, the antigen-binding fragment is of human, mouse, chicken, or rabbit origin. In still another specific embodiment, the antigen-binding fragment is a single chain Fv (scFv). In another particular embodiment, a composition is provided that comprises any one of the antigen-binding fragments of any one of the above-described antibodies and a pharmaceutically suitable carrier.

Also provided in another embodiment is an isolated antibody comprising an anti-idiotype antibody, or antigen-binding fragment thereof, that specifically binds to any one of the aforementioned antibodies, or to an antigen binding fragment thereof. In certain embodiments, the anti-idiotype antibody is a polyclonal antibody. In other certain embodiments, the anti-idiotype antibody is a monoclonal antibody. Also provided herein is a host cell that expresses the anti-idiotype antibody. In certain specific embodiments, the host cell is a hybridoma cell. In another particular embodiment, a composition is provided that comprises the anti-idiotype antibody, or antigen-binding fragment thereof, and a pharmaceutically suitable carrier.

In one embodiment, also provided is a bispecific antibody comprising (a) a first antigen-binding moiety that is capable of specifically binding to a RPTP, wherein the RPTP is selected from (i) leukocyte common antigen-related protein (LAR); (ii) RPTP-σ; and (iii) RPTP-δ; and (b) a second antigen-binding moiety that is capable of specifically binding to a RPTP, wherein the RPTP selected from (i) leukocyte common antigen-related protein (LAR); (ii) RPTP-σ; and (iii) RPTP-δ, wherein the bispecific antibody suppresses immunoresponsiveness of an immune cell. In a specific embodiment, the first antigen-binding moiety is capable of specifically binding to LAR and the second antigen-binding moiety is capable of specifically binding to RPTP-σ. In another specific embodiment, the first antigen-binding moiety is capable of specifically binding to LAR and the second antigen-binding moiety is capable of specifically binding to RPTP-δ. In yet another specific embodiment, the first antigen-binding moiety is capable of specifically binding to RPTP-σ and the second antigen-binding moiety is capable of specifically binding to RPTP-δ. In another particular embodiment, a composition is provided that comprises the bispecific antibody and a pharmaceutically suitable carrier.

In another embodiment, a fusion polypeptide is provided that comprises at least one immunoglobulin-like domain of a RPTP selected from (i) leukocyte common antigen-related protein (LAR); (ii) RPTP-σ; and (iii) RPTP-δ, fused with at least one immunoglobulin constant region domain. In a specific embodiment, the at least one immunoglobulin-like domain of the RPTP is fused with an immunoglobulin Fc polypeptide. In a particular embodiment, the Fc polypeptide is derived from a human IgG1 immunoglobulin. In another specific embodiment, the RPTP is LAR and the fusion polypeptide suppresses immunoresponsiveness of an immune cell. In a specific embodiment, the Fc polypeptide is a mutein Fc polypeptide that comprises a substitution or a deletion of a cysteine residue in the hinge region, and wherein the substituted or deleted cysteine residue is the cysteine residue most proximal to the amino terminus of the hinge region of the Fc portion of a wildtype IgG1 immunoglobulin. In yet another specific embodiment, the Fc polypeptide is a mutein Fc polypeptide that comprises at least one substitution of an amino acid residue in the CH2 domain of the mutein Fc polypeptide, such that the capability of the fusion polypeptide to bind to an IgG Fc receptor is reduced. In still yet another specific embodiment, the mutein Fc polypeptide further comprises a substitution or a deletion of a cysteine residue in the hinge region, wherein the substituted or deleted cysteine residue is the cysteine residue most proximal to the amino terminus of the hinge region of the Fc portion of a wildtype IgG1 immunoglobulin. In yet another specific embodiment, the RPTP is RPTP-σ, and the fusion polypeptide suppresses immunoresponsiveness of an immune cell. In another specific embodiment, the RPTP is RPTP-δ, and wherein the fusion polypeptide suppresses immunoresponsiveness of an immune cell. In another particular embodiment, a composition is provided that comprises the fusion polypeptide and a pharmaceutically suitable carrier.

In one embodiment, an agent is provided that specifically binds to at least two receptor-like protein tyrosine phosphatase (RPTP) polypeptides selected from leukocyte common antigen-related protein (LAR); (ii) RPTP-σ; and (iii) RPTP-δ; and (b) impairs binding of A41L to any one of LAR, RPTP-σ, and RPTP-δ. In a certain embodiment, the agent impairs binding of A41L to any one of LAR, RPTP-σ, and RPTP-δ present on the cell surface of an immune cell. In other specific embodiments, the agent is selected from an antibody or antigen binding fragment thereof, a small molecule; an aptamer; and a peptide-IgFc fusion polypeptide. In another particular embodiment, a composition is provided that comprises the agent and a pharmaceutically suitable carrier.

Also provided in an embodiment is agent that specifically impairs expression of at least two receptor-like protein tyrosine phosphatase (RPTP) polypeptides selected from leukocyte common antigen-related protein (LAR); (ii) RPTP-σ; and (iii) RPTP-δ. In a particular embodiment, the agent comprises an antisense polynucleotide, and in another particular embodiment, the agent comprises a short interfering RNA (siRNA). In another particular embodiment, a composition is provided that comprises the agent and a pharmaceutically suitable carrier.

In another embodiment, a method is provided for identifying an agent that suppresses immunoresponsiveness of an immune cell comprising: (a) contacting (1) a candidate agent; (2) an immune cell that expresses at least one receptor-like protein tyrosine phosphatase (RPTP) polypeptide selected from (i) leukocyte common antigen-related protein (LAR); (ii) RPTP-σ; and (iii) RPTP-δ; and (3) A41L, under conditions and for a time sufficient to permit interaction between the at least one RPTP polypeptide and A41L; and (b) determining a level of binding of A41L to the immune cell in the presence of the candidate agent and comparing a level of binding of A41L to the immune cell in the absence of the candidate agent, wherein a decrease in the level of binding of A41L to the immune cell in the presence of the candidate agent indicates that the candidate agent suppresses immunoresponsiveness of the immune cell. In a specific embodiment, the immune cell expresses at least two RPTP polypeptides selected from (i) LAR; (ii) RPTP-σ; and (iii) RPTP-δ.

Also provided herein is a method for identifying an agent that inhibits binding of A41L to at least two receptor-like protein tyrosine phosphatase (RPTP) polypeptides comprising: (a) contacting (1) a candidate agent; (2) a biological sample comprising at least two RPTP polypeptides selected from (i) leukocyte common antigen-related protein (LAR); (ii) RPTP-σ; and (iii) RPTP-δ; and (3) A41L, under conditions and for a time sufficient to permit interaction between the at least two RPTP polypeptides and A41L; and (b) determining a level of binding of A41L to the at least two RPTP polypeptides in the presence of the candidate agent and comparing a level of binding of A41L to the at least two RPTP polypeptides in the absence of the candidate agent, wherein a decrease in the level of binding of A41L to the at least two RPTP polypeptides in the presence of the candidate agent indicates that the candidate agent inhibits binding of A41L to the at least two RPTP polypeptides.

In another embodiment, a method is provided for suppressing an immune response in a subject comprising administering a composition that comprises a pharmaceutically suitable carrier and an antibody, or antigen-binding fragment thereof, that specifically binds to a receptor-like protein tyrosine phosphatase (RPTP)-σ. In one embodiment, method is provided for suppressing an immune response in a subject comprising administering a composition comprising a pharmaceutically suitable carrier and an antibody, or antigen-binding fragment thereof, that specifically binds to receptor-like protein tyrosine phosphatase (RPTP)-δ. In another embodiment, a method is provided for suppressing an immune response in a subject comprising administering a composition comprising a pharmaceutically suitable carrier and an antibody, or antigen-binding fragment thereof, that (a) specifically binds to at least two receptor-like protein tyrosine phosphatase (RPTP) polypeptides selected from (i) leukocyte common antigen-related protein (LAR); (ii) RPTP-σ; and (iii) RPTP-δ.

In one embodiment, a method is provided for treating an immunological disease or disorder in a subject comprising administering to the subject a pharmaceutically suitable carrier and an agent that either (a) alters a biological activity of at least one receptor-like protein tyrosine phosphatase (RPTP) polypeptide, wherein the RPTP is either RPTP-σ or RPTP-δ; or (b) alters a biological activity of at least two RPTP polypeptides selected from leukocyte common antigen-related protein (LAR); (ii) RPTP-σ; and (iii) RPTP-δ. In a specific embodiment, the immunological disease or disorder is an autoimmune disease or an inflammatory disease. In a certain embodiment, the autoimmune or inflammatory disease is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In another particular embodiment, the agent is selected from an antibody, or antigen-binding fragment thereof, a small molecule; an aptamer; an antisense polynucleotide; a small interfering RNA (siRNA); and a peptide-IgFc fusion polypeptide.

In one embodiment, is provided a method for treating a disease or disorder associated with alteration of at least one of cell migration, cell proliferation, and cell differentiation in a subject comprising administering to the subject a pharmaceutically suitable carrier and an agent that either (a) alters a biological activity of at least one of receptor-like protein tyrosine phosphatase (RPTP)-σ or RPTP-δ; or (b) alters a biological activity of at least two RPTP polypeptides selected from (i) leukocyte common antigen-related protein (LAR); (ii) RPTP-σ; and (iii) RPTP-δ. In certain embodiments, the disease or disorder is an immunological disease or disorder, a cardiovascular disease or disorder, a metabolic disease or disorder, or a proliferative disease or disorder. In a particular embodiment, the immunological disease or disorder is an autoimmune disease or an inflammatory disease. In another certain embodiment, the immunological disease or disorder is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In another particular embodiment, the cardiovascular disease or disorder is atherosclerosis, endocarditis, hypertension, or peripheral ischemic disease. In another particular embodiment, the agent is selected from an antibody, or antigen-binding fragment thereof, a small molecule; an aptamer; an antisense polynucleotide; a small interfering RNA (siRNA); and a peptide-IgFc fusion polypeptide.

In another embodiment, a method of manufacture is provided for producing an agent that suppresses immunoresponsiveness of an immune cell, comprising (a) identifying an agent that suppresses immunoresponsiveness of an immune cell, wherein the step of identifying comprises (1) contacting (i) a candidate agent; (ii) an immune cell that expresses at least one receptor-like protein tyrosine phosphatase (RPTP) polypeptide selected from leukocyte common antigen-related protein (LAR); RPTP-σ; and RPTP-δ; and (iii) A41L, under conditions and for a time sufficient to permit interaction between the at least one RPTP polypeptide and A41L; and (2) determining a level of binding of A41L to the immune cell in the presence of the candidate agent and comparing a level of binding of A41L to the immune cell in the absence of the candidate agent, wherein a decrease in the level binding of A41L to the immune cell in the presence of the candidate agent indicates that the candidate agent suppresses immunoresponsiveness of the immune cell; and (b) producing the agent identified in step (a). In certain embodiments, the agent is selected from an antibody, or antigen-binding fragment thereof, a small molecule; an aptamer; an antisense polynucleotide; a small interfering RNA (siRNA); and a peptide-IgFc fusion polypeptide. In another certain embodiment, the agent is an antibody, or antigen-binding fragment thereof.

In one embodiment, a fusion polypeptide comprises an A41L polypeptide fused in frame with a mutein Fc polypeptide, wherein the mutein Fc polypeptide comprises the amino acid sequence of the Fc portion of a human IgG1 immunoglobulin, wherein the mutein Fc polypeptide differs from the Fc portion of a wildtype human IgG1 immunoglobulin by comprising at least two mutations, wherein a first mutation in the mutein Fc polypeptide comprises substitution of at least one amino acid in the CH2 domain such that the capability of the fusion polypeptide to bind to an IgG Fc receptor is reduced, and wherein a second mutation in the mutein Fc polypeptide is a substitution or a deletion of a cysteine residue in the hinge region, wherein the cysteine residue is the cysteine residue most proximal to the amino terminus of the hinge region of a wildtype human IgG1 immunoglobulin. In a specific embodiment, the mutein Fc polypeptide comprises substitution of at least two amino acids in the CH2 domain. In another specific embodiment, the mutein Fc polypeptide comprises substitution of at least three amino acids in the CH2 domain. In yet another specific embodiment, the amino acid that is substituted in the CH2 domain is located at a position that corresponds to EU position number 235 in the CH2 domain of a human IgG immunoglobulin. In still another specific embodiment, a first amino acid that is substituted is located at a position that corresponds to EU position number 234 in the CH2 domain of a human IgG immunoglobulin and a second amino acid that is substituted is located at a position that corresponds to EU position number 235 in the CH2 domain of a human IgG immunoglobulin. In yet another specific embodiment, a first amino acid that is substituted is located at a position that corresponds to EU position number 234 in the CH2 domain of a human IgG immunoglobulin, a second amino acid that is substituted is located at a position that corresponds to EU position number 235 in the CH2 domain of a human IgG immunoglobulin, and a third amino acid that is substituted is located at a position that corresponds to EU position number 237 in the CH2 domain of a human IgG immunoglobulin. In a certain specific embodiment, the leucine reside located at a position that corresponds to EU position number 235 in the CH2 domain of a human IgG immunoglobulin is substituted with a glutamic acid residue or an alanine residue. In another particular embodiment, the leucine residue located at a position that corresponds to EU position number 234 in the CH2 domain of a human IgG immunoglobulin is substituted with an alanine residue. In still another specific embodiment, the glycine residue located at a position that corresponds to EU position number 237 in the CH2 domain of a human IgG immunoglobulin is substituted with an alanine residue. In another particular embodiment, the mutein Fc polypeptide further comprises substitution or deletion of at least one non-cysteine residue in the hinge region. In another particular embodiment, the mutein Fc polypeptide comprises a deletion of at least two amino acid residues in the hinge region, wherein the at least two amino acid residues include a cysteine residue and the adjacent C-terminal residue, wherein the cysteine residue is the cysteine residue most proximal to the amino terminus of the hinge region of a wildtype human IgG1 immunoglobulin. In a specific embodiment, the fusion polypeptide comprises the amino acid sequence set forth in SEQ ID NO:73.

Also provided herein is a method of suppressing an immune response in a subject comprising administering a composition that comprises a pharmaceutically suitable carrier and the fusion polypeptide comprising an A41L polypeptide fused in frame with a mutein Fc polypeptide described above. In a particular embodiment, the fusion polypeptide either (a) alters a biological activity of at least one of receptor-like protein tyrosine phosphatase (RPTP)-σ and RPTP-δ; or (b) alters a biological activity of at least two RPTP polypeptides selected from (i) leukocyte common antigen-related protein (LAR); (ii) RPTP-σ; and (iii) RPTP-δ.

In another embodiment, a method is provided for treating an immunological disease or disorder in a subject comprising administering to the subject a pharmaceutically suitable carrier and the fusion polypeptide comprising an A41L polypeptide fused in frame with a mutein Fc polypeptide described above. In a specific embodiment, the fusion polypeptide either (a) alters a biological activity of at least one of receptor-like protein tyrosine phosphatase (RPTP)-σ and RPTP-δ; or (b) alters a biological activity of at least two RPTP polypeptides selected from (i) leukocyte common antigen-related protein (LAR); (ii) RPTP-σ; and (iii) RPTP-δ. In another particular embodiment, the immunological disease or disorder is an autoimmune disease or an inflammatory disease, wherein in certain embodiments, the autoimmune or inflammatory disease is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis.

In one embodiment, a method is provided for treating a disease or disorder associated with alteration of at least one of cell migration, cell proliferation, and cell differentiation in a subject comprising administering to the subject a pharmaceutically suitable carrier and the fusion polypeptide comprising an A41L polypeptide fused in frame with a mutein Fc polypeptide described above. In a particular embodiment, the fusion polypeptide either (a) alters a biological activity of at least one of receptor-like protein tyrosine phosphatase (RPTP)-σ or RPTP-δ; or (b) alters a biological activity of at least two RPTP polypeptides selected from (i) leukocyte common antigen-related protein (LAR); (ii) RPTP-σ; and (iii) RPTP-δ. In another embodiment, the disease or disorder is an immunological disease or disorder, a cardiovascular disease or disorder, a metabolic disease or disorder, or a proliferative disease or disorder. In a specific embodiment, the immunological disease or disorder is an autoimmune disease or an inflammatory disease. In another specific embodiment, the immunological disease or disorder is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In yet another specific embodiment, the cardiovascular disease or disorder is atherosclerosis, endocarditis, hypertension, or peripheral ischemic disease. In another embodiment, is provided method of manufacture for producing the fusion polypeptide comprising an A41L polypeptide fused in frame with a mutein Fc polypeptide described above.

In another embodiment, an isolated antibody, or antigen-binding fragment thereof is provided that (a) specifically binds to at least one receptor-like protein tyrosine phosphatase (RPTP) polypeptide selected from (i) leukocyte common antigen-related protein (LAR); (ii) RPTP-σ; and (iii) RPTP-δ; and (b) competitively inhibits binding of a 130L polypeptide to the at least one RPTP polypeptide, wherein the amino acid sequence of the 130L polypeptide is at least 80% identical to the amino acid sequence set forth in SEQ ID NO:85 or SEQ ID NO:150. In a particular embodiment, the 130L polypeptide specifically binds to at least two RPTP polypeptides selected from (i) LAR; (ii) RPTP-σ; and (iii) RPTP-δ, and in another particular embodiment, the 130L polypeptide specifically binds to (i) LAR; (ii) RPTP-σ; and (iii) RPTP-δ. In certain specific embodiments, the antibody, or antigen-binding fragment thereof, specifically binds LAR and RPTP-σ. In another specific embodiment, the antibody, or antigen-binding fragment thereof, specifically binds LAR and RPTP-δ. In yet another specific embodiment, the antibody, or antigen-binding fragment thereof, specifically binds RPTP-σ and RPTP-δ. In another embodiment, the antibody or antigen-binding fragment alters immunoresponsiveness of an immune cell that expresses at least one of the RPTP polypeptides. In a specific embodiment, altering the immunoresponsiveness of the immune cell is suppressing the immunoresponsiveness of the immune cell.

In another embodiment, is provided an isolated antibody, or antigen-binding fragment thereof, that (a) specifically binds to at least one receptor-like protein tyrosine phosphatases (RPTP) polypeptide selected from (i) leukocyte common antigen-related protein (LAR); (ii) RPTP-σ; and (iii) RPTP-δ; and (b) inhibits binding of a 130L polypeptide to an immune cell that expresses at least one of (i) LAR; (ii) RPTP-σ; and (iii) RPTP-δ, wherein the amino acid sequence of the 130L polypeptide is at least 80% identical to the amino acid sequence set forth in SEQ ID NO:85 or SEQ ID NO:150. In a specific embodiment, the amino acid sequence of the 130L polypeptide (a) comprises the amino acid sequence set forth in SEQ ID NO:85 or SEQ ID NO:150; (b) is at least 95% identical to SEQ ID NO:85 or SEQ ID NO:150; (c) is at least 90% identical to SEQ ID NO:85 or SEQ ID NO:150; or (d) is at least 85% identical to SEQ ID NO:85 or SEQ ID NO:150. In certain specific embodiments, the antibody, or antigen-binding fragment thereof, specifically binds LAR and RPTP-σ. In another specific embodiment, the antibody, or antigen-binding fragment thereof, specifically binds LAR and RPTP-δ. In yet another specific embodiment, the antibody, or antigen-binding fragment thereof, specifically binds RPTP-σ and RPTP-δ. In another specific embodiment, the antibody, or antigen-binding fragment thereof, specifically binds LAR, RPTP-σ, and RPTP-δ.

Also provided herein, is an isolated antibody, or antigen-binding fragment thereof, that specifically binds to either receptor-like protein tyrosine phosphatase-sigma (RPTP-σ) or receptor-like protein tyrosine phosphatase-delta (RPTP-δ) or both, wherein binding of the antibody, or antigen-binding fragment thereof alters immunoresponsiveness of an immune cell that expresses a RPTP selected from (i) leukocyte common antigen-related protein (LAR); (ii) RPTP-σ; and (iii) RPTP-δ. In certain embodiments, altering immunoresponsiveness of the immune cell is suppressing the immunoresponsiveness of the immune cell.

In certain particular embodiments, any one of the antibodies described above and herein is a polyclonal antibody. In another particular embodiment, the antibody is a monoclonal antibody. In a certain embodiment, the monoclonal antibody is selected from a mouse monoclonal antibody, a human monoclonal antibody, a rat monoclonal antibody, and a hamster monoclonal antibody. Also provided herein is a host cell that expresses such an antibody, and in particular embodiments, the host cell is a hybridoma cell. In other embodiments, any one of the antibodies described above and herein is a humanized antibody or a chimeric antibody. Also provided herein is a host cell that expresses the humanized antibody or chimeric antibody. In other particular embodiments, the antigen-binding fragment is selected from F(ab′)₂, Fab′, Fab, Fd, and Fv. In a particular embodiment, the antigen-binding fragment is of human, mouse, chicken, or rabbit origin. In another particular embodiment, the antigen-binding fragment is a single chain Fv (scFv). An isolated antibody comprising an anti-idiotype antibody, or antigen-binding fragment thereof, that specifically binds to any one of the antibodies described above and herein. In a particular embodiment, the anti-idiotype antibody is a polyclonal antibody. In another particular embodiment, the anti-idiotype antibody is a monoclonal antibody. In another embodiment, is a composition comprising an anti-idiotype antibody, or antigen-binding fragment thereof, and a pharmaceutically suitable carrier.

Also provided herein in another embodiment, is a composition comprising any one of the antibodies, or antigen-binding fragment thereof, and a pharmaceutically suitable carrier. Also provided herein is a method of manufacture for producing any one of the antibodies, or antigen-binding fragment thereof, described above and herein.

Also provided herein is an agent that (a) specifically binds to at least one receptor-like protein tyrosine phosphatase (RPTP) polypeptide selected from (i) leukocyte common antigen-related protein (LAR); (ii) RPTP-σ; and (iii) RPTP-δ; and (b) impairs binding of a 130L polypeptide to any one of LAR, RPTP-σ, and RPTP-δ, wherein the amino acid sequence of the 130L polypeptide is at least 80% identical to the amino acid sequence set forth in either SEQ ID NO:85 or SEQ ID NO:150. In certain embodiments, the amino acid sequence of the 130L polypeptide (a) comprises the amino acid sequence set forth in SEQ ID NO:85 or SEQ ID NO:150; (b) is at least 95% identical to SEQ ID NO:85 or SEQ ID NO:150; (c) is at least 90% identical to SEQ ID NO:85 or SEQ ID NO:150; or (d) is at least 85% identical to SEQ ID NO:85 or SEQ ID NO:150. In a specific embodiment, the agent specifically binds to at least two RPTP polypeptides selected from (i) LAR; (ii) RPTP-σ; and (iii) RPTP-δ. In another specific embodiment, the agent impairs binding of the 130L polypeptide to an immune cell that expresses any one of LAR, RPTP-σ, and RPTP-δ. In a particular embodiment, the agent is selected from an antibody or antigen binding fragment thereof, a small molecule; an aptamer; and a peptide-IgFc fusion polypeptide.

Also provided herein is a composition comprising any one of the agents described above and herein and a pharmaceutically suitable carrier.

In another embodiment, a method is provided for identifying an agent that suppresses immunoresponsiveness of an immune cell comprising: (a) contacting (1) a candidate agent; (2) an immune cell that expresses at least one receptor-like protein tyrosine phosphatase (RPTP) polypeptide selected from (i) leukocyte common antigen-related protein (LAR); (ii) RPTP-σ; and (iii) RPTP-δ; and (3) a 130L polypeptide, wherein the amino acid sequence of the 130L polypeptide is at least 80% identical to the amino acid sequence set forth in SEQ ID NO:85 or SEQ ID NO:150, under conditions and for a time sufficient to permit interaction between the at least one RPTP polypeptide and the 130L polypeptide; and (b) determining a level of binding of the 130L polypeptide to the immune cell in the presence of the candidate agent and comparing a level of binding of the 130L polypeptide to the immune cell in the absence of the candidate agent, wherein a decrease in the level of binding of the 130L polypeptide to the immune cell in the presence of the candidate agent indicates that the candidate agent suppresses immunoresponsiveness of the immune cell. In certain embodiments, the amino acid sequence of the 130L polypeptide (a) comprises the amino acid sequence set forth in SEQ ID NO:85 or SEQ ID NO:150; (b) is at least 95% identical to SEQ ID NO:85 or SEQ ID NO:150; (c) is at least 90% identical to SEQ ID NO:85 or SEQ ID NO:150; or (d) is at least 85% identical to SEQ ID NO:85 or SEQ ID NO:150. In a particular embodiment, the immune cell expresses at least two RPTP polypeptides selected from (i) LAR; (ii) RPTP-σ; and (iii) RPTP-δ.

Also provided herein, in another embodiment, is a method for identifying an agent that inhibits binding of a 130L polypeptide to at least one receptor-like protein tyrosine phosphatase (RPTP) polypeptides comprising: (a) contacting (1) a candidate agent; (2) a biological sample comprising a RPTP polypeptide selected from (i) leukocyte common antigen-related protein (LAR); (ii) RPTP-σ; and (iii) RPTP-δ; and (3) the 130L polypeptide, wherein the amino acid sequence of the 130L polypeptide is at least 80% identical to the amino acid sequence set forth in SEQ ID NO:85 or SEQ ID NO:150, under conditions and for a time sufficient to permit interaction between the RPTP polypeptide and the 130L polypeptide; and (b) determining a level of binding of the 130L polypeptide to the RPTP polypeptide in the presence of the candidate agent and comparing a level of binding of the 130L polypeptide to the RPTP polypeptide in the absence of the candidate agent, wherein a decrease in the level of binding of the 130L polypeptide to the RPTP polypeptide in the presence of the candidate agent indicates that the candidate agent inhibits binding of the 130L polypeptide to the RPTP polypeptide. In certain embodiments, the amino acid sequence of the 130L polypeptide (a) comprises the amino acid sequence set forth in SEQ ID NO:85 or SEQ ID NO:150; (b) is at least 95% identical to SEQ ID NO:85 or SEQ ID NO:150; (c) is at least 90% identical to SEQ ID NO:85 or SEQ ID NO:150; or (d) is at least 85% identical to SEQ ID NO:85 or SEQ ID NO:150.

Also provided herein is a method of manufacture for producing an agent that suppresses immunoresponsiveness of an immune cell, comprising: (a) identifying an agent that suppresses immunoresponsiveness of an immune cell, wherein the step of identifying comprises: (1) contacting (i) a candidate agent; (ii) an immune cell that expresses at least one receptor-like protein tyrosine phosphatase (RPTP) polypeptide selected from leukocyte common antigen-related protein (LAR); RPTP-σ; and RPTP-δ; and (iii) a 130L polypeptide, wherein the amino acid sequence of the 130L polypeptide is at least 80% identical to the amino acid sequence set forth in SEQ ID NO:85 or SEQ ID NO:150, under conditions and for a time sufficient to permit interaction between the at least one RPTP polypeptide and the 130L polypeptide; and (2) determining a level of binding of the 130L polypeptide to the immune cell in the presence of the candidate agent and comparing a level of binding of the 130L polypeptide to the immune cell in the absence of the candidate agent, wherein a decrease in the level binding of the 130L polypeptide to the immune cell in the presence of the candidate agent indicates that the candidate agent suppresses immunoresponsiveness of the immune cell; and (b) producing the agent identified in step (a). In certain embodiments, the amino acid sequence of the 130L polypeptide (a) comprises the amino acid sequence set forth in SEQ ID NO:85 or SEQ ID NO:150; (b) is at least 95% identical to SEQ ID NO:85 or SEQ ID NO:150; (c) is at least 90% identical to SEQ ID NO:85 or SEQ ID NO:150; or (d) is at least 85% identical to SEQ ID NO:85 or SEQ ID NO:150. In a specific embodiment, the agent is selected from an antibody, or antigen-binding fragment thereof, a small molecule; an aptamer; an antisense polynucleotide; a small interfering RNA (siRNA); and a peptide-IgFc fusion polypeptide. In yet another specific embodiment, the agent is an antibody, or antigen-binding fragment thereof.

In another embodiment, a fusion polypeptide comprising a 130L polypeptide fused to an Fc polypeptide is provided. In a particular embodiment, the Fc polypeptide is a human IgG1 Fc polypeptide. In a specific embodiment, the human IgG1 Fc polypeptide is a mutein Fc polypeptide, wherein the mutein Fc polypeptide comprises the amino acid sequence of the Fc portion of a human IgG1 immunoglobulin, wherein the mutein Fc polypeptide differs from the Fc portion of a wildtype human IgG1 immunoglobulin by comprising at least two mutations, wherein a first mutation in the mutein Fc polypeptide comprises substitution of at least one amino acid in the CH2 domain such that the capability of the fusion polypeptide to bind to an IgG Fc receptor is reduced, and wherein a second mutation in the mutein Fc polypeptide is a substitution or a deletion of a cysteine residue in the hinge region, wherein the cysteine residue is the cysteine residue most proximal to the amino terminus of the hinge region of a wildtype human IgG1 immunoglobulin. In another specific embodiment, the mutein Fc polypeptide comprises substitution of at least two amino acids in the CH2 domain. In yet another specific embodiment, the mutein Fc polypeptide comprises substitution of at least three amino acids in the CH2 domain. In certain embodiments, the amino acid that is substituted is located at a position that corresponds to EU position number 235 in the CH2 domain of a human IgG immunoglobulin. In other certain embodiments, a first amino acid that is substituted is located at a position that corresponds to EU position number 234 in the CH2 domain of a human IgG immunoglobulin and a second amino acid that is substituted is located at a position that corresponds to EU position number 235 in the CH2 domain of a human IgG immunoglobulin. In another certain embodiment, a first amino acid that is substituted is located at a position that corresponds to EU position number 234 in the CH2 domain of a human IgG immunoglobulin, a second amino acid that is substituted is located at a position that corresponds to EU position number 235 in the CH2 domain of a human IgG immunoglobulin, and a third amino acid that is substituted is located at a position that corresponds to EU position number 237 in the CH2 domain of a human IgG immunoglobulin. In a particular embodiment, the leucine reside located at a position that corresponds to EU position number 235 in the CH2 domain of a human IgG immunoglobulin is substituted with a glutamic acid residue or an alanine residue. In another particular embodiment, the leucine residue located at a position that corresponds to EU position number 234 in the CH2 domain of a human IgG immunoglobulin is substituted with an alanine residue. In yet another particular embodiment, the glycine residue located at a position that corresponds to EU position number 237 in the CH2 domain of a human IgG immunoglobulin is substituted with an alanine residue. In yet another specific embodiment, the mutein Fc polypeptide further comprises substitution or deletion of at least one non-cysteine residue in the hinge region. In one particular embodiment, the mutein Fc polypeptide comprises a deletion of at least two amino acid residues in the hinge region, wherein the at least two amino acid residues include a cysteine residue and the adjacent C-terminal residue, wherein the cysteine residue is the cysteine residue most proximal to the amino terminus of the hinge region of a wildtype human IgG1 immunoglobulin. In a specific embodiment, the fusion polypeptide comprises the amino acid sequence set forth in SEQ ID NO:149.

In another embodiment, a method of suppressing an immune response in a subject is provided wherein the method comprises administering a composition that comprises a pharmaceutically suitable carrier and the fusion polypeptide comprising a 130L polypeptide fused to an Fc polypeptide as described above and herein. In a particular embodiment, the fusion polypeptide either (a) alters a biological activity of at least one of a receptor-like protein tyrosine phosphatase (RPTP) selected from (i) leukocyte common antigen-related protein (LAR); (ii) RPTP-σ; and (iii) RPTP-δ; or (b) alters a biological activity of at least two RPTP polypeptides selected from (i) LAR; (ii) RPTP-σ; and (iii) RPTP-δ. In another embodiment, a method is provided for treating an immunological disease or disorder in a subject comprising administering to the subject a pharmaceutically suitable carrier and a fusion polypeptide comprising a 130L polypeptide fused to an Fc polypeptide as described above and herein. In a specific embodiment, the fusion polypeptide either (a) alters a biological activity of at least one of receptor-like protein tyrosine phosphatase (RPTP)-σ and RPTP-δ; or (b) alters a biological activity of at least two RPTP polypeptides selected from (i) leukocyte common antigen-related protein (LAR); (ii) RPTP-σ; and (iii) RPTP-δ. In certain embodiments, the immunological disease or disorder is an autoimmune disease or an inflammatory disease. In particular embodiments, the autoimmune or inflammatory disease is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis.

In another embodiment, a method is provided for treating a disease or disorder associated with alteration of at least one of cell migration, cell proliferation, and cell differentiation in a subject comprising administering to the subject a pharmaceutically suitable carrier and a fusion polypeptide comprising a 130L polypeptide fused to an Fc polypeptide as described above and herein. In a specific embodiment, the fusion polypeptide either (a) alters a biological activity of at least one of receptor-like protein tyrosine phosphatase (RPTP)-σ or RPTP-δ; or (b) alters a biological activity of at least two RPTP polypeptides selected from (i) leukocyte common antigen-related protein (LAR); (ii) RPTP-σ; and (iii) RPTP-δ. In another specific embodiment, the disease or disorder is an immunological disease or disorder, a cardiovascular disease or disorder, a metabolic disease or disorder, or a proliferative disease or disorder. In yet another specific embodiment, the immunological disease or disorder is an autoimmune disease or an inflammatory disease. In certain embodiments, the immunological disease or disorder is multiple sclerosis, rheumatoid arthritis, systemic lupus erythematosus, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In other certain embodiments, the cardiovascular disease or disorder is atherosclerosis, endocarditis, hypertension, or peripheral ischemic disease. Also provided herein is a method of manufacture for producing the fusion polypeptide comprising a 130L polypeptide fused to an Fc polypeptide as described above and herein.

All U.S. patents, U.S. patent application publications, U.S. patent applications, foreign patents, foreign patent applications, and non-patent publications referred to in this specification and/or listed in the Application Data Sheet, are incorporated herein by reference, in their entireties.

### BRIEF DESCRIPTION OF THE DRAWINGS

FIGS. 1A-1F provide an alignment of the amino acid sequence of RPTP-σ (SEQ ID NO:29), RPTP-δ (SEQ ID NO:37), and LAR (SEQ ID NO:25). The leader peptide sequence, the immunoglobulin-like domains (1^{st} Ig domain; 2^{nd} Ig domain, 3^{rd} Ig domain); fibronectin III repeat region (FNIII); transmembrane region (TM region); and phosphatase domains (D1 and D2) of each RPTP are marked in the alignment. The first amino acid of each region is shown in bold typeface. A protease cleavage site in each phosphatase is denoted by underlining. Amino acids in regions of identity are denoted by “*” and amino acids in regions of similarity are indicated by dots. The alignment was generated using the CLUSTALW program (Thompson et al., Nucleic Acids Res. 22:4673-80 (1991)) and “GeneDoc” (Nicholas et al., EMBNEW News 4:14 (1991)).

FIG. 2 presents a schematic of an A41L fusion polypeptide encoded by a recombinant expression construct (A41LCRFC) for expression of the fusion polypeptide used for tandem affinity purification (TAP). The encoded fusion polypeptide includes mature A41L from Cowpox virus that was fused at its amino terminal end to the carboxy terminus of the human growth hormone leader peptide (GH Leader). The tandem affinity tag (CRFC) was fused to the carboxy terminus of A41L and included a human influenza virus hemagglutinin (HA) epitope (YPYDVDYA, SEQ ID NO:67) in frame with a Protein C-TAG (EDQVDPRLIDGK (SEQ ID NO:68), derived from the heavy chain of human Protein C); human rhinovirus HRV3C protease site (HRV3C cleavage site) (LEVLFQGP (SEQ ID NO:69); and a mutein derivative of the Fc portion of a human IgG immunoglobulin (Mutein FC).

FIG. 3 presents a schematic of the TAP procedure for identifying cellular polypeptides that bind to A41L.

FIG. 4 illustrates peptides of LAR, RPTP-δ, and RPTP-σ identified by tandem affinity purification (TAP) with A41L. FIG. 4A illustrates the sequences of peptides (bold typeface) within LAR (SEQ ID NO:70) that were identified by LC/MS/MS after TAP. FIG. 4B illustrates the sequences of peptides (bold typeface) within RPTP-σ (SEQ ID NO:71) that were identified by LC/MS/MS after TAP. FIG. 4C illustrates the sequences of peptides (bold typeface) within RPTP-δ (SEQ ID NO:72) that were identified by LC/MS/MS after TAP.

FIG. 5 presents an amino acid sequence alignment between (i) an A41L/Fc fusion polypeptide comprising an A41L signal peptide sequence, an A41L polypeptide, and a human IgG1 Fc polypeptide (A41L/Fc) (SEQ ID NO:74) and (ii) an A41L/mutein Fc fusion polypeptide comprising a human growth hormone signal peptide sequence, an A41L polypeptide variant, and a mutein Fc polypeptide (A41L/mutein Fc) (SEQ ID NO:73). The consensus sequence (SEQ ID NO: 75) is also shown. The vertical dotted lines indicate the amino terminal and carboxy terminal ends of the A41L polypeptide.

FIG. 6 provides an alignment of the amino acid sequence of a 130L polypeptide (GenBank Accession No. CAC21368.1) (SEQ ID NO:85) from Yaba-like Disease Virus (YLDV) and A41L (SEQ ID NO:87) (GenBank Accession No. AAM13618) from Cowpox virus.

FIG. 7 illustrates peptides of LAR, RPTP-δ, and RPTP-σ identified by tandem affinity purification (TAP) with Yaba-like Disease Virus 130L. FIG. 7A illustrates the sequences of peptides (bold typeface and underlined) within LAR (SEQ ID NO:155) that were identified by LC/MS/MS after TAP. FIG. 7B illustrates the sequences of peptides (bold typeface and underlined) within RPTP-σ (SEQ ID NO:156) that were identified by LC/MS/MS after TAP. FIG. 7C illustrates the sequences of peptides (bold typeface and underlined) within RPTP-δ (SEQ ID NO:157) that were identified by LC/MS/MS after TAP.

FIG. 8A illustrates interferon-gamma (IFN-γ) production in non-adherent peripheral blood mononuclear cells (PBMCs) in the presence of leukocyte common-antigen-related protein-human Fc conjugate (Lar-hFc). FIGS. 8B and 8C present the level of IFN-γ production in a mixed lymphocyte reaction (MLR) in the presence of Lar-hFc. Monocyte derived dendritic cells (10⁴) from donor Do476 (FIG. 8B) and from a second donor Do495 (FIG. 8C) were combined with non-adherent PBMCs to which Lar-hFc at various concentrations was added. Production of IFN-γ was determined by ELISA. Human IgG was added at the concentrations shown as a control.

FIG. 9 presents the elution profile of an LAR Ig-1-Ig-2-Ig-3-Fc fusion polypeptide that was applied to a gel filtration HPLC column.

FIG. 10 presents an immunoblot of LAR-Ig domain constructs fused to human IgG Fc, which were combined with A41lL. Complexes were isolated by immunoprecipitation with protein A. The Fc portion of the LAR-Ig-Fc constructs was detected using an anti-Fc antibody (FIG. 10A), and the presence of A41L was determined by immunoblotting with an anti-A41L antibody (FIG. 10B).

### DETAILED DESCRIPTION OF THE INVENTION

The present invention relates to the discovery that three receptor-like protein tyrosine phosphatases (RPTPs), leukocyte common-antigen-related protein (LAR), receptor protein tyrosine phosphatase-delta (RPTP-δ), and receptor protein tyrosine phosphatase-sigma (RPTP-σ), exhibit an immunoregulatory function. Expression of LAR, RPTP-δ, and RPTP-σ by immune cells was discovered by identifying polypeptides expressed by immune cells that interacted with the poxvirus polypeptides, A41L and 130L from Yaba-like Disease Virus (YLDV).

The presence of LAR on the cell surface of immune cells (e.g., a macrophages, THP-1 cell line) was shown by identifying cells that expressed polypeptides, which interacted with the poxvirus polypeptide A41L (see, e.g., U.S. Pat. No. 6,852,486). Unexpectedly, as described herein, RPTP-δ and RPTP-σ are also expressed by immune cells and bind to A41L as well as another poxvirus polypeptide 130L. Previous studies indicated that RPTP-δ and RPTP-σ are predominantly expressed in brain and nervous system tissue (see, e.g., Pulido et al., Proc. Natl. Acad. Sci. USA 92:11686-90 (1995)). More recent studies suggest that LAR, RPTP-δ, and RPTP-σ have a role in regulating axon guidance in Drosophila (see, e.g., Johnson et al., Physiol. Rev. 83:1-21 (2003)) and in development and maintenance of excitatory synapses (see, e.g., Dunah et al., Nat. Neurosci. 8:458-67 (2005)).

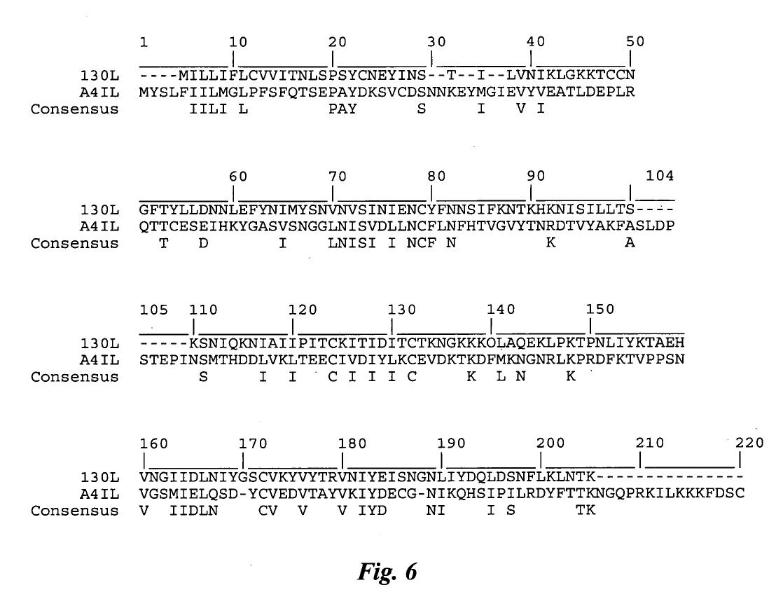

The viral polypeptide 130L that specifically binds and/or interacts with LAR, RPTP-δ, and RPTP-σ is not homologous to A41L (see FIG. 6). Yaba-like disease virus (YLDV) belongs to the Yatapoxvirus genus of the Chrodopoxyirinae. The genus has three members: tanapox virus, yaba monkey tumor virus, and YLDV. In primates YLDV causes an acute febrile illness that is characteristically accompanied by localized skin lesions (see, e.g., Knight et al., Virology 172:116-24 (1989)). The YLDV gene called 130L encodes a secreted protein having an estimated molecular weight of approximately 21 Kd (see, e.g., Lee et al., Virology 281:170-92 (2001)).

Poxvirus polypeptides, such as A41L and 130L, act at least in part in a host infected with a poxvirus to suppress an immune response specific for the virus. The suppression of an immune response in the virally infected host produces an environment in which the virus can continue replication and infection. As described herein, identifying host cells and components of the host cells, including polypeptides, that interact with poxvirus polypeptides such as A41L and 130L may lead to the development of therapeutic molecules that alter an immune response. The poxvirus polypeptides may act by inhibiting or blocking the function of host factors such as interferons, complement, cytokines, and/or chemokines, or by inhibiting, blocking, or altering, the effect of inflammation and fever (see also, e.g., U.S. Pat. No. 6,852,486). For example, in the presence of an LAR-derived polypeptide (i.e., immunoglobulin-like domains 1, 2, and 3 of LAR fused to a human IgG Fc polypeptide), peripheral blood monocytes are stimulated to produce interferon-gamma (IFN-γ). Without wishing to be bound by theory, because IFN-γ is involved in the elimination of pathogens by stimulating and inducing several aspects of the immune response, A41L may inhibit the capability of LAR to contribute to the manifestation of an immune response to the invading poxvirus by inhibiting the capability of LAR to stimulate the production of IFN-γ. Increased IFN-γ production is also associated with immunological diseases and autoimmune diseases, such as systemic lupus erythematosus (SLE). Thus, A41L, 130L, or an agent, macromolecule, or compound that mimics the interaction between A41L or 130L and LAR, for example, may be effective immunosuppressive agents. The poxvirus polypeptides, such as A41L and 130L, or other agents, polypeptides, molecules, or compounds that act like the poxvirus polypeptide to suppress immunoresponsiveness of an immune cell may be used to treat or prevent an immunological disease or disorder.

Provided herein are compositions and methods for treating diseases and disorders, including inflammatory diseases and autoimmune diseases, by contacting an immune cell with a molecule, compound, or composition that interacts with one or more of LAR, RPTP-δ, and RPTP-σ to inhibit (decrease, abrogate, suppress, prevent) immunoresponsiveness of the immune cell. Such compounds or compositions may also be useful for treating a cardiovascular disease or a metabolic disease as described herein. Alternatively, a molecule, compound, or composition that interacts with one or more of LAR, RPTP-δ, and RPTP-σ and that is useful for treatment an inflammatory or autoimmune disease, a cardiovascular, or a metabolic disease may enhance immunoresponsiveness of the immune system.

Compositions and methods are provided herein for treating or preventing, inhibiting, slowing the progression of, or reducing the symptoms associated with, an immunological disease or disorder, a cardiovascular disease or disorder, a metabolic disease or disorder, or a proliferative disease or disorder. An immunological disorder includes an inflammatory disease or disorder and an autoimmune disease or disorder. While inflammation or an inflammatory response is a host's normal and protective response to an injury, inflammation can cause undesired damage. For example, atherosclerosis is, at least in part, a pathological response to arterial injury and the consequent inflammatory cascade. Examples of immunological disorders that may be treated with an antibody or antigen-binding fragment thereof (or other agent) that binds to or interacts with one or more of LAR, RPTP-δ, and RPTP-σ described herein include but are not limited to multiple sclerosis, rheumatoid arthritis, systemic lupus erythematosus (SLE), graft versus host disease (GVHD), sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis and other inflammatory and muscle degenerative diseases (e.g., dermatomyositis, polymyositis, juvenile dermatomyositis, inclusion body myositis). A cardiovascular disease or disorder that may be treated, which may include a disease and disorder that may also be considered an immunological disease/disorder, includes for example, atherosclerosis, endocarditis, hypertension, or peripheral ischemic disease. A metabolic disease or disorder that may be treated, which may also include a disease and disorder that may also be considered an immunological disease/disorder, includes for example, diabetes, obesity, and diseases associated with abnormal or altered mitochondrial function.

As used herein, the term “isolated” means that a material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such nucleic acid or polypeptide could be part of a composition, and still be isolated in that the vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells and equivalents thereof known to those skilled in the art, and so forth. The term “comprising” (and related terms such as “comprise” or “comprises” or “having” or “including”) is not intended to exclude that in other certain embodiments, for example, an embodiment of any composition of matter, composition, method, or process, or the like, described herein may “consist of” or “consist essentially of” the described features.

### A41L Polypeptides

A41L refers to a genetic locus in viruses that are members of the poxvirus family, including for example, variola, myxoma, Shope fibroma virus, camelpox, monkeypox, ecromelia, cowpox, and vaccinia virus. The A41L gene encodes a glycoprotein (herein called A41L polypeptide) that is a viral virulence factor, which is secreted by cells infected with a poxvirus (see, e.g., International Patent Application Publication WO 98/37217; Ng et al., J. Gen. Virol. 82:2095-105 (2001)). Poxviruses, the genomes of which are double-stranded DNA, have adapted to replicate in various host species by acquiring host genes that permit the viruses to evade the host's immune system and/or to facilitate viral replication (see, e.g., Bugert et al. Virus Genes 21:111-33 (2000); Alcami et al., Immunol Today 21:447-55 (2000); McFadden et al., J. Leukoc. Biol. 57:731-38 (1995)). Polypeptides encoded by the genomes of various poxviruses may affect an immune response by inhibiting or blocking the function of host factors such interferons, complement, cytokines, and/or chemokines, or by inhibiting, blocking, or altering, the effect of inflammation and fever. For example, a recombinantly expressed A41L polypeptide binds to IFN-γ-induced chemokines, such as Mig and IP-10 (see, e.g., International Patent Application Publication WO 98/37217), and A41L binds to LAR (see, e.g., U.S. Pat. No. 6,852,486).

An A41L polypeptide as used herein refers to any one of a number of A41L polypeptides (which may be referred to in the art by nomenclature other than A41L) encoded by the genome of any one of a number of poxviruses, including but not limited to variola, myxoma, Shope fibroma virus (rabbit fibroma virus), camelpox, monkeypox, ecromelia, cowpox, and vaccinia virus (see examples of genome sequences (which include nucleotide sequences encoding A41L polypeptides) at GenBank Accession Nos. NC_{—}001559; NC_{—}001611; Y16780; X69198; NC_{—}003310; NC_{—}005337; AY603355; NC_{—}003391; AF438165; U94848; AY243312; AF380138; L22579; M35027; NC_{—}003663; X94355; AF482758; NC_{—}001132; AF170726; NC_{—}001266; AF170722; F36852 (polypeptide only). An A41L polypeptide may comprise any one of the amino acid sequences disclosed herein or known in the art, or a variant of such an amino acid sequence (including orthologues). Exemplary amino acid sequences of A41L polypeptides are set forth in SEQ ID NOs: 1-8 and at GenBank Accession Nos. NP_{—}063835 (SEQ ID NO:10); NP_{—}042191 (SEQ ID NO:11); CAA49088 (SEQ ID NO:12); NP_{—}536578 (SEQ ID NO:13); P33854 (SEQ ID NO:14); P24766 (SEQ ID NO:15); P21064 (SEQ ID NO:16); AA50551 (SEQ ID NO:17); NP_{—}570550 (SEQ ID NO:18); NP-570548 (SEQ ID NO:19); AAL73867 (SEQ ID NO:20); AAL73865 (SEQ ID NO:21).

An A41L polypeptide may also include an A41L polypeptide variant that comprises an amino acid sequence that differs by at least one amino acid from an A41L polypeptide sequence described herein or known in the art. The A41L polypeptide variant may differ from a wildtype amino acid sequence due to the insertion, deletion, addition, and/or substitution of at least one amino acid and may differ due to the insertion, deletion, addition, and/or substitution of at least two, three, four, five, six, seven, eight, nine, or ten amino acids or may differ by any number of amino acids between 10 and 45 amino acids. A41L polypeptide variants include, for example, naturally occurring polymorphisms (i.e., orthologues A41L polypeptides encoded by the genomes of different poxvirus strains) or recombinantly manipulated or engineered A41L polypeptide variants.

In certain embodiments, a variant of an A41L polypeptide retains at least one functional or biological activity of the wildtype A41L polypeptide and in other certain embodiments, an A41L polypeptide variant retains at least one, and in certain embodiments, all functions or biological activities of the wildtype A41L polypeptide. A functional or biological activity of an A41L polypeptide or a variant thereof may be determined according to methods described herein and known in the art, which function or activity includes the capability (1) to bind to or interact with at least one of, or at least two of, or all three of the receptor PTPs, LAR, RPTP-δ, and RPTP-σ; (2) to bind to an antibody that specifically binds to a wildtype A41L polypeptide; and (3) to suppress an immune response of a cell expressing at least one of LAR, RPTP-δ, and RPTP-σ. An A41L polypeptide variant that retains a functional or biological activity of a wildtype A41L polypeptide exhibits a comparable level of function or activity (that is, does not differ in a statistically significant manner) to the level of the functional or biological activity exhibited by the wildtype A41L polypeptide.

A41L polypeptide variants and polynucleotides encoding these variants can be identified by sequence comparison. As used herein, two amino acid sequences have 100% amino acid sequence identity if the amino acid residues of the two amino acid sequences are the same when aligned for maximal correspondence. Similarly, two polynucleotides have 100% nucleotide sequence identity if the nucleotide residues of the two sequences are the same when aligned for maximal correspondence. Sequence comparisons can be performed using any method including using computer algorithms well known to persons having ordinary skill in the art. Such algorithms include Align or the BLAST algorithm (see, e.g., Altschul, J. Mol. Biol. 219:555-565, 1991; Henikoff and Henikoff, Proc. Natl. Acad. Sci. USA 89:10915-10919, 1992), which are available at the NCBI website (see [online] Internet:<URL: http://www/ncbi.nlm.nih.gov/cgi-bin/BLAST). Default parameters may be used. In addition, standard software programs are available, such as those included in the LASERGENE bioinformatics computing suite (DNASTAR, Inc., Madison, Wis.); CLUSTALW program (Thompson et al., Nucleic Acids Res. 22:4673-80 (1991)); and “GeneDoc” (Nicholas et al., EMBNEW News 4:14 (1991)). Other methods for comparing two nucleotide or amino acid sequences by determining optimal alignment are practiced by those having skill in the art (see, for example, Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology, pages 123-151 (CRC Press, Inc. 1997); and Bishop (ed.), Guide to Human Genome Computing, 2nd Ed. (Academic Press, Inc. 1998)).

In certain embodiments, the amino acid sequence of an A41L polypeptide variant is at least 60%, 65%, 70%, 75%, 80%, 85%, 90%, 95%, or 98% identical to the corresponding A41L wildtype polypeptide or to an A41L polypeptide described herein and/or known in the art (see, e.g., SEQ ID NOs: 1-21). Alternatively, an A41L polypeptide variant can be identified by comparing the nucleotide sequence of a polynucleotide encoding the variant with a polynucleotide encoding an A41L polypeptide. In particular embodiments, the nucleotide sequence of a A41L polypeptide variant-encoding polynucleotide is at least 70%, 75%, 80%, 85%, 90%, or 95% identical to one or more of the polynucleotide sequences that encode A41L polypeptides, which are described herein. Polynucleotide variants also include polynucleotides that differ in nucleotide sequence identity due to the degeneracy of the genetic code but encode an A41L polypeptide having an amino acid sequence disclosed herein or known in the art.

As described herein, an A41L polypeptide, which includes A41L polypeptide variants and fragments and fusion polypeptides as described herein (which interact with or binds to at least one, two, or three of LAR, RPTP-δ, and RPTP-σ, or which interacts with or binds to at least one, two, or three of LAR, RPTP-δ, and RPTP-σ), present on the surface of a cell, may be used to alter (e.g., suppress or enhance) immunoresponsiveness of an immune cell.

In one embodiment, A41L or a variant thereof or an A41L fusion polypeptide as described herein may be used for treating a patient who presents an acute immune response. For example, an A41L polypeptide, variant, or fragment thereof may suppress an immune response associated with a disease or condition such as acute respiratory distress syndrome (ARDS). ARDS, which may develop in adults and in children, often follows a direct pulmonary or systemic insult (for example, sepsis, pneumonia, aspiration) that injures the alveolar-capillary unit. Several cytokines are associated with development of the syndrome, including, for example, tumor necrosis factor-alpha (TNF-α), interleukin-beta (IL-β), IL-10, and soluble intercellular adhesion molecule 1 (sICAM-1). The increased or decreased level of these factors and cytokines in a biological sample may be readily determined by methods and assays described herein and practiced routinely in the art to monitor the acute state and to monitor the effect of treatment.

To reduce or minimize the possibility or the extent of an immune response that is specific for A41L, the A41L, A41L variant, derivative, or fragment thereof, may be administered in a limited number of doses, may be produced or derived in a manner that alters glycosylation of A41L, may be administered under conditions that reduce or minimize antigenicity of A41L. For example, A41L may be administered prior to, concurrently with, or subsequent to the administration in the host of a second composition that suppresses an immune response, particularly a response that is specific for A41L. In addition, persons skilled in the art are familiar with methods for increasing the half-life and/or improving the pharmacokinetic properties of a polypeptide, such as by pegylating the polypeptide.

In certain other embodiments, an A41L polypeptide fragment may alter immunoresponsiveness of an immune cell. Such an A41L fragment interacts with or binds to at least one of, at least two of, or all three of the receptor PTPs, LAR, RPTP-δ, and RPTP-σ. The fragment may comprise at least 6, 7, 8, 9, 10, 11, 12, 13, 14, or 15 consecutive amino acids. In certain embodiments, the A41L fragment comprises at least any number of amino acids between 20 and 50 consecutive amino acids of an A41L polypeptide, and in other embodiments, the A41L fragment comprises at least any number of amino acids between 50 and 100 consecutive amino acids of an A41L polypeptide. A41L fragments also include truncations of an A41L polypeptide. A truncated A41L polypeptide may lack at least 1, 2-10, 11-20, 21-30, 31-40, or 50 amino acids from either the amino terminal end or the carboxy end or from both the amino terminal and carboxy end of a full-length A41L polypeptide. In certain embodiments, the A41L fragment lacks the entire amino terminal half or carboxy terminal half of the full-length A41L polypeptide. In other embodiments, the A41L polypeptide fragment (including a truncated fragment) may be conjugated, fused to, or otherwise linked to a moiety that is not an A41L polypeptide or fragment. For example, the A41L polypeptide fragment may be linked to another molecule capable of altering the immunoresponsiveness of an immune cell (e.g., suppressing the immunoresponsiveness of the immune cell), which immune cell may be the same cell, same type of cell, or a different cell than the cell affected by the A41L polypeptide or fragment.

An example of an A41L-fusion polypeptide includes an A41L polypeptide, variant, or fragment thereof as described herein fused in frame with an immunoglobulin (Ig) Fc polypeptide. An Fc polypeptide of an immunoglobulin comprises the heavy chain CH2 domain and CH3 domain and a portion of or the entire hinge region that is located between CH1 and CH2. Historically, an Fc fragment was derived by papain digestion of an immunoglobulin and included the hinge region of the immunoglobulin. Fc regions are monomeric polypeptides that may be linked into dimeric or multimeric forms by covalent (e.g., particularly disulfide bonds) and non-covalent association. The number of intermolecular disulfide bonds between monomeric subunits of Fc polypeptides varies depending on the immunoglobulin class (e.g., IgG, IgA, IgE) or subclass (e.g., human IgG1, IgG2, IgG3, IgG4, IgA1, IgA2).

Fragments of an Fc polypeptide, such as an Fc polypeptide that is truncated at the C-terminal end (that is at least 1, 2, 3, 4, 5, 10, 15, 20, or more amino acids have been removed or deleted), also may be employed. In certain embodiments, the Fc polypeptides described herein contain multiple cysteine residues, such as at least some or all of the cysteine residues in the hinge region, to permit interchain disulfide bonds to form between the Fc polypeptide portions of two separate A41L/Fc fusion proteins, thus forming A41L/Fc fusion polypeptide dimers. In other embodiments, if retention of antibody dependent cell-mediated cytotoxicity (ADCC) and complement fixation (and associated complement associated cytotoxicity (CDC)) is desired, the Fc polypeptide comprises substitutions or deletions of cysteine residues in the hinge region such that an Fc polypeptide fusion protein is monomeric and fails to form a dimer (see, e.g., U.S. Patent Application Publication No. 2005/0175614).

The Fc portion of the immunoglobulin mediates certain effector functions of an immunoglobulin. Three general categories of effector functions associated with the Fc region include (1) activation of the classical complement cascade, (2) interaction with effector cells, and (3) compartmentalization of immunoglobulins. Presently, an Fc polypeptide, and any one or more constant region domains, and fusion proteins comprising at least one immunoglobulin constant region domain can be readily prepared according to recombinant molecular biology techniques with which a skilled artisan is quite familiar.

An A41L polypeptide or variant, or fragment thereof, may be fused in frame with an immunoglobulin Fc polypeptide (A41L-Fc fusion polypeptide) that is prepared using the nucleotide and the encoded amino acid sequences derived from the animal species for whose use the A41L-IgFc fusion polypeptide is intended. A person skilled in the molecular biology art can readily prepare such fusion polypeptides according to methods described herein and practiced routinely in the art. In one embodiment, the Fc polypeptide is of human origin and may be from any of the immunoglobulin classes, such as human IgG1, IgG2, IgG3, IgG4, or IgA. In a certain embodiment, the Fc polypeptide is derived from a human IgG1 immunoglobulin (see Kabat et al., supra). In another embodiment, the A41L-Fc fusion polypeptide comprises an Fc polypeptide from a non-human animal, for example, but not limited to, a mouse, rat, rabbit, or hamster. The amino acid sequence of an Fc polypeptide derived from an immunoglobulin of a host species to which an A41L-Fc fusion polypeptide may be administered is likely to be less immunogenic or non-immunogenic than an Fc polypeptide from a non-syngeneic host. As described herein, immunoglobulin sequences of a variety of species are available in the art, for example, in Kabat et al. (in Sequences of Proteins of Immunological Interest, 4th ed., (U.S. Dept. of Health and Human Services, U.S. Government Printing Office, 1991)).

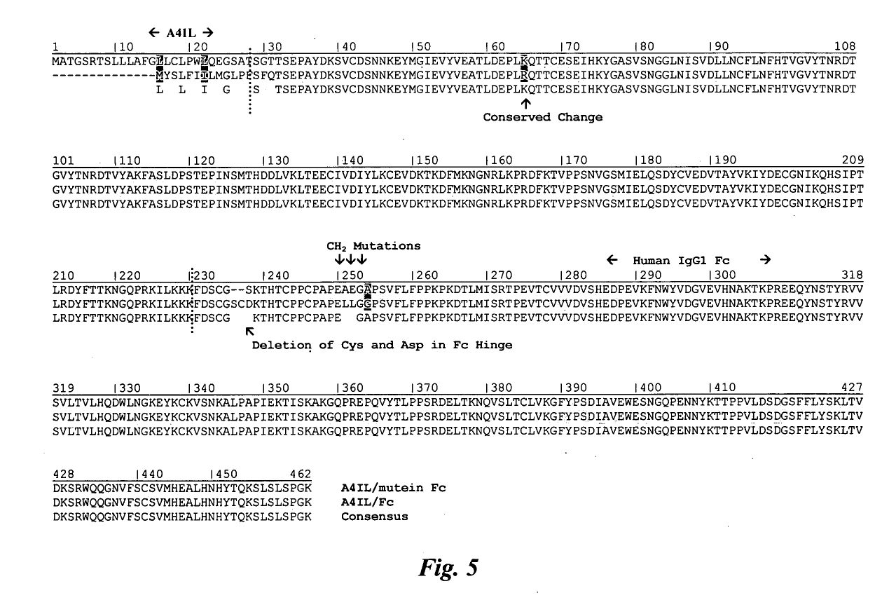

As described herein an A41L polypeptide (or variant or fragment thereof) that is fused in frame to an Fc polypeptide may comprise any one of the A41L polypeptides disclosed herein or known in the art. For example, an A41L polypeptide having the amino acid sequence of the A41L polypeptide encoded by the genome of the cowpox Brighton Red strain may be fused in frame to an immunoglobulin Fc region. Also as described herein, the Fc portion of the fusion polypeptide may be derived from a human or non-human immunoglobulin. By way of example, the Fc portion of an A41L-Fc fusion polypeptide may comprise the amino acid sequence of all or a portion of the hinge region, CH2 domain, and CH3 domain of a human immunoglobulin, for example, an IgG1. Such an exemplary fusion polypeptide is depicted in FIG. 5. An A41L-Fc fusion polypeptide may further comprise a signal peptide sequence that facilitates post-translational transport of the polypeptide in the host cell in which the fusion polypeptide is expressed. The signal peptide sequence may be derived from an A41L signal peptide sequence encoded by the poxvirus genome from which the A41L sequence was obtained. Alternatively, the signal peptide sequence may comprise an amino acid sequence that is derived from an unrelated polypeptide, such as human growth hormone.

An Fc polypeptide as described herein also includes Fc polypeptide variants. One such Fc polypeptide variant has one or more cysteine residues (such as one or more cysteine residues in the hinge region) that forms an interchain disulfide bond substituted with another amino acid, such as serine, to reduce the number of interchain disulfide bonds that can form between the two heavy chain constant region polypeptides that form an Fc polypeptide. In addition, or alternatively, the most amino terminal cysteine residue of the hinge region that forms a disulfide bond with a light chain constant region in a complete immunoglobulin molecule may be substituted, for example, with a serine residue. Alternatively, one or more cysteine residues may be deleted from the wildtype hinge of the Fc polypeptide. Another example of an Fc polypeptide variant is a variant that has one or more amino acids involved in an effector function substituted or deleted such that the Fc polypeptide has a reduced level of an effector function. For example, amino acids in the Fc region may be substituted to reduce or abrogate binding of a component of the complement cascade (see, e.g., Duncan et al., Nature 332:563-64 (1988); Morgan et al., Immunology 86:319-24 (1995)) or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity. Such an Fc polypeptide variant that differs from the wildtype Fc polypeptide is also called herein a mutein Fc polypeptide.

In one embodiment, an A41L polypeptide (or fragment or variant thereof) is fused in frame with an Fc polypeptide that comprises at least one substitution of a residue that in the wildtype Fc region polypeptide contributes to binding of an Fc polypeptide or immunoglobulin to one or more IgG Fc receptors expressed on certain immune cells. Such a mutein Fc polypeptide comprises at least one substitution of an amino acid residue in the CH2 domain of the mutein Fc polypeptide, such that the capability of the fusion polypeptide to bind to an IgG Fc receptor, such as an IgG Fc receptor present on the surface of an immune cell, is reduced.

By way of background, on human leukocytes three distinct types of Fc IgG-receptors are expressed that are distinguishable by structural and functional properties, as well as by antigenic structures, which differences are detected by CD specific monoclonal antibodies. The IgG Fc receptors are designated FcγRI (CD64), FcγRII (CD32), and FcγRIII (CD16) and are differentially expressed on overlapping subsets of leukocytes.

FcγRI (CD64), a high-affinity receptor expressed on monocytes, macrophages, neutrophils, myeloid precursors, and dendritic cells, comprises isoforms la and lb. FcγRII (CD32), comprised of isoforms IIa, llb1, llb2, llb3, and llc, is a low-affinity receptor that is the most widely distributed human FcγR type; it is expressed on most types of blood leukocytes, as well as on Langerhans cells, dendritic cells, and platelets. FcγRlll (CD16) has two isoforms, both of which are capable of binding to human IgG1 and IgG3. The FcγRIlla isoform has an intermediate affinity for IgG and is expressed on macrophages, monocytes, natural killer (NK) cells, and subsets of T cells. FcγRlllb is a low-affinity receptor for IgG and is selectively expressed on neutrophils.

Residues in the amino terminal portion of the CH2 domain that contribute to IgG Fc receptor binding include residues at positions Leu234-Ser239 (Leu-Leu-Gly-Gly-Pro-Ser (SEQ ID NO:80) (EU numbering system, Kabat et al., supra) (see, e.g., Morgan et al., Immunology 86:319-24 (1995), and references cited therein). These positions correspond to positions 15-20 of the amino acid sequence of a human IgG1 Fc polypeptide (SEQ ID NO:79). Substitution of the amino acid at one or more of these six positions (i.e., one, two, three, four, five, or all six) in the CH2 domain results in a reduction of the capability of the Fc polypeptide to bind to one or more of the IgG Fc receptors (or isoforms thereof) (see, e.g., Burton et al., Adv. Immunol. 51:1 (1992); Hulett et al., Adv. Immunol. 57:1 (1994); Jefferis et al., Immunol. Rev. 163:59 (1998); Lund et al., J. Immunol. 147:2657 (1991); Sarmay et al., Mol. Immunol. 29:633 (1992); Lund et al., Mol. Immunol. 29:53 (1992); Morgan et al., supra). In addition to substitution of one or more amino acids at EU positions 234-239, one, two, or three or more amino acids adjacent to this region (either to the carboxy terminal side of position 239 or to the amino terminal side of position 234) may also be substituted.

By way of example, substitution of the leucine residue at position 235 (which corresponds to position 16 of SEQ ID NO:79) with a glutamic acid residue or an alanine residue abolishes or reduces, respectively, the affinity of an immunoglobulin (such as human IgG3) for FcγRI (Lund et al., 1991, supra; Canfield et al., supra; Morgan et al., supra). As another example, replacement of the leucine residues at positions 234 and 235 (which correspond to positions 15 and 16 of SEQ ID NO:79), for example, with alanine residues, abrogates binding of an immunoglobulin to FcγRIIa (see, e.g., Wines et al., supra). Alternatively, leucine at position 234 (which corresponds to position 15 of SEQ ID NO:79), leucine at position 235 (which corresponds to position 16 of SEQ ID NO:79), and glycine at position 237 (which corresponds to position 18 of SEQ ID NO:79), each may be substituted with a different amino acid, such as leucine at position 234 may be substituted with an alanine residue (L234A), leucine at 235 may be substituted with an alanine residue (L235A) or with a glutamic acid residue (L235E), and the glycine residue at position 237 may be substituted with another amino acid, for example an alanine residue (G237A).

In one embodiment, a mutein Fc polypeptide that is fused in frame to a viral polypeptide (or variant or fragment thereof) comprises one, two, three, four, five, or six mutations at positions 15-20 of SEQ ID NO:79 that correspond to positions 234-239 of a human IgG1 CH2 domain (EU numbering system) as described herein. An exemplary mutein Fc polypeptide has the amino acid sequence set forth in SEQ ID NO:77 in which substitutions corresponding to (L234A), (L235E), and (G237A) may be found at positions 13, 14, and 16 of SEQ ID NO:77.

In another embodiment, a mutein Fc polypeptide comprises a mutation of a cysteine residue in the hinge region of an Fc polypeptide. In one embodiment, the cysteine residue most proximal to the amino terminus of the hinge region of an Fc polypeptide (e.g., for example, the cysteine residue most proximal to the amino terminus of the hinge region of the Fc portion of a wildtype IgG1 immunoglobulin) is deleted or substituted with another amino acid. That is, by way of illustration, the cysteine residue at position 1 of SEQ ID NO:79 is deleted, or the cysteine residue at position 1 is substituted with another amino acid that is incapable of forming a disulfide bond, for example, with a serine residue. In another embodiment, a mutein Fc polypeptide comprises a deletion or substitution of the cysteine residue most proximal to the amino terminus of the hinge region of an Fc polypeptide further comprises deletion or substitution of the adjacent C-terminal amino acid. In a certain embodiment, this cysteine residue and the adjacent C-terminal residue are both deleted from the hinge region of a mutein Fc polypeptide. In a specific embodiment, the cysteine residue at position 1 of SEQ ID NO:79 and the aspartic acid at position 2 of SEQ ID NO:79 are deleted. Fc polypeptides that comprise deletion of these cysteine and aspartic acid residues in the hinge region may be efficiently expressed in a host cell, and in certain instances, may be more efficiently expressed in a cell than an Fc polypeptide that retains the wildtype cysteine and aspartate residues.

In a specific embodiment, a mutein Fc polypeptide comprises the amino acid sequence set forth in SEQ ID NO:77, which differs from the wildtype Fc polypeptide (SEQ ID NO:79) wherein the cysteine residue at position 1 of SEQ ID NO:79 is deleted and the aspartic acid at position 2 of SEQ ID NO:79 is deleted and the leucine reside at position 15 of SEQ ID NO:79 is substituted with an alanine residue, the leucine residue at position 16 is substituted with a glutamic acid residue, and the glycine at position 18 is substituted with an alanine residue (see also FIG. 5). Thus, an exemplary mutein Fc polypeptide comprises an amino acid sequence at its amino terminal portion of KTHTCPPCPAPEAEGAPS (SEQ ID NO:81) (see SEQ ID NO:77, an exemplary Fc mutein sequence).

Other Fc variants encompass similar amino acid sequences of known Fc polypeptide sequences that have only minor changes, for example by way of illustration and not limitation, covalent chemical modifications, insertions, deletions and/or substitutions, which may further include conservative substitutions. Amino acid sequences that are similar to one another may share substantial regions of sequence homology. Similarly, nucleotide sequences that encode the Fc variants may encompass substantially similar nucleotide sequences and have only minor changes, for example by way of illustration and not limitation, covalent chemical modifications, insertions, deletions, and/or substitutions, which may further include silent mutations owing to degeneracy of the genetic code. Nucleotide sequences that are similar to one another may share substantial regions of sequence homology.

An Fc polypeptide or at least one immunogloblulin constant region, or portion thereof, when fused to a peptide or polypeptide of interest acts, at least in part, as a vehicle or carrier moiety that prevents degradation and/or increases half-life, reduces toxicity, reduces immunogenicity, and/or increases biological activity of the peptide such as by forming dimers or other multimers (see, e.g., U.S. Pat. Nos. 6,018,026; 6,291,646; 6,323,323; 6,300,099; 5,843,725). (See also, e.g., U.S. Pat. No. 5,428,130; U.S. Pat. No. 6,660,843; U.S. Patent Application Publication Nos. 2003/064480; 2001/053539; 2004/087778; 2004/077022; 2004/071712; 2004/057953/ 2004/053845/ 2004/044188; 2004/001853; 2004/082039).

An A41L polypeptide (or variant or fragment thereof) fused in frame with an Fc polypeptide or Fc polypeptide variant (e.g., a mutein Fc polypeptide) may comprise a peptide linker between the A41L polypeptide and Fc polypeptide. The linker may be a single amino acid (such as for example a glycine residue) or may be two, three, four, five, six, seven, eight, nine, or ten amino acids, or may be any number of amino acids between 10 and 20 amino acids. By way of illustration but not limitation, a linker may comprise at least two amino acids that are encoded by a nucleotide sequence that is a restriction enzyme recognition site. Examples of such restriction enzyme recognition sites include, for example, BamHI, ClaI, EcoRI, HindIII, KpnI, NcoI, NheI, PmlI, PstI, SalI, and XhoI.

An A41L polypeptide, fragment thereof, or variant thereof, fused in frame with a mutein Fc polypeptide may be used to suppress an immune response in a subject when administered with a pharmaceutically or physiologically suitable carrier or excipient according to methods described herein and known to practitioners in the medical art. Such fusion polypeptides may alter a biological activity of at least one of the RPTP polypeptides described herein (i.e., LAR, RPTP-σ, RPTP-δ), at least two of the RPTP polypeptides or all three RPTP polypeptides. In certain embodiments, an A41L polypeptide, fragment thereof, or variant thereof, fused in frame with a mutein Fc polypeptide is used for treating an immunological disease or disorder (including an autoimmune disease or an inflammatory disease), which are described in detail herein. As described herein, the A41l/mutein Fc polypeptides may also be used to treat a disease or disorder associated with alteration of cell migration, cell proliferation, or cell differentiation, which includes but is not limited to an immunological disease or disorder, a cardiovascular disease or disorder, a metabolic disease or disorder, or a proliferative disease or disorder.

A41L polypeptide fragments include A41L polypeptide variant fragments. A41L polypeptide fragments also include A41L fragments having an amino acid sequence that differs from the full-length A41L from which the fragments were derived, that is the A41L polypeptide fragment variant has at least 99%, 98%, 97%, 95%, 90%, 87%, 85%, or 80% amino acid sequence identity with a portion of the full-length A41L polypeptide. Variants of A41L polypeptide fragments that have the capability to alter (suppress or enhance) the immunoresponsiveness of an immune cell retain comparable capability to alter the immunoresponsiveness of an immune cell.

A41L polypeptide variants and A41L polypeptide fragment variants that retain the capability to alter immunoresponsiveness of an immune cell include variants that contain conservative amino acid substitutions. A variety of criteria known to persons skilled in the art indicate whether amino acids at a particular position in a peptide or polypeptide are conservative (or similar). For example, a similar amino acid or a conservative amino acid substitution is one in which an amino acid residue is replaced with an amino acid residue having a similar side chain, such as amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, or the BLAST algorithm, as described herein). By way of example, an A41L variant described herein has a conservative substitution of an arginine residue with a lysine residue at position 50 of SEQ ID NO:82 (GenBank Acc. No. AAM13618, May 20, 2003) to provide SEQ ID NO:83 (see also, e.g., Hu et al., Virology 181:716-20 (1991); Hu et al., Virology 204:343-56 (1994)). This A41L variant retains the functions and properties of the wild type A41L polypeptide.

An A41L polypeptide variant also includes a variant that interacts with or binds to only one or two (i.e., LAR and RPTP-δ, LAR and RPTP-σ, or RPTP-δ and RPTP-σ) but not all three of LAR, RPTP-δ, and RPTP-σ. Such a variant comprises at least 1, 2, 3, 4, 5, 6, 7, 8, 9, 10, 11-15, 16-25, 26-35, or 36-45 amino acid substitutions, deletions, or insertions compared with the wildtype A41L polypeptide. Binding of A41L to each of the RPTPs may be determining according to methods described herein and practiced in the art. The source of the polypeptides for binding studies include, for example, isolated A41L and RPTPs, or fragments thereof, or individual cell lines capable of recombinant expression of one of A41L, LAR, RPTP-δ, and RPTP-σ.

Variants of A41L full-length polypeptides or A41L fragments may be readily prepared by genetic engineering and recombinant molecular biology methods and techniques. Analysis of the primary and secondary amino acid sequence of an A41L polypeptide and computer modeling to analyze the tertiary structure of the polypeptide may aid in identifying specific amino acid residues that can be substituted without altering the structure and as a consequence, potentially the function, of the A41L polypeptide. Modification of DNA encoding an A41L polypeptide or fragment may be performed by a variety of methods, including site-specific or site-directed mutagenesis of the DNA, which methods include DNA amplification using primers to introduce and amplify alterations in the DNA template, such as PCR splicing by overlap extension (SOE). Mutations may be introduced at a particular location by synthesizing oligonucleotides containing a mutant sequence, flanked by restriction sites enabling ligation to fragments of the native sequence. Following ligation, the resulting reconstructed sequence encodes a variant (or derivative) having the desired amino acid insertion, substitution, or deletion.

Site-directed mutagenesis is typically effected using a phage vector that has single- and double-stranded forms, such as an M13 phage vector, which is well-known and commercially available. Other suitable vectors that contain a single-stranded phage origin of replication may be used (see, e.g., Veira et al., Meth. Enzymol. 15:3 (1987)). In general, site-directed mutagenesis is performed by preparing a single-stranded vector that encodes the protein of interest. An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. Additional disclosure relating to site-directed mutagenesis may be found, for example, in Kunkel et al. (Meth. Enzymol. 154:367 (1987)) and in U.S. Pat. Nos. 4,518,584 and 4,737,462. The heteroduplex is introduced into appropriate bacterial cells, and clones that include the desired mutation are selected. The resulting altered DNA molecules may be expressed recombinantly in appropriate host cells to produce the variant, modified protein.

Oligonucleotide-directed site-specific (or segment specific) mutagenesis procedures may be employed to provide an altered polynucleotide that has particular codons altered according to the substitution, deletion, or insertion desired. Deletion or truncation derivatives of proteins may also be constructed by using convenient restriction endonuclease sites adjacent to the desired deletion. Subsequent to restriction, overhangs may be filled in and the DNA religated. Exemplary methods of making the alterations set forth above are disclosed by Sambrook et al. (Molecular Cloning: A Laboratory Manual, 3d ed., Cold Spring Harbor Laboratory Press, NY 2001). Alternatively, random mutagenesis techniques, such as alanine scanning mutagenesis, error prone polymerase chain reaction mutagenesis, and oligonucleotide-directed mutagenesis may be used to prepare A41L polypeptide variants and fragment variants (see, e.g., Sambrook et al., supra).

Assays for assessing whether the variant folds into a conformation comparable to the non-variant polypeptide or fragment include, for example, the ability of the protein to react with mono- or polyclonal antibodies that are specific for native or unfolded epitopes, the retention of ligand-binding functions, and the sensitivity or resistance of the mutant protein to digestion with proteases (see Sambrook et al., supra). A41L variants as described herein can be identified, characterized, and/or made according to these methods described herein or other methods known in the art, which are routinely practiced by persons skilled in the art.

Mutations that are made or identified in the nucleic acid molecules encoding an A41L polypeptide preferably preserve the reading frame of the coding sequences. Furthermore, the mutations will preferably not create complementary regions that when transcribed could hybridize to produce secondary mRNA structures, such as loops or hairpins, that would adversely affect translation of the mRNA. Although a mutation site may be predetermined, the nature of the mutation per se need not be predetermined. For example, to select for optimum characteristics of a mutation at a given site, random mutagenesis may be conducted at the target codon and the expressed mutants screened for gain or loss or retention of biological activity.

An A41L polynucleotide is any polynucleotide that encodes an A41L polypeptide or at least a portion (or fragment) of an A41L polypeptide or a variant thereof, or that is complementary to such a polynucleotide. The nucleotide sequences of polynucleotides that encode A41L, or its orthologues, may be found, for example, in the genomic sequences of poxviruses provided in GenBank entries for which Accession numbers are provided herein, in GenBank Accession Nos. NC_{—}001559; NC_{—}001611; Y16780; X69198; NC_{—}003310; NC_{—}005337; AY603355; NC_{—}003391; AF438165; U94848; AY243312; AF380138; L22579; M35027; NC_{—}003663; X94355; AF482758; NC_{—}001132; AF170726; NC_{—}001266; AF170722 and that can be deduced from the amino acid sequences disclosed herein (e.g., SEQ ID NOs:1-21). Polynucleotides comprise at least 15 consecutive nucleotides or at least 30, 35, 40, 50, 55, or 60 consecutive nucleotides, in certain embodiments at least 70, 75, 80, 90, 100, 110, 120, 125, or 130 consecutive nucleotides, and in other embodiments at least 135, 140, 145, 150, 155, 160, or 170 consecutive nucleotides, and in other embodiments at least 180, 190, 200, 225, 250, 275, 300, 325, 350, 375, 400, 405, 410, 420, 425, 445, 450, 475, 500, 525, 530, 545, 550, 575, 600, 625, 650, or 660 consecutive nucleotides that include sequences encoding an A41L polypeptide, or nucleotide sequences that are complementary to such a sequence. Certain polynucleotides that encode an A41L polypeptide, variant, or fragment thereof may also be used as probes, primers, short interfering RNA (siRNA), or antisense oligonucleotides, as described herein. Polynucleotides may be single-stranded DNA or RNA (coding or antisense) or double-stranded RNA (e.g., genomic or synthetic) or DNA (e.g., cDNA or synthetic).

Polynucleotide variants may also be identified by hybridization methods. Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5×, and 0.2×SSC containing 0.05-0.1% SDS. For additional stringency, conditions may include a wash in 0.1×SSC and 0.1% SDS at 50-60° C. for 15 minutes. As understood by persons having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used (i.e., for example, the guanine plus cytosine (G/C) versus adenine plus thymidine (A/T) content). Accordingly, a person skilled in the art will appreciate that suitably stringent conditions can be readily selected without undue experimentation when a desired selectivity of the probe is identified.

### 130L Polypeptides

As described herein the 130L gene encodes a glycoprotein (herein called 130L polypeptide) that is likely a viral virulence factor and that is secreted by cells infected with YLDV. Similar to other poxviruses, the genome of YLDV is double-stranded DNA, and has the virus has adapted to replicate in various host species by acquiring host genes that permit the viruses to evade the host's immune system and/or to facilitate viral replication (see, e.g., Najarro et al., J. Gen. Virol. 84:3325-36 (2003)). Polypeptides encoded by the genomes of various poxviruses may affect an immune response by inhibiting or blocking the function of host factors such interferons, complement, cytokines, and/or chemokines, or by inhibiting, blocking, or altering, the effect of inflammation and fever.

A 130L polypeptide as used herein refers to any one of a number of 130L polypeptides encoded by the genome of the yatapoxvirus Yaba-like disease virus (see examples of genome sequences (which include nucleotide sequences encoding 130L polypeptides) for Yaba-like disease virus at GenBank Accession Nos. AJ293568.1 and NC_{—}002642.1). A 130L polypeptide may comprise any one of the amino acid sequences disclosed herein or known in the art, or a variant of such an amino acid sequence (including orthologues). Exemplary amino acid sequences of 130L polypeptides are set forth in SEQ ID NO:85 (see GenBank Accession No. CAC21368.1) and GenBank Accession No. NP_{—}073515.1 (SEQ ID NO:144).

A 130L polypeptide may also include a 130L polypeptide variant that comprises an amino acid sequence that differs by at least one amino acid from a 130L polypeptide sequence described herein or known in the art. The 130L polypeptide variant may differ from a wildtype amino acid sequence due to the insertion, deletion, addition, and/or substitution of at least one amino acid and may differ due to the insertion, deletion, addition, and/or substitution of at least two, three, four, five, six, seven, eight, nine, or ten amino acids or may differ by any number of amino acids between 10 and 45 amino acids. 130L polypeptide variants include, for example, a naturally occurring polymorphism (i.e., orthologues of 130L polypeptides encoded by the genomes of different yatapoxvirus strains) or recombinantly manipulated or engineered 130L polypeptide variants.

In certain embodiments, a variant of a 130L polypeptide retains at least one functional or biological activity of the wildtype 130L polypeptide and in other certain embodiments, a 130L polypeptide variant retains at least one, and in certain embodiments, all functions or biological activities of the wildtype 130L polypeptide. A functional or biological activity of 130L polypeptide or a variant thereof may be determined according to methods described herein and known in the art, which function or activity includes the capability (1) to bind to or interact with at least one of, or at least two of, or all three of the receptor PTPs, LAR, RPTP-δ, and RPTP-σ; (2) to bind to an antibody that specifically binds to a wildtype 130L polypeptide; and (3) to suppress an immune response of a cell expressing at least one of LAR, RPTP-δ, and RPTP-σ. A 130L polypeptide variant that retains a functional or biological activity of a wildtype 130L polypeptide exhibits a comparable level of function or activity (that is, does not differ in a statistically significant or biologically significant manner) to the level of the functional or biological activity exhibited by the wildtype 130L polypeptide.

130L polypeptide variants and polynucleotides encoding these variants can be identified by sequence comparison. As used herein, two amino acid sequences have 100% amino acid sequence identity if the amino acid residues of the two amino acid sequences are the same when aligned for maximal correspondence. Similarly, two polynucleotides have 100% nucleotide sequence identity if the nucleotide residues of the two sequences are the same when aligned for maximal correspondence. Sequence comparisons can be performed using any method including using computer algorithms well known to persons having ordinary skill in the art. Such algorithms include Align or the BLAST algorithm (see, e.g., Altschul, J. Mol. Biol. 219:555-565, 1991; Henikoff and Henikoff, Proc. Natl. Acad. Sci. USA 89:10915-10919, 1992), which are available at the NCBI website (see [online] Internet:<URL: http://www/ncbi.nlm.nih.gov/cgi-bin/BLAST). Default parameters may be used. In addition, standard software programs are available, such as those included in the LASERGENE bioinformatics computing suite (DNASTAR, Inc., Madison, Wis.); CLUSTALW program (Thompson et al., Nucleic Acids Res. 22:4673-80 (1991)); and “GeneDoc” (Nicholas et al., EMBNEW News 4:14 (1991)). Other methods for comparing two nucleotide or amino acid sequences by determining optimal alignment are practiced by those having skill in the art (see, for example, Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology, pages 123-151 (CRC Press, Inc. 1997); and Bishop (ed.), Guide to Human Genome Computing, 2nd Ed. (Academic Press, Inc. 1998)).

In certain embodiments, the amino acid sequence of a 130L polypeptide variant is at least 60%, 65%, 70%, 75%, 80%, 85%, 90%, 95%, or 98% identical to the corresponding 130L wildtype polypeptide or to a 130L polypeptide described herein and/or known in the art (see, e.g., SEQ ID NO:85 (which has the signal peptide sequence (SEQ ID NO:151)) or SEQ ID NO:150 (mature 130L polypeptide)). Alternatively, a 130L polypeptide variant can be identified by comparing the nucleotide sequence of a polynucleotide encoding the variant with a polynucleotide encoding a 130L polypeptide. In particular embodiments, the nucleotide sequence of a 130L polypeptide variant-encoding polynucleotide is at least 70%, 75%, 80%, 85%, 90%, or 95% identical to one or more of the polynucleotide sequences that encode 130L polypeptides, which are described herein. Polynucleotide variants also include polynucleotides that differ in nucleotide sequence identity due to the degeneracy of the genetic code but encode a 130L polypeptide having an amino acid sequence disclosed herein or known in the art.

As described herein, a 130L polypeptide, which includes 130L polypeptide variants and fragments and fusion polypeptides as described herein (which interact with or binds to at least one, two, or three of LAR, RPTP-δ, and RPTP-σ), present on the surface of a cell, may be used to alter (e.g., suppress or enhance) immunoresponsiveness of an immune cell. In one embodiment, a 130L polypeptide or a variant thereof or a 130L fusion polypeptide as described herein may be used for treating a patient who presents an acute immune response. For example, a 130L polypeptide, variant, or fragment thereof may suppress an immune response associated with a disease or condition such as acute respiratory distress syndrome (ARDS). ARDS, which may develop in adults and in children, often follows a direct pulmonary or systemic insult (for example, sepsis, pneumonia, aspiration) that injures the alveolar-capillary unit. Several cytokines are associated with development of the syndrome, including, for example, tumor necrosis factor-alpha (TNF-α), interleukin-beta (IL-β), IL-10, and soluble intercellular adhesion molecule 1 (sICAM-1). The increased or decreased level of these factors and cytokines in a biological sample may be readily determined by methods and assays described herein and practiced routinely in the art to monitor the acute state and to monitor the effect of treatment.

To reduce or minimize the possibility or the extent of an immune response that is specific for 130L, the 130L polypeptide, 130L variant, derivative, or fragment thereof, or fusion protein comprising same may be administered in a limited number of doses, may be produced or derived in a manner that alters glycosylation of 130L, and/or may be administered under conditions that reduce or minimize antigenicity of 130L. For example, 130L may be administered prior to, concurrently with, or subsequent to the administration in the host of a second composition that suppresses an immune response, particularly a response that is specific for 130L.

In certain other embodiments, a 130L polypeptide fragment may alter immunoresponsiveness of an immune cell. Such a 130L fragment interacts with or binds to at least one of, at least two of, or all three of the receptor PTPs, LAR, RPTP-δ, and RPTP-σ. The fragment may comprise at least 6, 7, 8, 9, 10, 11, 12, 13, 14, or 15 consecutive amino acids. In certain embodiments, the 130L fragment comprises at least any number of amino acids between 20 and 50 consecutive amino acids of a 130L polypeptide, and in other embodiments, the 130L fragment comprises at least any number of amino acids between 50 and 100 consecutive amino acids of a 130L polypeptide. 130L fragments also include truncations of a 130L polypeptide. A truncated 130L polypeptide may lack at least 1, 2-10, 11-20, 21-30, 31-40, or 50 amino acids from either the amino terminal end or the carboxy end or from both the amino terminal and carboxy end of a full-length 130L polypeptide. In certain embodiments, the 130L fragment lacks the entire amino terminal half or carboxy terminal half of the full-length 130L polypeptide. In other embodiments, the 130L polypeptide fragment (including a truncated fragment) may be conjugated, fused to, or otherwise linked to a moiety that is not a 130L polypeptide or fragment. For example, the 130L polypeptide fragment may be linked to another molecule capable of altering the immunoresponsiveness of an immune cell (e.g., suppressing the immunoresponsiveness of the immune cell), which immune cell may be the same cell, same type of cell, or a different cell than the cell affected by the 130L polypeptide or fragment. In addition, persons skilled in the art are familiar with methods for increasing the half-life and/or improving the pharmacokinetic properties of a polypeptide, such as by pegylating the polypeptide.

An example of a 130L-fusion polypeptide includes a 130L polypeptide, variant, or fragment thereof as described herein fused in frame with an immunoglobulin (Ig) Fc polypeptide. An Fc polypeptide of an immunoglobulin comprises the heavy chain CH2 domain and CH3 domain and a portion of or the entire hinge region that is located between CH1 and CH2. Historically, an Fc fragment was derived by papain digestion of an immunoglobulin and included the hinge region of the immunoglobulin. Fc regions are monomeric polypeptides that may be linked into dimeric or multimeric forms by covalent (e.g., particularly disulfide bonds) and non-covalent association. The number of intermolecular disulfide bonds between monomeric subunits of Fc polypeptides varies depending on the immunoglobulin class (e.g., IgG, IgA, IgE) or subclass (e.g., human IgG1, IgG2, IgG3, IgG4, IgA1, IgGA2).

Fragments of an Fc polypeptide, such as an Fc polypeptide that is truncated at the C-terminal end (that is at least 1, 2, 3, 4, 5, 10, 15, 20, or more amino acids have been removed or deleted), also may be employed. In certain embodiments, the Fc polypeptides described herein contain multiple cysteine residues, such as at least some or all of the cysteine residues in the hinge region, to permit interchain disulfide bonds to form between the Fc polypeptide portions of two separate 130L/Fc fusion proteins, thus forming 130L/Fc fusion polypeptide dimers. In other embodiments, if retention of antibody dependent cell-mediated cytotoxicity (ADCC) and complement fixation (and associated complement associated cytotoxicity (CDC)) is desired, the Fc polypeptide comprises substitutions or deletions of cysteine residues in the hinge region such that an Fc polypeptide fusion protein is monomeric and fails to form a dimer (see, e.g., U.S. Patent Application Publication No. 2005/0175614).

The Fc portion of the immunoglobulin mediates certain effector functions of an immunoglobulin. Three general categories of effector functions associated with the Fc region include (1) activation of the classical complement cascade, (2) interaction with effector cells, and (3) compartmentalization of immunoglobulins. Presently, an Fc polypeptide, and any one or more constant region domains, and fusion proteins comprising at least one immunoglobulin constant region domain can be readily prepared according to recombinant molecular biology techniques with which a skilled artisan is quite familiar.

A 130L polypeptide or variant, or fragment thereof, may be fused in frame with an immunoglobulin Fc polypeptide (130L-Fc fusion polypeptide) that is prepared using the nucleotide and the encoded amino acid sequences derived from the animal species for whose use the 130L-IgFc fusion polypeptide is intended. A person skilled in the molecular biology art can readily prepare such fusion polypeptides according to methods described herein and practiced routinely in the art. In one embodiment, the Fc polypeptide is of human origin and may be from any of the immunoglobulin classes and subclasses, such as human IgG1, IgG2, IgG3, IgG4, or IgA. In a certain embodiment, the Fc polypeptide is derived from a human IgG1 immunoglobulin (see Kabat et al., supra). In another embodiment, the 130L-Fc fusion polypeptide comprises an Fc polypeptide from a non-human animal, for example, but not limited to, a mouse, rat, rabbit, or hamster. The amino acid sequence of an Fc polypeptide derived from an immunoglobulin of a host species to which a 130L-Fc fusion polypeptide may be administered is likely to be less immunogenic or non-immunogenic than an Fc polypeptide from a non-syngeneic host. As described herein, immunoglobulin sequences of a variety of species are available in the art, for example, in Kabat et al. (in Sequences of Proteins of Immunological Interest, 4th ed., (U.S. Dept. of Health and Human Services, U.S. Government Printing Office, 1991)).

As described herein a 130L polypeptide (or variant or fragment thereof) that is fused to an Fc polypeptide may comprise any one of the 130L polypeptides disclosed herein or known in the art. For example, a 130L polypeptide having the amino acid sequence of the 130L polypeptide encoded by the genome of a Yaba-like disease virus (see, e.g., GenBank Accession Nos. AJ293568.1 and NC_{—}002642) may be fused to an immunoglobulin Fc region (see, e.g., SEQ ID NO:154). Also as described herein, the Fc portion of the fusion polypeptide may be derived from a human or non-human immunoglobulin. By way of example, the Fc portion of a 130L-Fc fusion polypeptide may comprise the amino acid sequence of all or a portion of the hinge region, CH2 domain, and CH3 domain of a human immunoglobulin, for example, an IgG1. A 130L-Fc fusion polypeptide may further comprise a signal peptide sequence that facilitates post-translational transport of the polypeptide in the host cell in which the fusion polypeptide is expressed. The signal peptide sequence may be derived from a 130L signal peptide sequence encoded by the poxvirus genome from which the 130L sequence was obtained. Alternatively, the signal peptide sequence may comprise an amino acid sequence that is derived from an unrelated polypeptide, such as human growth hormone.

An Fc polypeptide as described herein also includes Fc polypeptide variants. One such Fc polypeptide variant has one or more cysteine residues (such as one or more cysteine residues in the hinge region) that forms an interchain disulfide bond substituted with another amino acid, such as serine, to reduce the number of interchain disulfide bonds that can form between the two heavy chain constant region polypeptides that form an Fc polypeptide. In addition, or alternatively, the most amino terminal cysteine residue of the hinge region that forms a disulfide bond with a light chain constant region in a complete immunoglobulin molecule may be substituted, for example, with a serine residue. Alternatively, one or more cysteine residues may be deleted from the wildtype hinge of the Fc polypeptide. Another example of an Fc polypeptide variant is a variant that has one or more amino acids involved in an effector function substituted or deleted such that the Fc polypeptide has a reduced level of an effector function. For example, amino acids in the Fc region may be substituted to reduce or abrogate binding of a component of the complement cascade (see, e.g., Duncan et al., Nature 332:563-64 (1988); Morgan et al., Immunology 86:319-24 (1995)) or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity. Such an Fc polypeptide variant that differs from the wildtype Fc polypeptide is also called herein a mutein Fc polypeptide.

In one embodiment, a 130L polypeptide (or fragment or variant thereof) is fused with an Fc polypeptide that comprises at least one substitution of a residue that in the wildtype Fc region polypeptide contributes to binding of an Fc polypeptide or immunoglobulin to one or more IgG Fc receptors expressed on certain immune cells. Such a mutein Fc polypeptide comprises at least one substitution of an amino acid residue in the CH2 domain of the mutein Fc polypeptide, such that the capability of the fusion polypeptide to bind to an IgG Fc receptor, such as an IgG Fc receptor present on the surface of an immune cell, is reduced.

As discussed herein, residues in the amino terminal portion of the CH2 domain that contribute to IgG Fc receptor binding include residues at positions Leu234-Ser239 (Leu-Leu-Gly-Gly-Pro-Ser (SEQ ID NO:152) (EU numbering system, Kabat et al., supra) (see, e.g., Morgan et al., Immunology 86:319-24 (1995), and references cited therein). Substitution of the amino acid at one or more of these six positions (i.e., one, two, three, four, five, or all six) in the CH2 domain results in a reduction of the capability of the Fc polypeptide to bind to one or more of the IgG Fc receptors (or isoforms thereof) (see, e.g., Burton et al., Adv. Immunol. 51:1 (1992); Hulett et al., Adv. Immunol. 57:1 (1994); Jefferis et al., Immunol. Rev. 163:59 (1998); Lund et al., J. Immunol. 147:2657 (1991); Sarmay et al., Mol. Immunol. 29:633 (1992); Lund et al., Mol. Immunol. 29:53 (1992); Morgan et al., supra). In addition to substitution of one or more amino acids at EU positions 234-239, one, two, or three or more amino acids adjacent to this region (either to the carboxy terminal side of position 239 or to the amino terminal side of position 234) may also be substituted.

By way of example, substitution of the leucine residue at position 235 with a glutamic acid residue or an alanine residue abolishes or reduces, respectively, the affinity of an immunoglobulin (such as human IgG3) for FcγRI (Lund et al., 1991, supra; Canfield et al., supra; Morgan et al., supra). As another example, replacement of the leucine residues at positions 234 and 235, for example, with alanine residues, abrogates binding of an immunoglobulin to FcγRIIa (see, e.g., Wines et al., supra). Alternatively, leucine at position 234, leucine at position 235, and glycine at position 237, each may be substituted with a different amino acid, such as leucine at position 234 may be substituted with an alanine residue (L234A), leucine at 235 may be substituted with an alanine residue (L235A) or with a glutamic acid residue (L235E), and the glycine residue at position 237 may be substituted with another amino acid, for example an alanine residue (G237A).

In one embodiment, a mutein Fc polypeptide that is fused in frame to a 130L polypeptide (or variant or fragment thereof) comprises one, two, three, four, five, or six mutations located between positions 15-20 of SEQ ID NO:145 or between positions 13-18 of SEQ ID NO: 146 (substitutions at positions corresponding to EU 234, 235, and 237) that correspond to positions 234-239 of a human IgG1 CH2 domain (EU numbering system) as described herein.

In another embodiment, a mutein Fc polypeptide comprises a mutation of a cysteine residue in the hinge region of an Fc polypeptide. In one embodiment, the cysteine residue most proximal to the amino terminus of the hinge region of an Fc polypeptide (e.g., for example, the cysteine residue most proximal to the amino terminus of the hinge region of the Fc portion of a wildtype IgG1 immunoglobulin) is deleted or substituted with another amino acid. That is, by way of illustration, the cysteine residue at position 1 of SEQ ID NO:145 is deleted, or the cysteine residue at position 1 is substituted with another amino acid that is incapable of forming a disulfide bond, for example, with a serine residue. In another embodiment, a mutein Fc polypeptide comprises a deletion or substitution of the cysteine residue most proximal to the amino terminus of the hinge region of an Fc polypeptide further comprises deletion or substitution of the adjacent C-terminal amino acid. In a certain embodiment, this cysteine residue and the adjacent C-terminal residue are both deleted from the hinge region of a mutein Fc polypeptide. In a specific embodiment, the cysteine residue at position 1 of SEQ ID NO:145 and the aspartic acid at position 2 of SEQ ID NO:145 are deleted. Fc polypeptides that comprise deletion of the most amino terminal cysteine residue in the hinge region are more efficiently expressed in a host cell that comprises a recombinant expression construct encoding such a Fc polypeptide.

In a specific embodiment, a mutein Fc polypeptide comprises the amino acid sequence set forth in SEQ ID NO:146, which differs from the wildtype Fc polypeptide (SEQ ID NO:145) wherein the cysteine residue most proximal to the amino terminus of the hinge region of an Fc polypeptide is deleted and the C-terminal adjacent aspartic acid is deleted and the leucine reside that corresponds to EU234 is substituted with an alanine residue, the leucine residue that corresponds to EU235 is substituted with a glutamic acid residue, and the glycine that corresponds to EU237 is substituted with an alanine residue (see SEQ ID NO:146). Thus, an exemplary mutein Fc polypeptide has an amino acid sequence at its amino terminal end of KTHTCPPCPAPEAEGAPS (SEQ ID NO:148) (positions 1-18 of SEQ ID NO:146).

Other Fc variants encompass similar amino acid sequences of known Fc polypeptide sequences that have only minor changes, for example by way of illustration and not limitation, covalent chemical modifications, insertions, deletions and/or substitutions, which may further include conservative substitutions. Amino acid sequences that are similar to one another may share substantial regions of sequence homology. Similarly, nucleotide sequences that encode the Fc variants may encompass substantially similar nucleotide sequences and have only minor changes, for example by way of illustration and not limitation, covalent chemical modifications, insertions, deletions, and/or substitutions, which may further include silent mutations owing to degeneracy of the genetic code. Nucleotide sequences that are similar to one another may share substantial regions of sequence homology.

An Fc polypeptide or at least one immunogloblulin constant region, or portion thereof, when fused to a peptide or polypeptide of interest acts, at least in part, as a vehicle or carrier moiety that prevents degradation and/or increases half-life, reduces toxicity, reduces immunogenicity, and/or increases biological activity of the peptide such as by forming dimers or other multimers (see, e.g., U.S. Pat. Nos. 6,018,026; 6,291,646; 6,323,323; 6,300,099; 5,843,725). (See also, e.g., U.S. Pat. No. 5,428,130; U.S. Pat. No. 6,660,843; U.S. Patent Application Publication Nos. 2003/064480; 2001/053539; 2004/087778; 2004/077022; 2004/071712; 2004/057953/ 2004/053845/ 2004/044188; 2004/001853; 2004/082039).

A 130L polypeptide (or variant or fragment thereof) fused in frame with an Fc polypeptide or Fc polypeptide variant (e.g., a mutein Fc polypeptide) may comprise a peptide linker between the 130L polypeptide and Fc polypeptide. The linker may be a single amino acid (such as for example a glycine residue) or may be two, three, four, five, six, seven, eight, nine, or ten amino acids, or may be any number of amino acids between 10 and 20 amino acids. By way of illustration but not limitation, a linker may comprise at least two amino acids that are encoded by a nucleotide sequence that is a restriction enzyme recognition site. Examples of such restriction enzyme recognition sites include, for example, BamHI, ClaI, EcoRI, HindIII, KpnI, NcoI, NheI, PmlI, PstI, SalI, and XhoI.

A 130L polypeptide, fragment thereof, or variant thereof, fused in frame with a mutein Fc polypeptide may be used to suppress an immune response in a subject when administered with a pharmaceutically or physiologically suitable carrier or excipient according to methods described herein and known to practitioners in the medical art. Such fusion polypeptides may alter a biological activity of at least one of the RPTP polypeptides described herein (i.e., LAR, RPTP-σ, RPTP-δ), at least two of the RPTP polypeptides or all three RPTP polypeptides. In certain embodiments, a 130L polypeptide, fragment thereof, or variant thereof, fused in frame with a mutein Fc polypeptide is used for treating an immunological disease e or disorder (including an autoimmune disease or an inflammatory disease), which are described in detail herein. As described herein, the 130L/mutein Fc polypeptides may also be used to treat a disease or disorder associated with alteration of cell migration, cell proliferation, or cell differentiation, which includes but is not limited to an immunological disease or disorder, a cardiovascular disease or disorder, a metabolic disease or disorder, or a proliferative disease or disorder.

130L polypeptide fragments include 130L polypeptide variant fragments. 130L polypeptide fragments also include 130L fragments having an amino acid sequence that differs from the full-length 130L from which the fragments were derived, that is the 130L polypeptide fragment variant has at least 99%, 98%, 97%, 95%, 90%, 87%, 85%, or 80% amino acid sequence identity with a portion of the full-length 130L polypeptide. Variants of 130L polypeptide fragments that have the capability to alter (suppress or enhance) the immunoresponsiveness of an immune cell retain comparable capability to alter the immunoresponsiveness of an immune cell.

130L polypeptide variants and 130L polypeptide fragment variants that retain the capability to alter immunoresponsiveness of an immune cell include variants that contain conservative amino acid substitutions. A variety of criteria known to persons skilled in the art indicate whether amino acids at a particular position in a peptide or polypeptide are conservative (or similar). For example, a similar amino acid or a conservative amino acid substitution is one in which an amino acid residue is replaced with an amino acid residue having a similar side chain, such as amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, or the BLAST algorithm, as described herein).

A 130L polypeptide variant also includes a variant that interacts with or binds to only one or two (i.e., LAR and RPTP-δ, LAR and RPTP-σ, or RPTP-δ and RPTP-σ) but not all three of LAR, RPTP-δ, and RPTP-σ. Such a variant comprises at least 1, 2, 3, 4, 5, 6, 7, 8, 9, 10, 11-15, 16-25, 26-35, or 36-45 amino acid substitutions, deletions, or insertions compared with the wildtype 130L polypeptide. Binding of 130L to each of the RPTPs may be determined according to methods described herein and practiced in the art. The source of the polypeptides for binding studies includes, for example, isolated 130L and RPTPs, or fragments thereof, or individual cell lines capable of recombinant expression of one of 130L, LAR, RPTP-δ, and RPTP-σ.

Variants of 130L full-length polypeptides or 130L fragments may be readily prepared by genetic engineering and recombinant molecular biology methods and techniques. Analysis of the primary and secondary amino acid sequence of a 130L polypeptide and computer modeling to analyze the tertiary structure of the polypeptide may aid in identifying specific amino acid residues that can be substituted without altering the structure and as a consequence, potentially the function, of the 130L polypeptide. Modification of DNA encoding a 130L polypeptide or fragment may be performed by a variety of methods, including site-specific or site-directed mutagenesis of the DNA, which methods include DNA amplification using primers to introduce and amplify alterations in the DNA template, such as PCR splicing by overlap extension (SOE). Mutations may be introduced at a particular location by synthesizing oligonucleotides containing a mutant sequence, flanked by restriction sites enabling ligation to fragments of the native sequence. Following ligation, the resulting reconstructed sequence encodes a variant (or derivative) having the desired amino acid insertion, substitution, or deletion.

Site-directed mutagenesis is typically effected using a phage vector that has single- and double-stranded forms, such as an M13 phage vector, which is well-known and commercially available. Other suitable vectors that contain a single-stranded phage origin of replication may be used (see, e.g., Veira et al., Meth. Enzymol. 15:3 (1987)). In general, site-directed mutagenesis is performed by preparing a single-stranded vector that encodes the protein of interest. An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. Additional disclosure relating to site-directed mutagenesis may be found, for example, in Kunkel et al. (Meth. Enzymol. 154:367 (1987)) and in U.S. Pat. Nos. 4,518,584 and 4,737,462. The heteroduplex is introduced into appropriate bacterial cells, and clones that include the desired mutation are selected. The resulting altered DNA molecules may be expressed recombinantly in appropriate host cells to produce the variant, modified protein.

Oligonucleotide-directed site-specific (or segment specific) mutagenesis procedures may be employed to provide an altered polynucleotide that has particular codons altered according to the substitution, deletion, or insertion desired. Deletion or truncation derivatives of proteins may also be constructed by using convenient restriction endonuclease sites adjacent to the desired deletion. Subsequent to restriction, overhangs may be filled in and the DNA religated. Exemplary methods of making the alterations set forth above are disclosed by Sambrook et al. (Molecular Cloning: A Laboratory Manual, 3d ed., Cold Spring Harbor Laboratory Press, NY 2001). Alternatively, random mutagenesis techniques, such as alanine scanning mutagenesis, error prone polymerase chain reaction mutagenesis, and oligonucleotide-directed mutagenesis may be used to prepare 130L polypeptide variants and fragment variants (see, e.g., Sambrook et al., supra).

Assays for assessing whether the variant folds into a conformation comparable to the non-variant polypeptide or fragment include, for example, the ability of the protein to react with mono- or polyclonal antibodies that are specific for native or unfolded epitopes, the retention of ligand-binding functions, and the sensitivity or resistance of the mutant protein to digestion with proteases (see Sambrook et al., supra). 130L variants as described herein can be identified, characterized, and/or made according to these methods described herein or other methods known in the art, which are routinely practiced by persons skilled in the art.

Mutations that are made or identified in the nucleic acid molecules encoding a 130L polypeptide preferably preserve the reading frame of the coding sequences. Furthermore, the mutations will preferably not create complementary regions that when transcribed could hybridize to produce secondary mRNA structures, such as loops or hairpins, that would adversely affect translation of the mRNA. Although a mutation site may be predetermined, the nature of the mutation per se need not be predetermined. For example, to select for optimum characteristics of a mutation at a given site, random mutagenesis may be conducted at the target codon and the expressed mutants screened for gain or loss or retention of biological activity.

A 130L polynucleotide is any polynucleotide that encodes a 130L polypeptide or at least a portion (or fragment) of a 130L polypeptide or a variant thereof, or that is complementary to such a polynucleotide. The nucleotide sequences of polynucleotides that encode 130L, or its orthologues, may be found, for example, in the genomic sequences of yatapoxviruses provided in GenBank entries for which Accession numbers are provided herein, in GenBank Accession Nos. AJ293568 and NC_{—}002642 and that can be deduced from the amino acid sequences disclosed herein (e.g., SEQ ID NO:85 and SEQ ID NO:150). Polynucleotides comprise at least 15 consecutive nucleotides or at least 30, 35, 40, 50, 55, or 60 consecutive nucleotides, in certain embodiments at least 70, 75, 80, 90, 100, 110, 120, 125, or 130 consecutive nucleotides, and in other embodiments at least 135, 140, 145, 150, 155, 160, or 170 consecutive nucleotides, and in other embodiments at least 180, 190, 200, 225, 250, 275, 300, 325, 350, 375, 400, 405, 410, 420, 425, 445, 450, 475, 500, 525, 530, 545, 550, 575, 600, 625, 650, or 660 consecutive nucleotides that include sequences encoding a 130L polypeptide, or nucleotide sequences that are complementary to such a sequence. Certain polynucleotides that encode a 130L polypeptide, variant, or fragment thereof may also be used as probes, primers, short interfering RNA (siRNA), or antisense oligonucleotides, as described herein. Polynucleotides may be single-stranded DNA or RNA (coding or antisense) or double-stranded RNA (e.g., genomic or synthetic) or DNA (e.g., cDNA or synthetic).

Polynucleotide variants may also be identified by hybridization methods. Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5×, and 0.2×SSC containing 0.05-0.1% SDS. For additional stringency, conditions may include a wash in 0.1×SSC and 0.1% SDS at 50-60° C. for 15 minutes. As understood by persons having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used (i.e., for example, the guanine plus cytosine (G/C) versus adenine plus thymidine (A/T) content). Accordingly, a person skilled in the art will appreciate that suitably stringent conditions can be readily selected without undue experimentation when a desired selectivity of the probe is identified.

### Receptor Protein Tyrosine Phosphatases (RPTP): LAR, RPTP-δ, and RPTP-σ

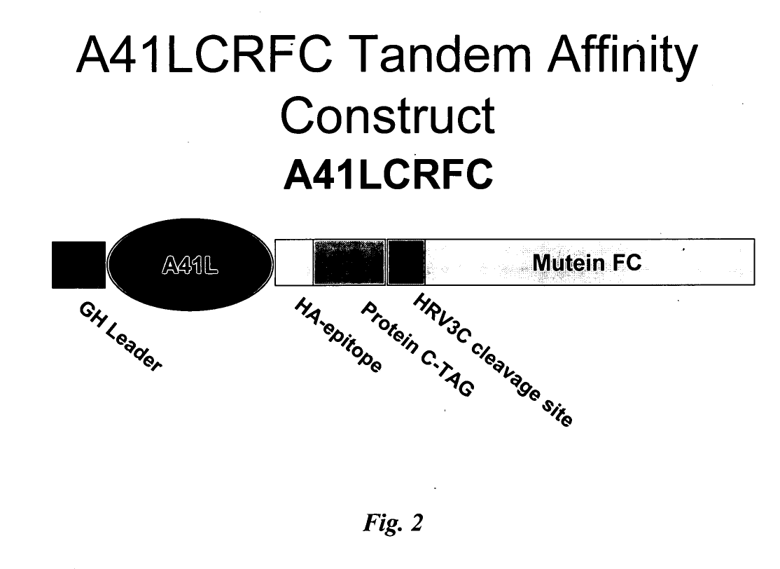

The leukocyte common-antigen-related protein (LAR), receptor-like protein tyrosine phosphatase-δ (RPTP-δ), and RPTP-σ are members of the receptor-like type II protein tyrosine phosphatases (PTPs). These RPTPs (also referred to herein as protein tyrosine phosphatases (PTP) or receptor protein tyrosine phosphatases) have three immunoglobulin-like (Ig-like) domains, a series of fibronectin type III-like motifs in the extracellular domain, a potential proteolytic processing site, a transmembrane element, and two tandem cytoplasmic phosphatase domains D1 and D2 (see, e.g., Alonso et al., Cell 117:699-711 (2004), see FIG. 2 therein; Streuli et al., J. Exp. Med. 168:1523 (1988); Charbonneau et al., Annu. Rev. Cell Biol. 8:463-93 (1992); Pan et al., J. Biol. Chem. 268:19284-91 (1993); Walton et al., Neuron 11:387-400 (1993); Yan et al., J. Biol. Chem. 268:24880-86 (1993); Zhang et al., Biochem. J. 302:39-47 (1994); Pulido et al., J. Biol. Chem. 270:6722-28 (1995)).

Several alternatively spliced variants of LAR have been identified, and are believed to be developmentally regulated (O'Grady et al., J. Biol. Chem. 269:25193 (1994); Zhang and Longo, J. Cell. Biol. 128:415 (1995); Honkaniemi et al., Mol. Brain. Res. 61:1 (1998)). Multiple isoforms of RPTP-δ and RPTP-σ as well as LAR appear to be generated by tissue-specific alternative splicing (see, e.g., Pulido et al., Proc. Natl. Acad. Sci. USA 92:11686-90 (1995)). In humans, the LAR gene maps to chromosome 1p32, a region that is frequently deleted in tumors of neuroectodermal origin (Jirik et al., Cytogenet. Cell Genet. 61:266 (1992)).

Protein tyrosine phosphatases such as LAR, RPTP-δ, and RPTP-σ dephosphorylate tyrosyl phosphoproteins that are components of cellular signal transduction pathways. Regulated phosphorylation and dephosphorylation of tyrosine residues of substrates is a major control mechanism for cellular processes such as cell growth, cell proliferation, metabolism, differentiation, and locomotion. Accordingly, the activities of protein tyrosine phosphatases and protein tyrosine kinases that regulate reversible tyrosine phosphorylation must be integrated and regulated in a cell. Abnormal regulation results in manifestation of several diseases and disorders. (See, e.g., Tonks and Neel, Curr. Opin. Cell Biol. 13:182-95 (2001)). Without wishing to be bound by theory, the biological specificity of receptor PTPs (RPTPs) may be derived from their cognate ligands. Certain diverse biological functions of LAR, RPTP-δ, and RPTP-σ have been suggested by the results of gene knockout animal studies. Disruption of expression of the LAR gene results in defective mammary gland development due to impaired terminal differentiation of alveoli during pregnancy (Schaapveid et al., Dev. Biol. 188:134-46 (1996)); some defects in forebrain size and hippocampal organization (Yeo et al., J. Neurosci. Res. 47:348-60 (1997)); and possibly, defects in glucose metabolism (Ren et al., Diabetes 47:493-97 (1998)). By contrast, deletion of RPTP-δ affects hippocampal long-term potentiation and learning (Ren et al., EMBO J. 19:2775-85 (2000)), and RPTP-σ deficient mice exhibit defects in brain development, including reduction in the size of the hypothalamus, olfactory bulb, and pituitary gland (Elchebly et al., Nat. Genet. 21:330-33 (1999); Wallace et al., Nat. Genet. 21:334-38 (1999)).

The results of various studies have suggested a number of biological roles for LAR: altering ability of cells to proliferate (see, e.g., Yang et al., Carcinogenesis 21:125; Tisi et al., J. Neurobiol. 42:477 (2000)); suppressing tumor cell growth (Zhai et al., Mol. Carcinogen. 14:103 (1995)); dephosphorylating the insulin receptor and affecting glucose homeostasis (Ahmad and Goldstein, J. Biol. Chem. 272:448 (1997); Ren et al., Diabetes 47:493 (1998)); regulating cell-matrix interactions (Pulido et al., supra); regulating synapse morphogenesis and function (see, e.g., Dunah et al., Nat. Neurosci. 8:458-67 (2005); and affecting immune cell function (U.S. Pat. No. 6,852,486). While studies have indicated that RPTP-δ and RPTP-σ may also affect cell adhesion (Pulido et al., supra) and synapse morphogenesis and function (see, e.g., Dunah et al., supra), none have suggested that these two phosphatases may also affect immune cell function. Accordingly, embodiments described herein relate to the unexpected discovery that all three phosphatases, LAR, RPTP-δ, and RPTP-σ are expressed by immune cells.

LAR, RPTP-δ, and RPTP-σ are cellular targets of the viral proteins A41L and 130L. Binding of these viral proteins to any one of these phosphatases can affect immune cell function. Particularly, A41L or 130L may suppress an immune response and act as a suppressor of the host immune system. Exemplary isoforms of LAR to which A41L and 130L may bind and alter the function include LAR comprising an amino acid sequence set forth in GenBank Accession Nos. NP_{—}002832 (SEQ ID NO:22) (encoded by a polynucleotide having the nucleotide sequence set forth in NM_{—}002840 (SEQ ID NO:23)); SEQ ID NO:24 (AAH48768) (encoded by a polynucleotide having the nucleotide sequence set forth in BCO48768 (SEQ ID NO:65)); CAI14894 (SEQ ID NO:25); GenBank NP_{—}569707 (SEQ ID NO:26) (encoded by a polynucleotide having the nucleotide sequence set forth in NM_{—}130440 (SEQ ID NO:27)); and CAI14895 (SEQ ID NO:28). Exemplary isoforms of RPTP-σ to which A41L or 130L may bind and alter the function include RPTP-σ comprising an amino acid sequence set forth in GenBank NP_{—}002841 (SEQ ID NO:29) (encoded by a polynucleotide having the nucleotide sequence set forth in NM_{—}002850 (SEQ ID NO:30)); NP_{—}570924 (SEQ ID NO:31) (encoded by a polynucleotide having the nucleotide sequence set forth in NM_{—}130854 (SEQ ID NO:32)); GenBank NP_{—}570923 (SEQ ID NO:33) (encoded by a polynucleotide having the nucleotide sequence set forth in NM_{—}130853 (SEQ ID NO:34)); and NP_{—}570925 (SEQ ID NO:35) (encoded by a polynucleotide having the nucleotide sequence set forth in NM_{—}130855 (SEQ ID NO:36)); and Q13332 (SEQ ID NO:64)). Exemplary isoforms of RPTP-δ to which a viral protein may bind and alter the function include RPTP-δ comprising an amino acid sequence set forth in GenBank NP_{—}002830 (SEQ ID NO:37) (encoded by a polynucleotide having the nucleotide sequence set forth in NM_{—}002839 (SEQ ID NO:38)); NP_{—}569075 (SEQ ID NO:39) (encoded by a polynucleotide having the nucleotide sequence set forth in NM_{—}120391 (SEQ ID NO:40)); NP_{—}569076 (SEQ ID NO:41) (encoded by a polynucleotide having the nucleotide sequence set forth in NM_{—}130392 (SEQ ID NO:42)); and NP_{—}569077 (SEQ ID NO:43) (encoded by a polynucleotide having the nucleotide sequence set forth in NM_{—}130393 (SEQ ID NO:44)).

The LAR, RPTP-δ, and RPTP-σ polypeptides described herein also include variants or each respective RPTP, and which have a similar amino acid sequence to the amino acid sequences disclosed herein. Variants include, for example, naturally occurring polymorphisms (e.g., such as allelic variants) or recombinantly manipulated or engineered RPTP polypeptide variants. An RPTP variant has at least 70%, 75%, 80%, 85%, 90%, 95%, or 98% identity or similarity to the wild-type RPTP. A variety of criteria known to persons skilled in the art indicate whether amino acids at a particular position in a peptide or polypeptide are conservative or similar. For example, a similar amino acid or a conservative amino acid substitution is one in which an amino acid residue is replaced with an amino acid residue having a similar side chain, such as amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. The percent identity or similarity between two RPTPs having an amino acid sequence can be readily determined by alignment methods (e.g., using GENEWORKS, Align or the BLAST algorithm), which are also described herein and are familiar to a person having ordinary skill in the art.

An RPTP variant may also be readily prepared by genetic engineering and recombinant molecular biology methods and techniques as described herein regarding A41L polypeptide variants. Briefly, analysis of the primary and secondary amino acid sequence of an RPTP and computer modeling to analyze the tertiary structure of the polypeptide may aid in identifying specific amino acid residues that can be substituted. Modification of DNA encoding an RPTP polypeptide or fragment may be performed by a variety of methods, including site-specific or site-directed mutagenesis of the DNA, which methods include DNA amplification using primers to introduce and amplify alterations in the DNA template, such as PCR splicing by overlap extension (SOE). Mutations may be introduced at a particular location by synthesizing oligonucleotides containing a mutant sequence, flanked by restriction sites enabling ligation to fragments of the native sequence. Following ligation, the resulting reconstructed sequence encodes a variant (or derivative) having the desired amino acid insertion, substitution, or deletion.

As described herein site-directed mutagenesis is typically effected using a phage vector that has single- and double-stranded forms, such as an M13 phage vector, which is well known and commercially available (see, e.g., Veira et al., Meth. Enzymol. 15:3 (1987); Kunkel et al., Meth. Enzymol. 154:367 (1987)) and in U.S. Pat. Nos. 4,518,584 and 4,737,462). Oligonucleotide-directed site-specific (or segment specific) mutagenesis procedures may be employed to provide an altered polynucleotide that has particular codons altered according to the substitution, deletion, or insertion desired. Deletion or truncation derivatives of proteins may also be constructed by using convenient restriction endonuclease sites adjacent to the desired deletion. Exemplary methods of making the alterations set forth above are disclosed by Sambrook et al. (Molecular Cloning: A Laboratory Manual, 3d ed., Cold Spring Harbor Laboratory Press, NY 2001). Alternatively, random mutagenesis techniques, such as alanine scanning mutagenesis, error prone polymerase chain reaction mutagenesis, and oligonucleotide-directed mutagenesis may be used to prepare RPTP polypeptide variants and fragment variants (see, e.g., Sambrook et al., supra). Assays for assessing whether the variant folds into a conformation comparable to the non-variant polypeptide or fragment include, for example, the ability of the protein to react with mono- or polyclonal antibodies that are specific for native or unfolded epitopes, the retention of ligand-binding functions, and the sensitivity or resistance of the mutant protein to digestion with proteases (see Sambrook et al., supra). RPTP variants as described herein can be identified, characterized, and/or made according to these methods described herein or other methods known in the art, which are routinely practiced by persons skilled in the art.

Mutations that are made or identified in the nucleic acid molecules encoding an RPTP polypeptide preferably preserve the reading frame of the coding sequences. Furthermore, the mutations will preferably not create complementary regions that when transcribed could hybridize to produce secondary mRNA structures, such as loops or hairpins, that would adversely affect translation of the mRNA. Although a mutation site may be predetermined, the nature of the mutation per se need not be predetermined. For example, to select for optimum characteristics of a mutation at a given site, random mutagenesis may be conducted at the target codon and the expressed mutants screened for gain or loss or retention of biological activity.

An RPTP variant retains at least one biological activity or function (e.g., phosphatase activity, mediate or initiate a signal transduction event associated with the wildtype RPTP, bind to at least one cognate ligand, and as further described in detail herein) of the wildtype RPTP. Preferably, the RPTP retains the capability to interact with its cognate ligand(s) and to dephosphorylate a tyrosine phosphorylated substrate.

Polynucleotide variants may also be identified by hybridization methods. Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5×, and 0.2×SSC containing 0.05-0.1% SDS. For additional stringency, conditions may include a wash in 0.1×SSC and 0.1% SDS at 50-60° C. for 15 minutes. As understood by persons having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used (i.e., for example, the guanine plus cytosine (G/C) versus adenine plus thymidine (A/T) content). Accordingly, a person skilled in the art will appreciate that suitably stringent conditions can be readily selected without undue experimentation when a desired selectivity of the probe is identified.

Each of the RPTPs has a signal peptide sequence of approximately 20-30 amino acids at the amino terminal end (see, e.g., Pulido et al. supra) (see also, e.g., the GenBank database reports). Signal peptides are not exposed on the cell surface of a secreted or transmembrane protein because either the signal peptide is cleaved during translocation of the protein or the signal peptide remains anchored in the outer cell membrane (such a peptide is also called a signal anchor) (see, e.g., Nielsen et al., Protein Engineering 10:1-6 (1997); Nielsen et al., in J. Glasgow et al., eds., Proc. Sixth Int. Conf. on Intelligent Systems for Molecular Biology, 122-30 (AAAI Press 1998)). Accordingly, the signal peptide sequence of an RPTP would likely not be part of a binding site on the extracellular portion of the RPTP to which a ligand would bind, such as A41L or an antibody or antigen-binding fragment thereof that specifically binds to the extracellular portion of the RPTP.

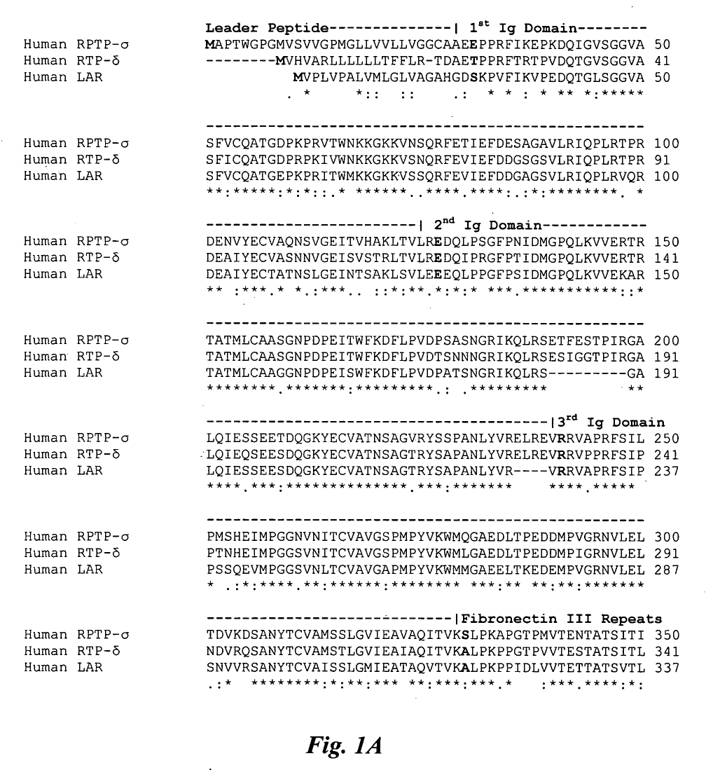

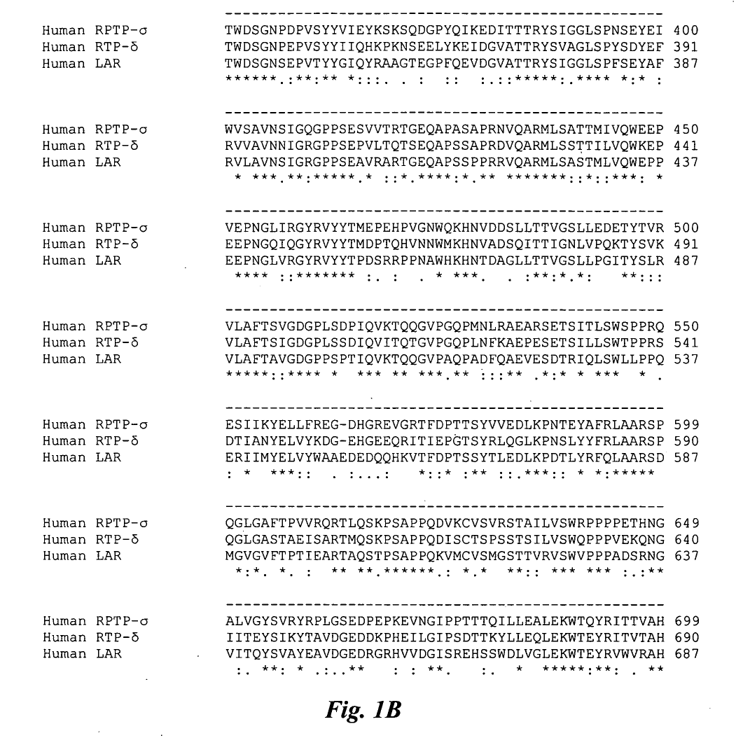

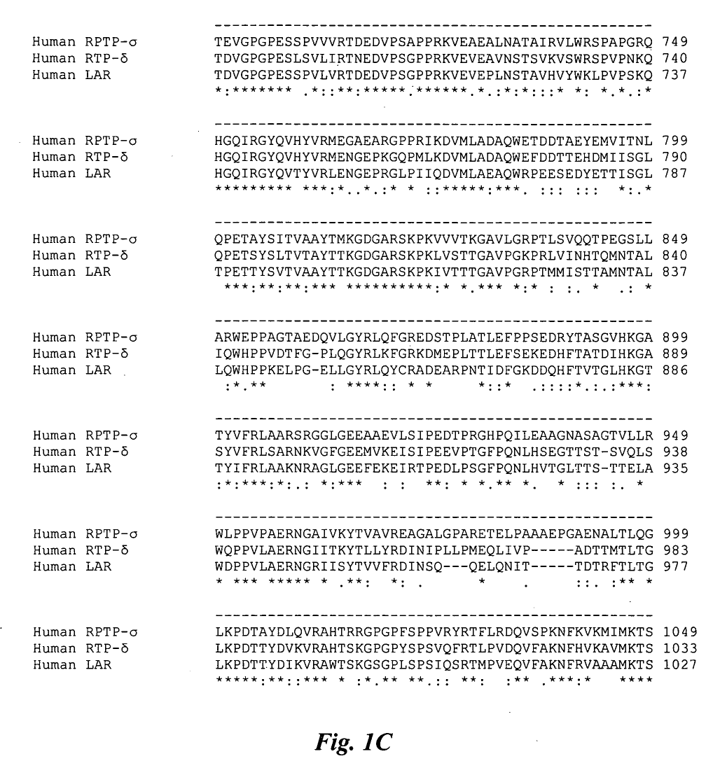

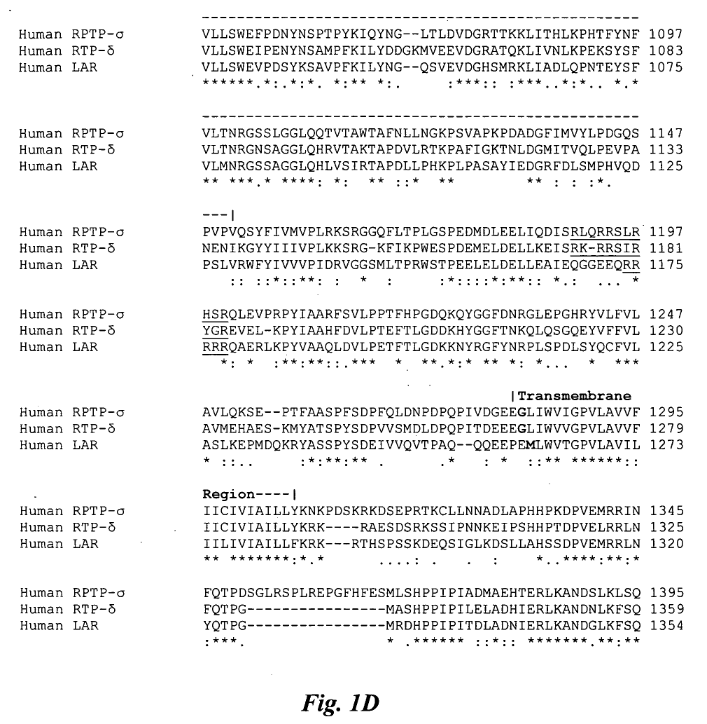

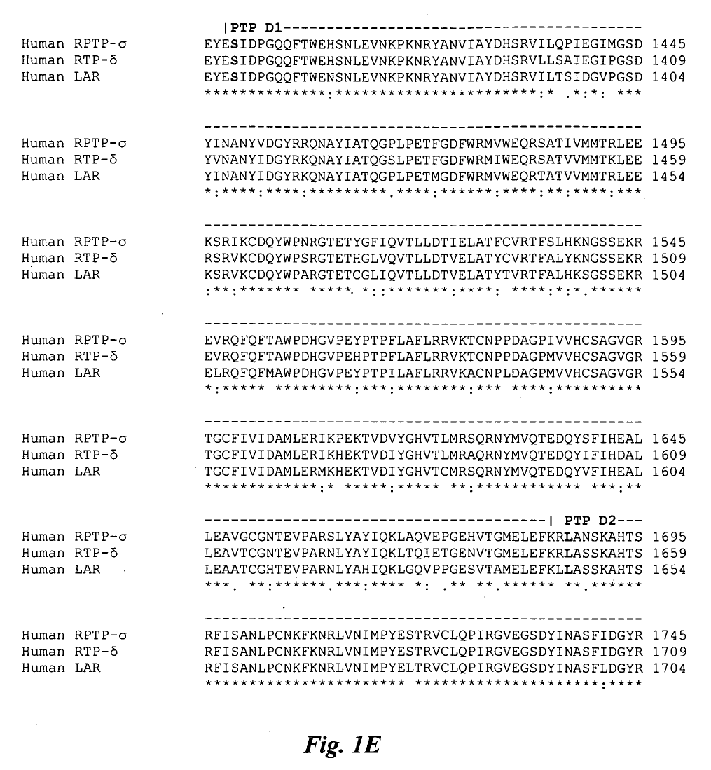

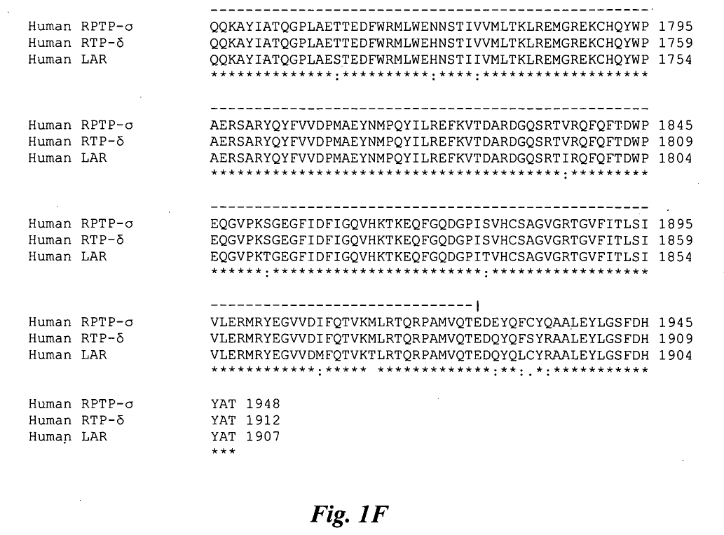

As described herein, the extracellular portion of the RPTP that is exposed on the outer surface of a cell (such as an immune cell), which does not include the signal peptide (also referred to herein as the mature RPTP), comprises three immunoglobulin-like domain(s). The immunoglobulin domains (or immunoglobulin-like domains) are referred to herein as the first, second, and third immunoglobulin domains (alternatively, referred to as Ig-1, Ig-2, Ig-3 or as immunoglobulin-like domain 1, immunoglobulin-like domain 2, and immunoglobulin-like domain 3), wherein the first immunoglobulin domain is the domain that is most proximal to the N-terminus of the RPTP (see FIG. 1). The first immunoglobulin domain is immediately adjacent to the carboxy end of the signal peptide (see FIG. 1). Thus, as used herein, the first immunoglobulin-like domain of an RPTP is the immunoglobulin-like domain that is most proximal to the amino terminus of the RPTP; the second immunoglobulin-like domain of an RPTP is the immunoglobulin-like domain that is between the first and third immunoglobulin-like domains of an RPTP; and the third immunoglobulin-like domain of an RPTP is the immunoglobulin-like domain that is most proximal to the carboxy terminus of the RPTP.

A person skilled in the protein art will understand that the termini or boundaries of the domains do not necessarily correspond to exact amino acid positions in the primary sequence as shown, for example, in FIG. 1. Accordingly, for example, the immunoglobulin domains, fibronectin III repeats, and the catalytic domains may include one, two, three, four, five, six, seven, eight, or more amino acids at positions adjacent to the amino-terminal end and/or the carboxy terminal end of each domain. A person skilled in the art can readily determine what positions in an RPTP correspond to each of the Ig-like domains of the RPTP using the sequences and figures provided herein and the sequences known in the art (both amino acid and the encoding nucleotide sequence). For example, but not limiting, the Ig-1 domain of LAR corresponds to amino acid positions 31-125 of SEQ ID NO:25; the Ig-2 domain of LAR corresponds to amino acid positions 111-227; and the Ig-3 domain of LAR corresponds to amino acid positions 228-316. For RPTP-σ, the Ig-1 domain corresponds to amino acid positions 31-125; the Ig-2 domain corresponds to amino acid positions 127-240; and the Ig-3 domain corresponds to amino acid positions 241-329. For RPTP-δ, the Ig-1 domain corresponds to amino acid positions 22-116; the Ig-2 domain corresponds to amino acid positions 118-231; and the Ig-3 domain corresponds to amino acid positions 232-320. As discussed herein, the amino acids at each terminal end of the domains may vary depending upon the particular RPTP, or variant thereof (such as an allelic variant, cell type variant, or the like), a Ig domain variant includes an Ig domain of the LAR, RPTP-δ, or RPTP-σ that is 99%, 98%, 97%, 96%, 95%, or 90%, 85%, or 80% identical to the sequences for each immunoglobulin-like domain of each RPTP described herein.

In one embodiment, the extracellular portion of LAR, RPTP-δ, or RPTP-σ may be used to alter (enhance or suppress in a statistically or biologically significant manner) the immunoresponsiveness of an immune cell. In another embodiment, an extracellular portion of an RPTP (also referred to herein as soluble LAR, RPTP-δ, or RPTP-σ) that comprises at least one, two or all three of the immunoglobulin-like domains of LAR, RPTP-δ, or RPTP-σ and does not include any one or more of the fibronectin domains of the RPTP may be used to alter the immunoresponsiveness of an immune cell. For ease of reference, the latter polypeptides (i.e., an RPTP (LAR, RPTP-δ, or RPTP-σ) that comprises at least one, two or all three of the immunoglobulin-like domains, as a monomer or oligomers as described herein) are referred to herein as RPTP Ig-like domain polypeptides.

In certain embodiments, the immunoresponsiveness of an immune cell is enhanced. The extracellular portion or fragment of the RPTP, such as the at least one, two or all three immunoglobulin-like domain(s), can be administered to a host or subject such that at least one ligand that binds to the RPTP expressed on an immune cell binds to the exogenously added RPTP fragment. The ligand may be soluble or the ligand may be expressed on the cell surface of the same cell as the immune cell that expresses the RPTP, or the ligand may be a cell surface protein that is expressed by another cell. Thus, a soluble LAR, RPTP-δ, or RPTP-σ may interact with the ligand and reduce the amount of the ligand available to bind to the RPTP expressed on an immune cell, that is, the ligand is blocked from binding to the RPTP expressed on the cell, in turn inhibiting, preventing, diminishing, reducing, or abrogating, the function, activity (e.g., phosphatase activity), or signaling event associated with binding of the ligand to the RPTP.

In another embodiment, an extracellular portion (e.g., at least one, two or all three of the immunoglobulin-like domains) of any one of LAR, RPTP-δ, or RPTP-σ may suppress an immune response. A ligand, which may be either a soluble ligand or a ligand that is a cell surface protein, may interact with an RPTP on the cell surface of an immune cell, and this interaction may induce an inflammatory response or may induce the expression or production of a cytokine (e.g., but not limited to, cytokines described herein including IFN-γ) that induces or exacerbates an inflammatory or autoimmune response. The interaction of one or more of the LAR, RPTP-δ, and RPTP-σ expressed on an immune cell with such a ligand (soluble or a cell surface protein) may be inhibited, prevented, or blocked by soluble RPTP that first interacts with or binds to the ligand.

In a certain embodiment, at least one, or at least two, or all three of the immunoglobulin-like domains are linked (i.e., attached or fused) to a non-RPTP moiety. The moiety may be linked to the RPTP fragment by covalent or noncovalent attachment of the moiety to the fragment, for example, by using conjugation methods, which vary depending on the nature of the moiety (such as if the moiety is a carbohydrate or a polypeptide or small molecule), and with which persons skilled in the particular art are familiar. Alternatively, when the non-RPTP moiety is a peptide or polypeptide, the moiety may be linked recombinantly to form a RPTP fragment fusion polypeptide. For example, recombinant expression constructs may be prepared that comprise a polynucleotide encoding a fusion polypeptide comprising at least one, at least two, or all three immunoglobulin-like domains (or a portion thereof) of the RPTP fused with, for example, an at least one immunoglobulin (Ig) constant region domain or at least two Ig constant region domains of an immunoglobulin Fc polypeptide.

In one embodiment, the second and third immunoglobulin-like domains of LAR, of RPTP-δ, or of RPTP-σ are fused to an immunoglobulin Fc polypeptide; and in still another embodiment, the first, second, and third immunoglobulin like domains of LAR, or of RPTP-δ, or of RPTP-σ are fused to an immunoglobulin Fc polypeptide. In certain embodiments, the first immunoglobulin-like domain of LAR, RPTP-δ, or RPTP-σ is fused to an immunoglobulin Fc polypeptide. In another embodiment, the second immunoglobulin like domain of LAR, RPTP-δ, or RPTP-σ is fused to an immunoglobulin Fc polypeptide; in still another embodiment, the third immunoglobulin like domain of LAR, RPTP-δ, or RPTP-σ is fused to an immunoglobulin Fc polypeptide. In other embodiments, the first and second immunoglobulin like domains of LAR, of RPTP-δ, or of RPTP-σ are fused to an immunoglobulin Fc polypeptide; in yet other embodiments, the first and third immunoglobulin like domains of LAR, of RPTP-δ, or of RPTP-σ are fused to an immunoglobulin Fc polypeptide. In certain instances, use of the first immunoglobulin-like domain alone (i.e., in the absence of the second and/or third immunoglobulin-like domains) or a polypeptide having the first immunoglobulin-like domain and the second immunoglobulin-like domain (i.e., in the absence of the third Ig-like domain) fused to an Fc polypeptide may be less effective to suppress an immune response in an immune cell or in a host in a manner similar to A41L. Without wishing to be bound by any particular theory, and as described herein, because A41L does not bind to the first immunoglobulin-like domain alone in the absence of the second and third Ig-like domains, a RPTP Ig-like domain that incorporates only the first domain may be less effective to interact with a ligand or cell surface polypeptide to effect suppression of an immune response in the same manner as A41L.

In still other embodiments, a soluble RPTP (i.e., a RPTP Ig-like domain polypeptide) may comprise one, two, or three immunoglobulin-like domains in the various combinations described above that is not attached or fused to a non-RPTP moiety. For example, a RPTP Ig-like domain polypeptide may comprise the first, second, and third Ig-like domains of an RPTP (LAR, RPTP-δ, or RPTP-σ); the second and third Ig-like domains of an RPTP. In certain alternative embodiments, a RPTP Ig-like domain polypeptide may comprise the first and second or first and third Ig-like domains of an RPTP; or each Ig-like domain alone.

Soluble RPTP Ig-like domain polypeptides may also exist as multimers, such as dimers and trimers. The multimers may form by noncovalent interactions under conditions that favor such interactions (which include physiological conditions) or may form by a combination of covalent and non-covalent interactions. Alternatively, multimers may be formed by chemically or recombinantly linking at least two monomeric RPTP Ig-like domain polypeptides. The multimers may comprise, for example, homodimers or heterodimers. For instance, a homodimer may comprise (1) a first monomer of at least one, two, or three immunoglobulin-like domains of an RPTP and (2) a second monomer of the same at least one, two, or three immunoglobulin-like domains of the same RPTP. In certain specific embodiments, for example, a homodimer may comprise a first and second monomer that each comprises the second and third (or, alternatively, the first, second, and third) immunoglobulin-like domains of LAR. In another embodiment, each monomer (e.g., the second and third immunoglobulin-like domains or the first, second, and third immunoglobulin-like domains) of a homodimer is derived from RPTP-δ, and in another embodiment, each monomer is derived from RPTP-σ.

Alternatively, the oligomers, such as dimers, may be heterodimers, and each monomer is derived from a different RPTP (i.e., LAR, RPTP-δ, or RPTP-σ). In a certain embodiment, a heterodimer may comprise a first monomer, which includes the second and third (or, alternatively, the first, second, and third) immunoglobulin-like domains of LAR and a second monomer, which includes the second and third (or, alternatively, the first, second, and third) immunoglobulin-like domains, of either RPTP-δ or RPTP-σ. In another embodiment, a first monomer of a heterodimer comprises the second and third (or, alternatively, the first, second, and third) immunoglobulin-like domains of RPTP-δ, and the second monomer of the heterodimer includes the corresponding immunoglobulin-like domains of RPTP-σ.

In certain other embodiments, homodimers or heterodimers comprise a first and second monomer and each monomer comprises only one immunoglobulin-like domain from an RPTP. In still other embodiments, each monomer of a homodimer or a heterodimer comprises the first and third immunoglobulin-like domains of an RPTP; and in certain other embodiments, each monomer comprises the first and second immunoglobulin-like domains of an RPTP. Thus a homodimer may comprise two monomers, each composed of the first and second immunoglobulin-like domains of LAR, or each monomer may be composed of the first and third immunoglobulin-like domains of LAR. Homodimers may be similarly constructed for each of RPTP-δ and RPTP-σ. Heterodimers may be prepared from a first and second monomer, which are different, for example, a first monomer may comprise the first and second immunoglobulin-like domains or first and third immunoglobulin like domains of LAR and the second monomer may comprise the first and second immunoglobulin-like domains or first and third immunoglobulin like domains, respectively of either RPTP-δ or RPTP-σ. In other embodiments, heterodimers may comprise a first monomer comprising the first and second immunoglobulin-like domains, or first and third immunoglobulin like domains, of RPTP-δ and the second monomer may comprise the first and second immunoglobulin-like domains, or first and third immunoglobulin like domains, respectively, of RPTP-σ.

In other embodiments, an immunoglobulin-like domain from one RPTP may be fused to an immunoglobulin domain from a different RPTP. For example, the first immunoglobulin like domain of RPTP-δ or RPTP-σ may be fused to the second and third immunoglobulin-like domains of LAR. A number of combinations of immunoglobulin-like domains from each of the three RPTPs described herein may be envisioned to provide a soluble RPTP molecule that comprises in total two or three immunoglobulin-like domains. As described above, the soluble RPTP Ig domain polypeptides may be prepared recombinantly using molecular biology techniques or may be noncovalently combined or covalently fused with or without one or more linking or spacer amino acids.

An Fc polypeptide of an immunoglobulin that may be fused to a RPTP Ig-like domain polypeptide, as discussed in detail above, comprises the heavy chain CH2 domain and CH3 domain and a portion of or the entire hinge region that is located between CH1 and CH2. Historically, the Fc fragment was derived by papain digestion of an immunoglobulin and included the hinge region of the immunoglobulin. Fc regions are monomeric polypeptides that may be linked into dimeric or multimeric forms by covalent (e.g., particularly disulfide bonds) and non-covalent association. The number of intermolecular disulfide bonds between monomeric subunits of Fc polypeptides varies depending on the immunoglobulin class (e.g., IgG, IgA, IgE) or subclass (e.g., human IgG1, IgG2, IgG3, IgG4, IgA1, IgA2).

Fragments of an Fc polypeptide, such as an Fc polypeptide that is truncated at the C-terminal end (that is at least 1, 2, 3, 4, 5, 10, 15, 20, or more amino acids have been removed or deleted), also may be employed. In certain embodiments, the Fc polypeptides described herein contain multiple cysteine residues, such as at least some or all of the cysteine residues in the hinge region, to permit interchain disulfide bonds to form between the Fc polypeptide portions of two separate RPTP domain(s)/Fc fusion proteins, thus forming RPTP domain(s)/Fc fusion polypeptide dimers. In other embodiments, if retention of antibody dependent cell-mediated cytotoxicity (ADCC) and complement fixation (and associated complement associated cytotoxicity (CDC)) is desired, the Fc polypeptide comprises substitutions or deletions of cysteine residues in the hinge region such that an Fc polypeptide fusion protein is monomeric and fails to form a dimer (see, e.g., U.S. Patent Application Publication No. 2005/0175614).

The Fc portion of the immunoglobulin mediates certain effector functions of an immunoglobulin. Three general categories of effector functions associated with the Fc region include (1) activation of the classical complement cascade, (2) interaction with effector cells, and (3) compartmentalization of immunoglobulins. Presently, an Fc polypeptide, and any one or more constant region domains, and fusion proteins comprising at least one immunoglobulin constant region domain can be readily prepared according to recombinant molecular biology techniques with which a skilled artisan is quite familiar.

An Fc polypeptide is preferably prepared using the nucleotide sequence and the encoded amino acid sequence derived from the animal species for whose use the peptide-IgFc fusion polypeptide is intended. In one embodiment, the Fc polypeptide is of human origin and may be from any of the immunoglobulin classes, such as human IgG1 and IgG2.

An Fc polypeptide as described herein also includes Fc polypeptide variants. One such Fc polypeptide variant has one or more cysteine residues (such as one or more cysteine residues in the hinge region) that forms a disulfide bond with another Fc polypeptide substituted with another amino acid, such as serine, to reduce the number of disulfide bonds formed between two Fc polypeptides. Alternatively, one or more cysteine residues may be deleted from the wildtype hinge of the Fc polypeptide.

Another example of an Fc polypeptide variant is a variant that has one or more amino acids involved in an effector function substituted or deleted such that the Fc polypeptide has a reduced level of an effector function. For example, amino acids in the Fc region may be substituted to reduce or abrogate binding of a component of the complement cascade (see, e.g., Duncan et al., Nature 332:563-64 (1988); Morgan et al., Immunology 86:319-24 (1995)) or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity. Such an Fc polypeptide variant that differs from the wildtype Fc polypeptide is also called herein a mutein Fc polypeptide.

In one embodiment, at least one immunoglobulin like domain of an RPTP (LAR, RPTP-δ, RPTP-σ, or variant thereof) is fused in frame with an Fc polypeptide that comprises at least one substitution of a residue that in the wildtype Fc region polypeptide contributes to binding of an Fc polypeptide or immunoglobulin to one or more IgG Fc receptors expressed on certain immune cells. Such a mutein Fc polypeptide comprises at least one substitution of an amino acid residue in the CH2 domain of the mutein Fc polypeptide, such that the capability of the fusion polypeptide to bind to an IgG Fc receptor, such as an IgG Fc receptor present on the surface of an immune cell, is reduced. The types of Fc IgG receptors expressed on human leukocytes are described in detail above.

As described in detail herein, residues of the amino terminal portion of the CH2 domain that contribute to IgG Fc receptor binding include residues at positions Leu234-Ser239 (Leu-Leu-Gly-Gly-Pro-Ser (SEQ ID NO:80) (EU numbering system, Kabat et al., supra) (see, e.g., Morgan et al., Immunology 86:319-24 (1995), and references cited therein). These positions correspond to positions 15-20 of the amino acid sequence of a human IgG1 Fc polypeptide (SEQ ID NO:79). Substitution of the amino acid at one or more of these six positions (i.e., one, two, three, four, five, or all six) in the CH2 domain results in a reduction of the capability of the Fc polypeptide to bind to one or more of the IgG Fc receptors (or isoforms thereof) (see, e.g., Burton et al., Adv. Immunol. 51:1 (1992); Hulett et al., Adv. Immunol. 57:1 (1994); Jefferis et al., Immunol. Rev. 163:59 (1998); Lund et al., J. Immunol. 147:2657 (1991); Sarmay et al., Mol. Immunol. 29:633 (1992); Lund et al., Mol. Immunol. 29:53 (1992); Morgan et al., supra). In addition to substitution of one or more amino acids at EU positions 234-239, one, two, or three or more amino acids adjacent to this region (either to the carboxy terminal side of position 239 or to the amino terminal side of position 234) may also be substituted.

By way of example, substitution of the leucine residue at position 235 (which corresponds to position 16 of SEQ ID NO:79) with a glutamic acid residue or an alanine residue abolishes or reduces, respectively, the affinity of an immunoglobulin (such as human IgG3) for FcγRI (Lund et al., 1991, supra; Canfield et al., supra; Morgan et al., supra). As another example, replacement of the leucine residues at positions 234 and 235 (which correspond to positions 15 and 16 of SEQ ID NO:79), for example, with alanine residues, abrogates binding of an immunoglobulin to FcγRIIa (see, e.g., Wines et al., supra). Alternatively, leucine at position 234 (which corresponds to position 15 of SEQ ID NO:79), leucine at position 235 (which corresponds to position 16 of SEQ ID NO:79), and glycine at position 237 (which corresponds to position 18 of SEQ ID NO:79), each may be substituted with a different amino acid, such as leucine at position 234 may be substituted with an alanine residue (L234A), leucine at 235 may be substituted with an alanine residue (L235A) or with a glutamic acid residue (L235E), and the glycine residue at position 237 may be substituted with another amino acid, for example an alanine residue (G237A).

In one embodiment, a mutein Fc polypeptide that is fused in frame to a viral polypeptide (or variant or fragment thereof) comprises one, two, three, four, five, or six mutations at positions 15-20 of SEQ ID NO:79 that correspond to positions 234-239 of a human IgG1 CH2 domain (EU numbering system) as described herein. An exemplary mutein Fc polypeptide has the amino acid sequence set forth in SEQ ID NO:77 in which substitutions corresponding to (L234A), (L235E), and (G237A) may be found at positions 13, 14, and 16 of SEQ ID NO:77.

In another embodiment, a mutein Fc polypeptide comprises a mutation of a cysteine residue in the hinge region of an Fc polypeptide. In one embodiment, the cysteine residue most proximal to the amino terminus of the hinge region of an Fc polypeptide (e.g., for example, the cysteine residue most proximal to the amino terminus of the hinge region of the Fc portion of a wildtype IgG1 immunoglobulin) is deleted or substituted with another amino acid. That is, by way of illustration, the cysteine residue at position 1 of SEQ ID NO:79 is deleted, or the cysteine residue at position 1 is substituted with another amino acid that is incapable of forming a disulfide bond, for example, with a serine residue. In another embodiment, a mutein Fc polypeptide comprises a deletion or substitution of the cysteine residue most proximal to the amino terminus of the hinge region of an Fc polypeptide further comprises deletion or substitution of the adjacent C-terminal amino acid. In a certain embodiment, this cysteine residue and the adjacent C-terminal residue are both deleted from the hinge region of a mutein Fc polypeptide. In a specific embodiment, the cysteine residue at position 1 of SEQ ID NO:79 and the aspartic acid at position 2 of SEQ ID NO:79 are deleted. Fc polypeptides that comprise deletion of these cysteine and aspartic acid residues in the hinge region may be efficiently expressed in a host cell, and in certain instances, may be more efficiently expressed in a cell than an Fc polypeptide that retains the wildtype cysteine and aspartate residues.

In a specific embodiment, a mutein Fc polypeptide comprises the amino acid sequence set forth in SEQ ID NO:77, which differs from the wildtype Fc polypeptide (SEQ ID NO:79) wherein the cysteine residue at position 1 of SEQ ID NO:79 is deleted and the aspartic acid at position 2 of SEQ ID NO:79 is deleted and the leucine reside at position 15, corresponding to position EU234, of SEQ ID NO:79 is substituted with an alanine residue, the leucine residue at position 16 (which corresponds to EU235) is substituted with a glutamic acid residue, and the glycine at position 18, corresponding to EU237, is substituted with an alanine residue (see also FIG. 5). Thus, an exemplary mutein Fc polypeptide comprises an amino acid sequence at its amino terminal portion of KTHTCPPCPAPEAEGAPS (SEQ ID NO:81) (see SEQ ID NO:77, an exemplary Fc mutein sequence).

Other Fc variants encompass similar amino acid sequences of known Fc polypeptide sequences that have only minor changes, for example by way of illustration and not limitation, covalent chemical modifications, insertions, deletions and/or substitutions, which may further include conservative substitutions. Amino acid sequences that are similar to one another may share substantial regions of sequence homology. Similarly, nucleotide sequences that encode the Fc variants may encompass substantially similar nucleotide sequences and have only minor changes, for example by way of illustration and not limitation, covalent chemical modifications, insertions, deletions, and/or substitutions, which may further include silent mutations owing to degeneracy of the genetic code. Nucleotide sequences that are similar to one another may share substantial regions of sequence homology.

An Fc polypeptide or at least one immunogloblulin constant region, or portion thereof, when fused to a peptide or polypeptide of interest acts, at least in part, as a vehicle or carrier moiety that prevents degradation and/or increases half-life, reduces toxicity, reduces immunogenicity, and/or increases biological activity of the peptide such as by forming dimers or other multimers (see, e.g., U.S. Pat. Nos. 6,018,026; 6,291,646; 6,323,323; 6,300,099; 5,843,725). (See also, e.g., U.S. Pat. No. 5,428,130; U.S. Pat. No. 6,660,843; U.S. Patent Application Publication Nos. 2003/064480; 2001/053539; 2004/087778; 2004/077022; 2004/071712; 2004/057953/ 2004/053845/ 2004/044188; 2004/001853; 2004/082039). Alternative moieties to an immunoglobulin constant region such as an Fc polypeptide that may be linked or fused to a peptide that alters the immunoresponsiveness of an immune cell include, for example, a linear polymer (e.g., polyethylene glycol, polylysine, dextran, etc.; see, for example, U.S. Pat. No. 4,289,872; International Patent Application Publication No. WO 93/21259); a lipid; a cholesterol group (such as a steroid); a carbohydrate or oligosaccharide.

The nucleotide sequences that encode Fc polypeptides from various classes and isotypes of immunoglobulins from various species are known and available in GenBank databases and in Kabat (Kabat et al., in Sequences of Proteins of Immunological Interest, 4th ed., (U.S. Dept. of Health and Human Services, U.S. Government Printing Office, 1991), see also updates to the online Kabat database), any sequence of which may be used for preparing a recombinant construct according to molecular biology methods routinely practiced by persons skilled in the art. To minimize the immunogenicity of the Fc polypeptide in the host or subject to which a RPTP fragment fusion polypeptide may be administered, the sequence of the Fc polypeptide is typically chosen from immunoglobulins of the same species, that is, for example, a human Fc polypeptide sequence is fused to a RPTP fragment that will be administered to a human subject or host.

Methods that are described herein for identifying cell surface molecules such as the RPTPS that interact with and/or bind to poxvirus polypeptides such as A41L or 130L, may also be used to identify intracellular molecules that interact with, are ligands for, form a complex with, or are otherwise associated with the RPTPs described herein (i.e., LAR, RPTP-δ, and/or RPTP-σ). Without wishing to be bound by theory, identification of intracellular molecules that interact with one or more of LAR, RPTP-δ, and RPTP-σ by virtue of the interaction between a poxvirus polypeptide and the RPTP may identify particular pathways (and components thereof) involved in, or that when disrupted or activated result in, manifestation of a disease or disorder. Such intracellular molecules (for example, plakoglobulin and liprin-1-β that interact with at least LAR identified by TAP-TAG procedures using A41L) that associate with one or more of the RPTPs and that are involved with one or more signal transduction pathways may be targets for agents and compositions that are useful for treating an immunological disease or disorder, cardiovascular disease or disorder, or metabolic disease or disorder as described herein. Alternatively, agents described herein that interact with one or more of LAR, RPTP-δ, and RPTP-σ and that are useful for treating a disease or disorder and/or altering immunoresponsiveness of an immune cell may affect the interaction between the RPTP and the intracellular molecule, and thus may alter one or more biological activities of the cell.

### Agents

Binding of a poxvirus polypeptide, such as A41L or 130L, to LAR, RPTP-δ, and/or RPTP-σ alters at least one biological function of these phosphatases, and as described herein the interaction between A41L or 130L with LAR, RPTP-δ, and/or RPTP-σ expressed on the cell surface of an immune cell may alter (e.g., suppresses or enhances) the immunoresponsiveness of the cell. Alteration of the immunoresponsiveness of an immune cell may also be effected by a bioactive agent (compound or molecule) in a manner similar to a poxvirus polypeptide. Bioactive agents include, for example, small molecules, nucleic acids (such as aptamers, siRNAs, antisense nucleic acids), antibodies and fragments thereof, and fusion proteins (such as peptide-Fc fusion proteins and RPTP Ig region-Fc fusion proteins). An agent may interact with and bind to at least one of LAR, RPTP-δ, and RPTP-σ at a location on the RPTP that is the same location or proximal to the same location as where A41L or 130L binds. Alternatively, alteration of immunoresponsiveness by an agent in a manner similar to the effect of A41L (or 130L) may result from binding or interaction of the agent with the RPTP at a location distal from that at which the poxvirus polypeptide binds. Binding studies, including competitive binding assays, and functional assays, which indicate the level of immunoresponsiveness of a cell, may be performed according to methods described herein and practiced in the art to determine and compare the capability and level with which an agent binds to and affects the immunoresponsiveness of an immune cell.

Methods are provided herein for identifying an agent that alters (e.g., suppresses or enhances in a statistically or biologically significant manner) immunoresponsiveness of an immune cell and for characterizing and determining the level of suppression or enhancement of such an agent once identified. Such methods, which are discussed in greater detail herein and are familiar to persons skilled in the art, which include but are not limited to, binding assays, such as immunoassays (e.g., ELISA, radioimmunoassay, immunoblot, etc.), competitive binding assays, and surface plasmon resonance. These methods comprise contacting (mixing, combining with, or in some manner permitting interaction) among a (1) candidate agent; (2) an immune cell that expresses at least one of LAR, RPTP-σ, and RPTP-δ; and (3) a poxvirus polypeptide, such as A41L or 130L, under conditions and for a time sufficient to permit interaction between the at least one RPTP polypeptide and the poxvirus polypeptide. Conditions for a particular assay include temperature, buffers (including salts, cations, media), and other components that maintain the integrity of the cell, the agent, and the poxvirus polypeptide with which a person skilled in the art will be familiar and/or which can be readily determined. The interaction or level of binding of A41L (or 130L) to the immune cell in the presence of the candidate agent can be readily determined and compared with the level of binding of A41L (or 130L) to the cell in the absence of the agent. A decrease in the level of binding of A41L (or 130L) to the immune cell in the presence of the candidate agent indicates that the candidate agent suppresses immunoresponsiveness of the immune cell.

In another embodiment, a method for identifying an agent that alters (suppresses or enhances) immunoresponsiveness of an immune cell comprises determining the level of immunoresponsiveness of an immune cell that expresses at least one of LAR, RPTP-σ, and RPTP-δ in the presence of the agent. In certain specific embodiments, an agent is identified that suppresses immunoresponsiveness of an immune cell. Immunoresponsiveness may be determined according to methods practiced in the art such as measuring levels of cytokines, proliferation, and stimulation. Immunoresponsiveness of an immune cell may also be determined by evaluating changes in cell adhesion and cell migration and by examining the tyrosine phosphorylation pattern of cellular proteins, including but not limited to cytoskeletal proteins and other proteins that affect cell adhesion and migration.

Numerous assays and techniques are practiced by persons skilled in the art for determining the interaction between or binding between a biological molecule and a cognate ligand. Accordingly, interaction between a poxvirus polypeptide such as A41L or 130L and any one or more of LAR, RPTP-σ, and RPTP-δ, including the effect of a bioactive agent on this interaction and/or binding in the presence of the agent, can be readily determined by such assays and techniques as described in detail herein.

### Small Molecules

Bioactive agents may also include natural and synthetic molecules, for example, small molecules that bind to a poxvirus polypeptide (e.g., A41L or 130L), or to one or more of LAR, RPTP-σ, and RPTP-δ, and/or to a complex between the poxvirus polypeptide (e.g., A41L or 130L) and any one of LAR, RPTP-σ, and RPTP-δ. Candidate agents for use in a method of screening for an agent that alters (suppresses or enhances) immunoresponsiveness of an immune cell and/or that inhibits binding of the poxvirus polypeptide (e.g., A41L or 130L) to at least one, at least two, or all three of LAR, RPTP-σ, and RPTP-δ, may be provided as “libraries” or collections of compounds, compositions, or molecules.

Such molecules typically include compounds known in the art as “small molecules” and have molecular weights less than 10⁵ daltons, less than 10⁴ daltons, or less than 10³ daltons. For example, members of a library of test compounds can be administered to a plurality of samples, each containing at least one tyrosine phosphatase polypeptide as provided herein, and then the samples are assayed for their capability to enhance or inhibit LAR, RPTP-σ, and/or RPTP-δ-mediated dephosphorylation of, or binding to, a substrate, the capability to inhibit or enhance binding of the phosphatase to the poxvirus polypeptide (e.g., A41L or 130L); and/or the capability of the test compounds to modulate immunoresponsiveness of immune cells. Compounds so identified as capable of affecting at least one function of the poxvirus polypeptide LAR, RPTP-σ, and/or RPTP-δ are valuable for therapeutic and/or diagnostic purposes, since they permit treatment and/or detection of diseases associated with LAR, RPTP-σ, and/or RPTP-δ activity. Such compounds are also valuable in research directed to molecular signaling mechanisms that involve any one or more of LAR, RPTP-σ, and/or RPTP-δ.

Candidate agents further may be provided as members of a combinatorial library, which preferably includes synthetic agents prepared according to a plurality of predetermined chemical reactions performed in a plurality of reaction vessels. For example, various starting compounds may be prepared according to one or more of solid-phase synthesis, recorded random mix methodologies, and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., International Patent Application Nos. PCT/US91/08694 and PCT/US91/04666) or other compositions that may include small molecules as provided herein (see, e.g., International Patent Application No. PCT/US94/08542, EP Patent No. 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures and tested according to the present disclosure.

Examples of methods for the synthesis of molecular libraries can be found in the art, for example in DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and in Gallop et al., J. Med. Chem. 37:1233 (1994). Libraries of compounds may be presented in solution (e.g., Houghten, Biotechniques 13:412-21 (1992)); or on beads (Lam, Nature 354:82-84 (1991)); chips (Fodor, Nature 364:555-56 (1993)); bacteria (Ladner, U.S. Pat. No. 5,223,409); spores (Ladner, supra); plasmids (Cull et al., Proc. Natl. Acad. Sci. USA 89:1865-69 (1992)); or on phage (Scott and Smith, Science 249:386-390 (1990); Devlin, Science 249:404-406 (1990); Cwirla et al., Proc. Natl. Acad. Sci. USA 87:6378-82 (1990); Felici, J. Mol. Biol. 222:301-10 (1991); Ladner, supra).

### Peptide-Immunoglobulin Constant Region Fusion Polypeptides

In one embodiment, a bioactive agent that is used for altering the immunoresponsiveness of an immune cell and that may be used for treating an immunological disease or disorder is a peptide-immunoglobulin (Ig) constant region fusion polypeptide, which includes a peptide-IgFc fusion polypeptide. The peptide may be any naturally occurring or recombinantly prepared molecule. A peptide-Ig constant region fusion polypeptide, such as a peptide-IgFc fusion polypeptide (also referred to in the art as a peptibody (see, e.g., U.S. Pat. No. 6,660,843)), comprises a biologically active peptide or polypeptide capable of altering the activity of a protein of interest, such as an RPTP ((LAR, RPTP-σ, and/or RPTP-δ) expressed by an immune cell, that is fused with a portion, at least one constant region domain (e.g., CH1, CH2, CH3, and/or CH4), or the Fc polypeptide (CH2-CH3) of an immunoglobulin. The Fc polypeptide is also referred to herein as the Fc portion or the Fc region.

In one embodiment, the peptide portion of the fusion polypeptide is capable of interacting with or binding to at least one of, at least two of, or all three of LAR, RPTP-σ, and RPTP-δ, and effecting the same biological activity as a poxvirus polypeptide (e.g., A41L or 130L) when it binds to at least one of the RPTPs, thus suppressing (inhibiting, preventing, decreasing, or abrogating) the immunoresponsiveness of the immune cell expressing the RPTP. Methods are provided herein for identifying a peptide that is capable of altering (e.g., suppressing) immunoresponsiveness of an immune cell (that is, a peptide that acts as an A41L or 130L mimic). For example, such a peptide may be identified by determining its capability to inhibit or block binding of A41L (or 130L) to a cell that expresses at least one of the RPTPs. Alternatively, a candidate peptide may be permitted to contact or interact with an immune cell that expresses at least one of the RPTPs, and the capability of the candidate peptide to suppress or enhance immunoresponsiveness of the immune cell can be measured according to methods described herein and practiced in the art. Candidate peptides may be provided as members of a combinatorial library, which includes synthetic peptides prepared according to a plurality of predetermined chemical reactions performed in a plurality of reaction vessels. For example, various starting peptides may be prepared according to standard peptide synthesis techniques with which a skilled artisan will be familiar.

Peptides that alter the immunoresponsiveness of an immune cell may be identified and isolated from combinatorial libraries (see, e.g., International Patent Application Nos. PCT/US91/08694 and PCT/US91/04666) and from phage display peptide libraries (see, e.g., Scott et al., Science 249:386 (1990); Devlin et al., Science 249:404 (1990); Cwirla et al., Science 276: 1696-99 (1997); U.S. Pat. No. 5,223,409; U.S. Pat. No. 5,733,731; U.S. Pat. No. 5,498,530; U.S. Pat. No. 5,432,018; U.S. Pat. No. 5,338,665; 1994; U.S. Pat. No. 5,922,545; International Application Publication Nos. WO 96/40987 and WO 98/15833). In phage display peptide libraries, random peptide sequences are fused to a phage coat protein such that the peptides are displayed on the external surface of a filamentous phage particle. Typically, the displayed peptides are contacted with a ligand or binding molecule of interest to permit interaction between the peptide and the ligand or binding molecule, unbound phage are removed, and the bound phage are eluted and subsequently enriched by successive rounds of affinity purification and repropagation. The peptides with the greatest affinity for the ligand or binding molecule or target molecule of interest (e.g., the RPTPs described herein) may be sequenced to identify key residues, which may identify peptides within one or more structurally related families of peptides. Comparison of sequences of peptides may also indicate which residues in such peptides may be safely substituted or deleted by mutagenesis. These peptides may then be incorporated into additional peptide libraries that can be screened and peptides with optimized affinity can be identified.

Additional methods for identifying peptides that may alter the immunoresponsiveness of an immune cell and thus be useful for treating and/or preventing an immunological disease or disorder include, but are not limited to, (1) structural analysis of protein-protein interaction such as analyzing the crystal structure of the RPTP target (see, e.g., Jia, Biochem. Cell Biol. 75:17-26 (1997)) to identity and to determine the orientation of critical residues of the RPTP, which will be useful for designing a peptide (see, e.g., Takasaki et al., Nature Biotech. 15: 1266-70 (1997)); (2) a peptide library comprising peptides fused to a peptidoglycan-associated lipoprotein and displayed on the outer surface of bacteria such as E. coli; (3) generating a library of peptides by disrupting translation of polypeptides to generate RNA-associated peptides; and (4) generating peptides by digesting polypeptides with one or more proteases. (See also, e.g., U.S. Pat. Nos. 6,660,843; 5,773,569; 5,869,451; 5,932,946; 5,608,035; 5,786,331; 5,880,096). A peptide may comprise any number of amino acids between 3 and 75 amino acids, 3 and 60 amino acids, 3 and 50 amino acids, 3 and 40 amino acids, 3 and 30 amino acids, 3 and 20 amino acids, or 3 and 10 amino acids. A peptide that has the capability of alter the immunoresponsiveness of an immune cell (e.g., in certain embodiments, to suppress the immunoresponsiveness of the immune cell and in certain other embodiments, to enhance immunoresponsiveness of the immune cell) may also be further derivatized to add or insert amino acids that are useful for constructing a peptide-Ig constant region fusion protein (such as amino acids that are linking sequences or that are spacer sequences).

A peptide that may be used to construct a peptide-Ig constant region fusion polypeptide (including a peptide-IgFc fusion polypeptide) may be derived from a poxvirus polypeptide, such as an A41L polypeptide or 130L polypeptide. A41L or 130L peptides may be randomly generated by proteolytic digestion using any one or more of various proteases, isolated, and then analyzed for their capability to alter the immunoresponsiveness of an immune cell. Such peptides may also be generated using recombinant methods described herein and practiced in the art. Randomly generated peptides may also be used to prepare peptide combinatorial libraries or phage libraries as described herein and in the art. Alternatively, the amino acid sequences of portions of A41L or 130L that interact with LAR, RPTP-σ, and/or RPTP-δ may be determined by computer modeling of the phosphatase, or of a portion of the phosphatase, for example, the extracellular portion or the Ig domains, and/or x-ray crystallography (which may include preparation and analysis of crystals of the phosphatase only or of the phosphatase-viral polypeptide complex).

As described in detail above, an Fc polypeptide of an immunoglobulin comprises the heavy chain CH2 domain and CH3 domain and a portion of or the entire hinge region that is located between CH1 and CH2. Fc regions are monomeric polypeptides that may be linked into dimeric or multimeric forms by covalent (e.g., particularly disulfide bonds) and non-covalent association. The number of intermolecular disulfide bonds between monomeric subunits of Fc polypeptides varies depending on the immunoglobulin class (e.g., IgG, IgA, IgE) or subclass (e.g., human IgG1, IgG2, IgG3, IgG4, IgA1, IgA2). Presently, an Fc polypeptide, and any one or more constant region domains, and fusion proteins comprising at least one immunoglobulin (Ig) constant region domain can be readily prepared according to recombinant molecular biology techniques with which a skilled artisan is quite familiar.

The Fc polypeptide is preferably prepared using the nucleotide and the encoded amino acid sequences derived from the animal species for whose use the peptide-IgFc fusion polypeptide is intended. In one embodiment, the Fc polypeptide is of human origin and may be from any of the immunoglobulin classes, such as human IgG1 and IgG2.

An Fc polypeptide as described herein also includes Fc polypeptide variants. One such Fc polypeptide variant has one or more cysteine residues (such as one or more cysteine residues in the hinge region) that forms a disulfide bond with another Fc polypeptide substituted with another amino acid, such as serine, to reduce the number of disulfide bonds formed between two Fc polypeptides. Alternatively, one or more cysteine residues may be deleted from the wildtype hinge of the Fc polypeptide.

Another example of an Fc polypeptide variant is a variant that has one or more amino acids involved in an effector function substituted or deleted such that the Fc polypeptide has a reduced level of an effector function. For example, amino acids in the Fc region may be substituted to reduce or abrogate binding of a component of the complement cascade (see, e.g., Duncan et al., Nature 332:563-64 (1988); Morgan et al., Immunology 86:319-24 (1995)) or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity. Such an Fc polypeptide variant that differs from the wildtype Fc polypeptide is also called herein a mutein Fc polypeptide.

In one embodiment, a peptide as described herein is fused in frame with an Fc polypeptide that comprises at least one substitution of a residue that in the wildtype Fc region polypeptide contributes to binding of an Fc polypeptide or immunoglobulin to one or more IgG Fc receptors expressed on certain immune cells. Such a mutein Fc polypeptide comprises at least one substitution of an amino acid residue in the CH2 domain of the mutein Fc polypeptide, such that the capability of the fusion polypeptide to bind to an IgG Fc receptor, such as an IgG Fc receptor present on the surface of an immune cell, is reduced. The types of Fc IgG receptors expressed on human leukocytes are described in detail above.

Residues in the amino terminal portion of the CH2 domain that contribute to IgG Fc receptor binding include residues at positions Leu234-Ser239 (Leu-Leu-Gly-Gly-Pro-Ser (SEQ ID NO:80) (EU numbering system, Kabat et al., supra) (see, e.g., Morgan et al., Immunology 86:319-24 (1995), and references cited therein). These positions correspond to positions 15-20 of the amino acid sequence of a human IgG1 Fc polypeptide (SEQ ID NO:79). Substitution of the amino acid at one or more of these six positions (i.e., one, two, three, four, five, or all six) in the CH2 domain results in a reduction of the capability of the Fc polypeptide to bind to one or more of the IgG Fc receptors (or isoforms thereof) (see, e.g., Burton et al., Adv. Immunol. 51:1 (1992); Hulett et al., Adv. Immunol. 57:1 (1994); Jefferis et al., Immunol. Rev. 163:59 (1998); Lund et al., J. Immunol. 147:2657 (1991); Sarmay et al., Mol. Immunol. 29:633 (1992); Lund et al., Mol. Immunol. 29:53 (1992); Morgan et al., supra). In addition to substitution of one or more amino acids at EU positions 234-239, one, two, or three or more amino acids adjacent to this region (either to the carboxy terminal side of position 239 or to the amino terminal side of position 234) may also be substituted.

By way of example, substitution of the leucine residue at position 235 (which corresponds to position 16 of SEQ ID NO:79) with a glutamic acid residue or an alanine residue abolishes or reduces, respectively, the affinity of an immunoglobulin (such as human IgG3) for FcγRI (Lund et al., 1991, supra; Canfield et al., supra; Morgan et al., supra). As another example, replacement of the leucine residues at positions 234 and 235 (which correspond to positions 15 and 16 of SEQ ID NO:79), for example, with alanine residues, abrogates binding of an immunoglobulin to FcγRIIa (see, e.g., Wines et al., supra). Alternatively, leucine at position 234 (which corresponds to position 15 of SEQ ID NO:79), leucine at position 235 (which corresponds to position 16 of SEQ ID NO:79), and glycine at position 237 (which corresponds to position 18 of SEQ ID NO:79), each may be substituted with a different amino acid, such as leucine at position 234 may be substituted with an alanine residue (L234A), leucine at 235 may be substituted with an alanine residue (L235A) or with a glutamic acid residue (L235E), and the glycine residue at position 237 may be substituted with another amino acid, for example an alanine residue (G237A).

In one embodiment, a mutein Fc polypeptide that is fused in frame to a viral polypeptide (or variant or fragment thereof) comprises one, two, three, four, five, or six mutations at positions 15-20 of SEQ ID NO:79 that correspond to positions 234-239 of a human IgG1 CH2 domain (EU numbering system) as described herein. An exemplary mutein Fc polypeptide has the amino acid sequence set forth in SEQ ID NO:77 in which substitutions corresponding to (L234A), (L235E), and (G237A) may be found at positions 13, 14, and 16 of SEQ ID NO:77.

In another embodiment, a mutein Fc polypeptide comprises a mutation of a cysteine residue in the hinge region of an Fc polypeptide. In one embodiment, the cysteine residue most proximal to the amino terminus of the hinge region of an Fc polypeptide (e.g., for example, the cysteine residue most proximal to the amino terminus of the hinge region of the Fc portion of a wildtype IgG1 immunoglobulin) is deleted or substituted with another amino acid. That is, by way of illustration, the cysteine residue at position 1 of SEQ ID NO:79 is deleted, or the cysteine residue at position 1 is substituted with another amino acid that is incapable of forming a disulfide bond, for example, with a serine residue. In another embodiment, a mutein Fc polypeptide comprises a deletion or substitution of the cysteine residue most proximal to the amino terminus of the hinge region of an Fc polypeptide further comprises deletion or substitution of the adjacent C-terminal amino acid. In a certain embodiment, this cysteine residue and the adjacent C-terminal residue are both deleted from the hinge region of a mutein Fc polypeptide. In a specific embodiment, the cysteine residue at position 1 of SEQ ID NO:79 and the aspartic acid at position 2 of SEQ ID NO:79 are deleted. Fc polypeptides that comprise deletion of these cysteine and aspartic acid residues in the hinge region may be efficiently expressed in a host cell, and in certain instances, may be more efficiently expressed in a cell than an Fc polypeptide that retains the wildtype cysteine and aspartate residues.

In a specific embodiment, a mutein Fc polypeptide comprises the amino acid sequence set forth in SEQ ID NO:77, which differs from the wildtype Fc polypeptide (SEQ ID NO:79) wherein the cysteine residue at position 1 of SEQ ID NO:79 is deleted and the aspartic acid at position 2 of SEQ ID NO:79 is deleted and the leucine reside at position 15 of SEQ ID NO:79 is substituted with an alanine residue, the leucine residue at position 16 is substituted with a glutamic acid residue, and the glycine at position 18 is substituted with an alanine residue (see also FIG. 5). Thus, an exemplary mutein Fc polypeptide comprises an amino acid sequence at its amino terminal portion of KTHTCPPCPAPEAEGAPS (SEQ ID NO:81) (see SEQ ID NO:77, an exemplary Fc mutein sequence).

Other Fc variants encompass similar amino acid sequences of known Fc polypeptide sequences that have only minor changes, for example by way of illustration and not limitation, covalent chemical modifications, insertions, deletions and/or substitutions, which may further include conservative substitutions. Amino acid sequences that are similar to one another may share substantial regions of sequence homology. Similarly, nucleotide sequences that encode the Fc variants may encompass substantially similar nucleotide sequences and have only minor changes, for example by way of illustration and not limitation, covalent chemical modifications, insertions, deletions, and/or substitutions, which may further include silent mutations owing to degeneracy of the genetic code. Nucleotide sequences that are similar to one another may share substantial regions of sequence homology.

An Fc polypeptide or at least one immunogloblulin constant region, or portion thereof, when fused to a peptide or polypeptide of interest acts, at least in part, as a vehicle or carrier moiety that prevents degradation and/or increases half-life, reduces toxicity, reduces immunogenicity, and/or increases biological activity of the peptide such as by forming dimers or other multimers (see, e.g., U.S. Pat. Nos. 6,018,026; 6,291,646; 6,323,323; 6,300,099; 5,843,725). (See also, e.g., U.S. Pat. No. 5,428,130; U.S. Pat. No. 6,660,843; U.S. Patent Application Publication Nos. 2003/064480; 2001/053539; 2004/087778; 2004/077022; 2004/071712; 2004/057953/ 2004/053845/ 2004/044188; 2004/001853; 2004/082039). Alternative moieties to an immunoglobulin constant region such as an Fc polypeptide that may be linked or fused to a peptide that alters the immunoresponsiveness of an immune cell include, for example, a linear polymer (e.g., polyethylene glycol, polylysine, dextran, etc.; see, for example, U.S. Pat. No. 4,289,872; International Patent Application Publication No. WO 93/21259); a lipid; a cholesterol group (such as a steroid); a carbohydrate or oligosaccharide.

### Nucleic Acid Molecules

In certain embodiments, polynucleotides and oligonucleotides are provided that are complementary to at least a portion of a sequence encoding an RPTP (LAR, RPTP-σ, or RPTP-δ) (e.g., a short interfering nucleic acid, an antisense polynucleotide, a ribozyme, or a peptide nucleic acid) and that may be used to alter gene and/or protein expression. As described herein, these polynucleotides that specifically bind to or hybridize to nucleic acid molecules that encode an RPTP (LAR, RPTP-σ, or RPTP-δ) may be prepared using the nucleotide sequences provided herein and available in the art (e.g., SEQ ID NOS:23 and 27 that encode LAR; SEQ ID NOS:30, 32, 34, 36 that encode RPTP-σ; and SEQ ID NOS:38, 40, 42, 44 that encode RPTP-δ). In another embodiment, nucleic acid molecules such as aptamers that are not sequence-specific may also be used to alter gene and/or protein expression.

RNA Interference (RNAi)

By way of background, RNA interference refers to the process of sequence-specific post-transcriptional gene silencing in animals mediated by short interfering RNAs (siRNAs) (Zamore et al., Cell, 101:25-33 (2000); Fire et al., Nature 391:806 (1998); Hamilton et al., Science 286:950-51 (1999); Lin et al., Nature 402:128-29 (1999); Sharp, Genes & Dev. 13:139-41 (1999); and Strauss, Science 286:886 (1999); Sandy et al., Biotechniques 39:215-24 (2005)); U.S. Pat. Nos. 6,506,559; 6,573,099; International Patent Application Publication No. WO 01/75164). Inhibition is sequence-specific in that a nucleotide sequence from a portion of the target gene (for example, a gene expressing an RPTP described herein) is chosen to produce inhibitory RNA. The process of post-transcriptional gene silencing is thought to be a cellular defense mechanism used to prevent the expression of foreign genes (Fire et al., Trends Genet. 15:358 (1999)). The process comprises introducing into the cell a nucleic acid molecule, generally, RNA, with partial or fully double-stranded character. The presence of dsRNA in cells triggers the RNAi response through a mechanism that has yet to be fully characterized. This mechanism appears to be different from other mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see, e.g., U.S. Pat. Nos. 6,107,094; 5,898,031; Clemens et al., J. Interferon Cytokine Res. 17:503-24 (1997); Adah et al., Curr. Med. Chem. 8:1189 (2001)).

The presence of long dsRNAs in cells stimulates the activity of a ribonuclease III enzyme referred to as dicer (Bass, Cell 101:235 (2000); Zamore et al., Cell, 101:25-33 (2000); Hammond et al., Nature 404:293 (2000)). Dicer is involved in the processing of the dsRNA into the short pieces of dsRNA known as siRNAs (Zamore et al., Cell 101:25-33 (2000); Bass, Cell 101:235 (2000); Berstein et al., Nature 409:363 (2001)). Short interfering RNAs derived from dicer activity are typically about 21 to about 23 nucleotides in length and comprise about 19 base pair duplexes (e.g., a 21-22 nucleotide long dsRNA molecule that contains a 19-base pair duplex core and two unpaired nucleotides at each 3′ end) (Zamore et al., 2000, supra; Elbashir et al., 2001, supra; Dykxhoorn et al., Nat. Rev. Mol. Cell Biol. 4:457-67 (2003)). Dicer has also been implicated in the excision of 21- and 22-nucleotide small temporal RNAs (stRNAs) from precursor RNA of conserved structure that are implicated in translational control (Hutvagner et al., Science 293:834 (2001)). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA occurs in the middle of the region complementary to the antisense strand of the siRNA duplex (Elbashir et al., 2001, supra).

Short interfering RNAs may be used for modulating (decreasing or inhibiting) the expression of LAR, RPTP-σ, and/or RPTP-δ genes. The disclosure herein relates to compounds, compositions, and methods useful for modulating the expression and activity of genes that encode the RPTPs, LAR, RPTP-σ, and RPTP-δ, by RNA interference using small nucleic acid molecules. In particular, small nucleic acid molecules, such as short interfering RNA (siRNA), micro-RNA (miRNA), and short hairpin RNA (shRNA) molecules may be used according to the methods described herein to modulate the expression of LAR, RPTP-σ, and/or RPTP-δ, or variants thereof. A siRNA polynucleotide preferably comprises a double-stranded RNA (dsRNA) but may comprise a single-stranded RNA (see, e.g., Martinez et al. Cell 110:563-74 (2002)). A siRNA polynucleotide may comprise other naturally occurring, recombinant, or synthetic single-stranded or double-stranded polymers of nucleotides (ribonucleotides or deoxyribonucleotides or a combination of both) and/or nucleotide analogues as provided herein and known and used by persons skilled in the art.

At least one strand of a double-stranded siRNA polynucleotide has at least one, and preferably two nucleotides that “overhang” (i.e., that do not base pair with a complementary base in the opposing strand) at the 3′ end of either strand, or preferably both strands, of the siRNA polynucleotide. Typically, each strand of the siRNA polynucleotide duplex has a two-nucleotide overhang at the 3′ end. The two-nucleotide overhang may be a thymidine dinucleotide (TT) or may comprise other bases, for example, a TC dinucleotide or a TG dinucleotide, or any other dinucleotide (see, e.g., International Patent Application Publication No. WO 01/75164). Alternatively, the siRNA polynucleotide may have blunt ends, that is, each nucleotide in one strand of the duplex is perfectly complementary (e.g., by Watson-Crick base-pairing) with a nucleotide of the opposite strand.

A siRNA may be transcribed using as a template a DNA (genomic, cDNA, or synthetic) that contains a RNA polymerase promoter, for example, a U6 promoter or the H1 RNA polymerase III promoter, or the siRNA may be a synthetically derived RNA molecule. The double-stranded structure of an siRNA may be formed by a single self-complementary RNA strand or from two complementary RNA strands. RNA duplex formation may be initiated either inside or outside the cell. The RNA may be introduced in an amount to deliver at least one copy per cell or at least 5, 10, 50, 100, 250, 500, or 1000 copies per cell. Polynucleotides that are siRNA polynucleotides may be derived from a single-stranded polynucleotide that comprises a single-stranded oligonucleotide fragment (e.g., of about 15-30 nucleotides, of about 19-25 nucleotides, or of about 19-22 nucleotides, which should be understood to include any whole integer of nucleotides including and between 15 and 30) and its reverse complement, typically separated by a spacer sequence. According to certain such embodiments, cleavage of the spacer provides the single-stranded oligonucleotide fragment and its reverse complement, such that they may anneal to form the double-stranded siRNA polynucleotide. Optionally, additional processing steps may result in addition or removal of one, two, three or more nucleotides from the 3′ end and/or the 5′ end of either or both strands. In certain embodiments the spacer is of a length that permits the fragment and its reverse complement to anneal and form a double-stranded structure (e.g., like a hairpin polynucleotide) prior to cleavage of the spacer (and, optionally, subsequent processing steps that may result in addition or removal of one, two, three, four, or more nucleotides from the 3′ end and/or the 5′ end of either or both strands). A spacer sequence may therefore be any polynucleotide sequence that is situated between two complementary polynucleotide sequence regions which, when annealed into a double-stranded nucleic acid, comprise a siRNA polynucleotide. A spacer sequence may comprise at least 4 nucleotides, although in certain embodiments the spacer may comprise 5, 6, 7, 8, 9, 10, 11, 12, 13, 14, 15, 16, 17, 18, 19, 20, 21-25, 26-30, 31-40, 41-50, 51-70, 71-90, 91-110, 111-150, 151-200 or more nucleotides. Examples of siRNA polynucleotides derived from a single nucleotide strand comprising two complementary nucleotide sequences separated by a spacer have been described (e.g., Brummelkamp et al., 2002 Science 296:550; Paddison et al., 2002 Genes Develop. 16:948; Paul et al. Nat. Biotechnol. 20:505-508 (2002); Grabarek et al., Biotechniques 34:734-44 (2003)).

A vector suitable for expression of an siRNA polynucleotide may comprise a recombinant nucleic acid construct containing one or more promoters for transcription of an RNA molecule, for example, the human U6 snRNA promoter (see, e.g., Miyagishi et al, Nat. Biotechnol. 20:497-500 (2002); Lee et al., Nat. Biotechnol. 20:500-505 (2002); Paul et al., Nat. Biotechnol. 20:505-508 (2002); Grabarek et al., BioTechniques 34:73544 (2003); see also Sui et al., Proc. Natl. Acad. Sci. USA 99:5515-20 (2002)). Each strand of a siRNA polynucleotide may be transcribed separately, each under the direction of a separate promoter, and then may hybridize within the cell to form the siRNA polynucleotide duplex. Each strand may also be transcribed from separate vectors (see Lee et al., supra). Alternatively, the sense and antisense sequences specific for a RPTP (LAR, RPTP-σ, and/or RPTP-δ) sequence may be transcribed under the control of a single promoter such that the siRNA polynucleotide forms a hairpin molecule (Paul et al., supra). In this instance, the complementary strands of the siRNA specific sequences are separated by a spacer that comprises at least four nucleotides, but may comprise at least 5, 6, 7, 8, 9, 10, 11, 12, 13, 14, 15, 16, 17, or 18 nucleotides or more nucleotides as described herein. In addition, siRNAs transcribed under the control of a U6 promoter that form a hairpin may have a stretch of about four uridines at the 3′ end that act as the transcription termination signal (Miyagishi et al., supra; Paul et al., supra). By way of illustration, if the target sequence is 19 nucleotides, the siRNA hairpin polynucleotide (beginning at the 5′ end) has a 19-nucleotide sense sequence followed by a spacer (which has two uridine nucleotides adjacent to the 3′ end of the 19-nucleotide sense sequence), and the spacer is linked to a 19 nucleotide antisense sequence followed by a 4-uridine terminator sequence, which results in an overhang. Short interfering RNA polynucleotides with such overhangs effectively interfere with expression of the target polypeptide (see Miyagishi et al., supra; Paul et al., supra). A recombinant construct may also be prepared using another RNA polymerase III promoter, the H1 RNA promoter, that may be operatively linked to siRNA polynucleotide specific sequences, which may be used for transcription of hairpin structures comprising the siRNA specific sequences or separate transcription of each strand of a siRNA duplex polynucleotide (see, e.g., Brummelkamp et al., Science 296:550-53 (2002); Paddison et al., supra). DNA vectors useful for insertion of sequences for transcription of an siRNA polynucleotide include pSUPER vector (see, e.g., Brummelkamp et al., supra); pAV vectors derived from pCWRSVN (see, e.g., Paul et al., supra); and pIND (see, e.g., Lee et al., supra), or the like.

RPTP polypeptides can be expressed in mammalian cells, yeast, bacteria, or other cells under the control of appropriate promoters, thus systems are provided and available for identifying and characterizing siRNA polynucleotides that are capable of interfering with polypeptide expression as provided herein. Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

These siRNAs may be used for inhibiting, decreasing, or abrogating expression of one or more of LAR, RPTP-σ, and RPTP-δ, or variants thereof, thus altering the immunoresponsiveness of an immune cell, and may be used for treating a subject or host who has an inflammatory or autoimmune disease, or a cardiovascular or metabolic disease related to expression or overexpression of one or more of the RPTPs. Interference of expression of rat LAR, RPTP-σ, and RPTP-δ in hippocampal neurons has been effective using siRNA molecules (Dunah et al., Nat. Neurosci. 8:458-67 (2005)).

In one embodiment, a siRNA molecule has RNAi activity that affects expression of LAR RNA, wherein the siRNA molecule comprises a sequence complementary to an RNA molecule that encodes an LAR polypeptide or variant thereof, including, but not limited to those sequences described herein. In another embodiment, a siRNA molecule has RNAi activity that affects expression of RPTP-σ or RPTP-δ RNA, wherein the siRNA molecule comprises a sequence complementary to an RNA that encodes a RPTP-σ or that encodes a RPTP-δ polypeptide, respectively, or variant thereof, including, but not limited to those sequences described herein. In certain other embodiments, a siRNA molecule has RNAi activity that affects expression of at least two of LAR RNA, RPTP-σ RNA, and RPTP-δ RNA. Such siRNAs that inhibit, effect a decrease, or abrogate expression of the at least two encoded RPTP(s) recognize, bind to, or hybridize to portions of the encoding sequence that are common and identical to the at least two RPTP nucleotide sequences. In another embodiment, a siRNA may inhibit, effect a decrease, or abrogate expression of LAR RNA, RPTP-σ RNA, and RPTP-δ RNA and recognize, bind to, or hybridize to portions of the encoding sequence that are common and identical to the all three RPTP nucleotide sequences.

As described herein nucleotide sequences that encode each of LAR, RPTP-σ, and RPTP-δ share sequence identity at particular locations in the polynucleotides. Such homologous or identical sequences can be identified according to methods known in the art and described herein, for example using sequence alignments. siRNA molecules can be designed to target such homologous sequences, for example using perfectly complementary sequences or by incorporating non-canonical base pairs, for example mismatches and/or wobble base pairs, that can provide additional target sequences (see, e.g., U.S. Patent Application No. 2005/0137155).

A siRNA molecule comprises an antisense strand having a nucleotide sequence that is complementary to a nucleotide sequence or a portion thereof encoding a LAR, RPTP-σ, and/or RPTP-δ polypeptide and may further comprise a sense strand, wherein the sense strand comprises a nucleotide sequence of a LAR, RPTP-σ, and/or RPTP-δ gene or mRNA, or a portion thereof. In one embodiment a siRNA molecule comprises an antisense strand having about 15, 16, 17, 18, 19, 20, or 21 nucleotides and in another embodiment about 19 to about 30 (e.g., about 19, 20, 21, 22, 23, 24, 25, 26, 27, 28, 29, or 30) nucleotides, wherein the antisense strand is complementary to a RNA sequence encoding one or more of LAR, RPTP-σ, and RPTP-δ. In certain other embodiments, the siRNA further comprises a sense strand having about 16, 17, 18, 19, 20, or 21 nucleotides and in another embodiment about 19 to about 30 (e.g., about 19, 20, 21, 22, 23, 24, 25, 26, 27, 28, 29, or 30) nucleotides. The sense strand and the antisense strand are distinct nucleotide sequences with at least about 19 complementary nucleotides. The nucleotide sequence of the siRNA polynucleotide may be identical to portion of a polynucleotide sequence that encodes an RPTP as described herein or the nucleotide sequence may differ by one, two, three, or four nucleotides. Single point mutations relative to the target sequence have been found to be effective for inhibition.

A variety of algorithms are available for determining the sequence of siRNA molecules. In general, regions of a target polynucleotide sequence to be avoided when designing an siRNA include (1) regions within 50-100 base pairs of the start codon or the termination codon; (2) intron regions; (3) stretches of 4 or more identical bases; (4) regions with GC content less than 30% or greater than 60%; and (5) repeats and low complex sequence. One algorithm that may be used for designing a siRNA that inhibits expression of a LAR, RPTP-σ, and/or RPTP-δ gene or mRNA is referred to as the Tuschl rules (Elbashir et al., Nature 411:494-98 (2001); Elbashir et al. EMBO J. 20:6877-88 (2001); Elbashir et al., Methods 26:199-213 (2002)). A target region is selected that is 50-100 nucleotides downstream of a start codon, which sequence comprises in order of preference (1) 23 nucleotides sequence motif AA(N₁₉); (2) 23 nucleotide sequence motif (NA(N₂₁); convert the 3′ end of the sense siRNA to TT; (3) NAR(N₁₇)YNN, wherein R=A or G (purine); Y−T or C (pyrimidine), and N=any nucleotide. The target sequence should have a GC content of approximately 50%. Another method referred to as rational siRNA design (Dharmacon, Inc.) assigns point values to particular sequence characteristics (see, e.g., Reynolds et al., Nat. Biotechnol. 22:326-30 (2004)). In addition, several vendors design and manufacture siRNA molecules based on the target sequence using proprietary algorithms (see, e.g., Ambion, Inc., Austin, Tex., algorithm developed by Cenix Bioscience; Qiagen, Inc., Valencia, Calif.).

A siRNA can be unmodified or chemically-modified and can be chemically synthesized, expressed from a vector, or enzymatically synthesized. The use of chemically-modified siRNA improves various properties of native siRNA molecules by, for example, increasing resistance to nuclease degradation in vivo and/or through improved cellular uptake (see, e.g., U.S. Patent Application No. 2005/0137155).

Inhibition of gene expression refers to the absence (or observable decrease) in the level of protein and/or mRNA product from a target gene encoding LAR, RPTP-σ, or RPTP-δ. Specificity refers to the ability to inhibit the target gene without manifest effects on other genes of the cell. The consequences of inhibition can be confirmed by examination of properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Examples of reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline.

Antisense Polynucleotides and Ribozymes

Antisense polynucleotides bind in a sequence-specific manner to nucleic acids such as mRNA or DNA. Identification of oligonucleotides and ribozymes for use as antisense agents and identification of DNA encoding the genes for targeted delivery involve methods well known in the art. For example, the desirable properties, lengths, and other characteristics of such oligonucleotides are well known. Antisense technology can be used to control gene expression through interference with binding of polymerases, transcription factors, or other regulatory molecules (see Gee et al., In Huber and Carr, Molecular and Immunologic Approaches, Futura Publishing Co. (Mt. Kisco, N.Y.; 1994)). An antisense polynucleotide may also alter gene expression of any one of LAR, RPTP-σ, and/or RPTP-δ by specifically hybridizing to a portion of the encoding gene or mRNA that is untranslated and may be a sequence that is a regulatory sequence. Such an antisense molecule may be designed to hybridize with a control region of an RPTP gene (e.g., promoter, enhancer or transcription initiation site) and block transcription of the gene or block translation by inhibiting binding of a transcript to ribosomes.

When bound to mRNA that has complementary sequences, antisense prevents translation of the mRNA (see, e.g., U.S. Pat. No. 5,168,053; U.S. Pat. No. 5,190,931; U.S. Pat. No. 5,135,917; U.S. Pat. No. 5,087,617; Clusel et al., Nucleic Acids Res. 21:3405-3411 (1993), which describes dumbbell antisense oligonucleotides). Triplex molecules refer to single DNA strands that bind duplex DNA forming a colinear triplex molecule, thereby preventing transcription (see, e.g., U.S. Pat. No. 5,176,996, which describes methods for making synthetic oligonucleotides that bind to target sites on duplex DNA; see also, e.g., Helene, Anticancer Drug Des. 6:569-84 (1991); Helene et al., Ann. N.Y. Acad. Sci. 660:27-36 (1992); Maher, Bioassays 14:807-15 (1992)).

An antisense polynucleotide comprises a nucleotide sequence that is complementary to a sense polynucleotide encoding a protein, for example, complementary to the coding strand of a double-stranded cDNA molecule or complementary to an mRNA sequence. Accordingly, an antisense polynucleotide can hydrogen bond to a sense polynucleotide. The antisense polynucleotide can be complementary to an entire RPTP coding strand, or to only a portion thereof. In one embodiment, an antisense polynucleotide molecule is antisense to a coding region of a polynucleotide that encodes LAR, RPTP-σ, or RPTP-δ. The antisense polynucleotide may comprise a sequence that is antisense to a portion of the nucleotide sequence that is unique to LAR, RPTP-σ, or RPTP-δ or may comprise a sequence that is antisense to a portion of the coding sequence that is similar or identical in each of the polynucleotides that encodes LAR, RPTP-σ, or RPTP-δ. The term coding region refers to the region of the nucleotide sequence comprising codons that are translated into amino acid residues. In another embodiment, the antisense nucleic acid molecule is antisense to a “noncoding region” of the coding strand of a nucleotide sequence encoding any one of LAR, RPTP-σ, or RPTP-δ. The term “noncoding region” refers to 5′ and 3′ sequences that flank the coding region that are not translated into amino acids (i.e., also referred to as 5′ and 3′ untranslated regions).

Given the coding strand sequences encoding the RPTPs disclosed herein and available in the art, antisense polynucleotides can be designed according to the rules of Watson and Crick base pairing. The antisense polynucleotide can be complementary to the entire coding region of an RPTP mRNA, for example, or may be an oligonucleotide that is antisense to only a portion of the coding or noncoding region of the RPTP mRNA. For example, the antisense oligonucleotide can be complementary to the region surrounding the translation start site of the RPTP mRNA. An antisense oligonucleotide can be, for example, about 5, 10, 15, 20, 25, 30, 35, 40, 45 or 50 nucleotides or more in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Antisense oligonucleotides are typically designed to resist degradation by endogenous nucleolytic enzymes by using such linkages as phosphorothioate, methylphosphonate, sulfone, sulfate, ketyl, phosphorodithioate, phosphoramidate, phosphate esters, and other such linkages (see, e.g., Agrwal et al., Tetrahedron Lett. 28:3539-42 (1987); Miller et al., J. Am. Chem. Soc. 93:6657-65 (1971); Stec et al., Tetrahedron Lett. 26:2191-2194 (1985); Moody et al., Nucleic Acids Res. 12:4769-82 (1989); Uznanski et al., Nucleic Acids Res. 17:4863-71 (1989); Letsinger et al., Tetrahedron 40:137-43 (1984); Eckstein, Annu. Rev. Biochem. 54:367-402 (1985); Eckstein, Trends Biol. Sci. 14:97-100 (1989); Stein, In: Oligodeoxynucleotides. Antisense Inhibitors of Gene Expression, Cohen, ed., Macmillan Press, London, pp. 97-117 (1989); Jager et al., Biochemistry 27:7237-46 (1988)). Examples of modified nucleotides that can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense polynucleotide (or oligonucleotide) can be produced biologically using an expression vector into which a nucleic acid has been subcloned in an antisense orientation (i.e., RNA transcribed from the inserted nucleic acid will be of an antisense orientation to a target polynucleotide of interest.

An antisense polynucleotide that is specific for one or more polynucleotides that encodes LAR, RPTP-σ, or RPTP-δ is typically administered to a subject or generated in situ such that the antisense polynucleotide hybridizes with or binds to cellular mRNA and/or genomic DNA encoding the RPTP to thereby inhibit expression of the protein, e.g., by inhibiting transcription and/or translation. Hybridization can be by conventional nucleotide complementarity resulting in the formation of a stable duplex, or, for example, when an antisense polynucleotide binds to DNA duplexes, the antisense polynucleotide binds through specific interactions in the major groove of the double helix.

An antisense polynucleotide may be administered to a host or subject by direct injection at a tissue site. Alternatively, antisense polynucleotides can be modified or engineered to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. An antisense polynucleotide can also be delivered to cells using the vectors described herein and used in the art. To achieve sufficient intracellular concentrations of the antisense molecules, a vector may be constructed so that the antisense polynucleotide is placed under the control of a strong pol II or pol III promoter.

In yet another embodiment, the antisense polynucleotide is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett. 215:327-330 (1987)).

In another embodiment, immunoresponsiveness of an immune cell may be altered by contacting a cell that expresses one or more of LAR, RPTP-σ, or RPTP-δ with a ribozyme. A ribozyme is a catalytic RNA molecule with ribonuclease activity that is capable of specifically cleaving a single-stranded nucleic acid, such as an mRNA, to which the ribozyme has a complementary region, resulting in specific inhibition or interference with cellular gene expression. At least five known classes of ribozymes are involved in the cleavage and/or ligation of RNA chains (e.g., hammerhead ribozymes, described in Haselhoff and Gerlach (Nature 334:585-591 (1988)). Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246). Thus, a ribozyme that is specific for an RPTP-encoding nucleic acid can be designed based upon the nucleotide sequence of an RPTP, as described herein and available in the art. For example, a derivative of a Tetrahymena L-19 IVS RNA can be constructed in which the nucleotide sequence of the active site is complementary to the nucleotide sequence to be cleaved in an RPTP-encoding mRNA. (See, e.g., Cech et al. U.S. Pat. No. 4,987,071; and Cech et al. U.S. Pat. No. 5,116,742.) Alternatively, an mRNA molecule that encodes an RPTP can be used to select a catalytic RNA that has a specific ribonuclease activity from a pool of RNA molecules (see, e.g., Bartel et al., Science 261:1411-18 (1993)).

Peptide Nucleic Acids

In another embodiment, peptide nucleic acids (PNAs) can be prepared by modifying the deoxyribose phosphate backbone of a polynucleotide (or a portion thereof) that encodes any one of the RPTPs described herein (see, e.g., Hyrup B. et al., Bioorganic & Medicinal Chemistry 4:5-23) (1996)). The terms “peptide nucleic acid” or “PNA” refers to a nucleic acid mimic, for example, a DNA mimic, in which the deoxyribose phosphate backbone is replaced by a pseudopeptide backbone wherein only the four natural nucleobases are retained. The neutral backbone of a PNA has been shown to allow for specific hybridization to DNA and RNA under conditions of low ionic strength. The synthesis of PNA oligomers can be performed using standard solid phase peptide synthesis protocols (see, e.g., Hyrup B., supra; Perry-O'Keefe et al., Proc. Natl. Acad. Sci. USA 93:14670-75 (1996)). A PNA molecule that is specific for one or more of LAR, RPTP-σ, and RPTP-δ can be used as an antisense or anti-gene agent for sequence-specific modulation of gene expression for example, by inducing transcription or translation arrest or by inhibiting replication.

Aptamers

Aptamers are DNA or RNA molecules, generally single-stranded, that have been selected from random pools based on their ability to bind other molecules, including nucleic acids, proteins, lipids, etc. Unlike antisense polynucleotides, short interfering RNA (siRNA), or ribozymes that bind to a polynucleotide that comprises a sequence that encodes a polypeptide of interest and that alter transcription or translation, aptamers can target and bind to polypeptides. Aptamers may be selected from random or unmodified oligonucleotide libraries by their ability to bind to specific targets, in this instance, LAR, RPTP-δ, and/or RPTP-σ (see, e.g., U.S. Pat. No. 6,867,289; U.S. Pat. No. 5,567,588). Aptamers have capacity to form a variety of two- and three-dimensional structures and have sufficient chemical versatility available within their monomers to act as ligands (i.e., to form specific binding pairs) with virtually any chemical compound, whether monomeric or polymeric. Molecules of any size or composition can serve as targets. An iterative process of in vitro selection may be used to enrich the library for species with high affinity to the target. This process involves repetitive cycles of incubation of the library with a desired target, separation of free oligonucleotides from those bound to the target, and amplification of the bound oligonucleotide subset, such as by using the polymerase chain reaction (PCR). From the selected sub-population of sequences that have high affinity for the target, a sub-population may be subcloned and particular aptamers examined in further detail to identify aptamers that alter a biological function of the target (see, e.g., U.S. Pat. No. 6,699,843).

Aptamers may comprise any deoxyribonucleotide or ribonucleotide or modifications of these bases, such as deoxythiophosphosphate (or phosphorothioate), which have sulfur in place of oxygen as one of the non-bridging ligands bound to the phosphorus. Monothiophosphates αS have one sulfur atom and are thus chiral around the phosphorus center. Dithiophosphates are substituted at both oxygens and are thus achiral. Phosphorothioate nucleotides are commercially available or can be synthesized by several different methods known in the art.

### Antibodies and Antigen-Binding Fragments

Provided herein are antibodies that specifically bind to LAR, RPTP-δ, or to RPTP-σ; antibodies that specifically bind to LAR and RPTP-δ; antibodies that specifically bind to LAR and RPTP-σ; antibodies that specifically bind to RPTP-δ and RPTP-σ; and antibodies that specifically bind to LAR, RPTP-δ, and RPTP-σ, and methods of making and using these antibodies. These specific antibodies may be polyclonal or monoclonal, prepared by immunization of animals and subsequent isolation of the antibody, or the antibodies may be recombinant antibodies. The antibodies described herein are useful for affecting the immunoresponsiveness of an immune cell that expresses at least one of LAR, RPTP-δ, and RPTP-σ. In certain embodiments, the antibodies suppress the immunoresponsiveness of an immune cell that expresses at least one of LAR, RPTP-δ, and RPTP-σ. Such antibodies include those that exhibit a similar effect on the immune cell as the poxvirus protein A41L or 130L. These antibodies are capable of competitively inhibiting binding and/or impairing (i.e., preventing, blocking, decreasing) binding of A41L (or alternatively, 130L) to an immune cell. In one embodiment, an antibody or antigen-binding fragment thereof specifically binds to at least two RPTPs, which may be any two of LAR, RPTP-δ, and RPTP-σ, and competitively inhibits binding of A41L (or 130L) to the at least two RPTP polypeptides. In another embodiment, such an antibody inhibits binding of A41L (or 130L) to an immune cell that expresses any one of LAR, RPTP-δ, and RPTP-σ. Thus, the antibody or antigen-binding fragment thereof suppresses the immunoresponsiveness of the immune cell, which expresses any one of LAR, RPTP-δ, and RPTP-σ. In a particular embodiment, an antibody, or antigen-binding fragment thereof, specifically binds to both RPTP-δ and RPTP-σ and inhibits binding of A41L or 130L to RPTP-δ or to RPTP-σ or to both RPTP-δ and RPTP-σ. In another embodiment, an antibody or antigen-binding fragment thereof specifically binds to all three of LAR, RPTP-δ, and RPTP-σ.

The antibodies described herein may be useful for treating or preventing, inhibiting, slowing the progression of, or reducing the symptoms associated with, an immunological disease or disorder, a cardiovascular disease or disorder, a metabolic disease or disorder, or a proliferative disease or disorder. An immunological disorder includes an inflammatory disease or disorder and an autoimmune disease or disorder. While inflammation or an inflammatory response is a host's normal and protective response to an injury, inflammation can cause undesired damage. For example, atherosclerosis is, at least in part, a pathological response to arterial injury and the consequent inflammatory cascade. Examples of immunological disorders that may be treated with an antibody or antigen-binding fragment thereof described herein include but are not limited to multiple sclerosis, rheumatoid arthritis, systemic lupus erythematosus (SLE), graft versus host disease (GVHD), sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis and other inflammatory and muscle degenerative diseases (e.g., dermatomyositis, polymyositis, juvenile dermatomyositis, inclusion body myositis). A cardiovascular disease or disorder that may be treated, which may include a disease and disorder that is also considered an immunological disease/disorder, includes for example, atherosclerosis, endocarditis, hypertension, or peripheral ischemic disease. A metabolic disease or disorder includes diabetes, obesity, and diseases and disorders associated with abnormal or altered mitochondrial function.

Any one or more of the RPTPs described herein may also be used in methods for screening samples containing antibodies, for example, samples of purified antibodies, antisera, or cell culture supernatants, or any other biological sample that may contain one or more antibodies specific for one or more of the RPTPs. One or more of the RPTPs may also be used in methods for identifying and selecting from a biological sample one or more B cells that are producing an antibody that specifically binds to the one or more of the RPTPs (e.g., plaque forming assays and the like). The B cells may then be used as source of the specific antibody-encoding polynucleotide that can be cloned and/or modified by recombinant molecular biology techniques known in the art and described herein.

As used herein, an antibody is said to be “immunospecific,” “specific for” or to “specifically bind” one or more of LAR, RPTP-δ and RPTP-σ if it reacts at a detectable level with the one or more RPTPs, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and an anti-RPTP antibody specifically binds to one or more RPTPs if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

Binding properties of an antibody to an RPTP described herein may generally be determined and assessed using immunodetection methods including, for example, an enzyme-linked immunosorbent assay (ELISA), immunoprecipitation, immunoblotting, countercurrent immunoelectrophoresis, radioimmunoassays, dot blot assays, inhibition or competition assays, and the like, which may be readily performed by those having ordinary skill in the art (see, e.g., U.S. Pat. Nos. 4,376,110 and 4,486,530; Harlow et al., Antibodies: A Laboratory Manual, Cold Spring Harbor Laboratory (1988)). Immunoassay methods may include controls and procedures to determine whether antibodies bind specifically to LAR, RPTP-δ, and/or RPTP-σ and do not recognize or cross-react with other protein tyrosine phosphatases, particularly other receptor-like protein tyrosine phosphatases. In addition, an immunoassay performed for detection of anti-RPTP (i.e., anti-LAR, anti-RPTP-δ, and/or anti-RPTP-σ) antibodies that are produced in response to immunization of a host with an RPTP conjugated to a particular carrier polypeptide may incorporate the use of RPTP that is conjugated to a different carrier polypeptide than that used for immunization to reduce or eliminate detection of antibodies that bind specifically to the immunizing carrier polypeptide. Alternatively, an RPTP described herein that is not conjugated to a carrier molecule may be used in an immunoassay for detecting immunospecific antibodies.

In certain embodiments, an antibody as described herein is specific for only one of LAR, RPTP-δ, and RPTP-σ. That is, for example, an antibody that specifically binds to LAR does not specifically bind to either RPTP-δ or RPTP-σ; an antibody that specifically binds to RPTP-δ does not specifically bind to LAR or to RPTP-σ; and an antibody that specifically binds to RPTP-σ does not specifically bind to LAR or to RPTP-δ. Such antibodies that specifically bind to only one RPTP described herein bind to an epitope (antigenic determinant) that comprises an amino acid sequence of the RPTP that is not identical or similar to an amino acid sequence present in another RPTP, or such antibodies specifically bind to a conformational epitope that is present in only the RPTP to which the antibody specifically binds. The specificity of an antibody for a particular RPTP may be readily determined using any of the various immunoassays available in the art and described herein.

In other embodiments, an antibody or antigen-binding fragment thereof specifically binds to at least two of LAR, RPTP-δ, and RPTP-σ (i.e., LAR and RPTP-δ; LAR and RPTP-σ, or RPTP-δ and RPTP-σ), and in other embodiments, an antibody or antigen-binding fragment thereof specifically binds to all three RPTPs described herein. An antibody that specifically binds to LAR, RPTP-δ, and RPTP-σ recognizes an epitope (antigenic determinant) that is commonly present in each of the RPTPs. An antigenic determinant or epitope that is common to at least two of LAR, RPTP-δ, and RPTP-σ may comprise an amino acid sequence that is identical or similar in each of the at least two RPTPs, or may comprise a conformational epitope common to at least two of the RPTPs, or may comprise a similar chemical structure, for example, a chemical structure that results from distribution of surface charge(s) of the amino acids that are included in the epitope. By way of example, the amino acid sequence set forth in SEQ ID NO:54 (YSAPANLYV) is common to each of LAR, RPTP-δ, and RPTP-σ. An antibody that binds to an epitope that comprises this amino acid sequence located in the second immunoglobulin-like domain of each RPTP would therefore specifically bind to each of LAR, RPTP-δ, and RPTP-σ.

Antibodies may generally be prepared by any of a variety of techniques known to persons having ordinary skill in the art. See, e.g., Harlow et al., Antibodies: A Laboratory Manual, Cold Spring Harbor Laboratory (1988); Peterson, ILAR J. 46:314-19 (2005)). Any one of the RPTPs described herein, or peptides or fragments thereof, or a cell expressing one or more of the RPTPs may be used as an immunogen for immunizing an animal for production of either polyclonal antibodies or monoclonal antibodies. Fragments of each RPTP that may be used as an immunogen may include larger fragments, such as the extracellular region (which includes the three immunoglobulin (Ig) domains and the fibronectin domains) and the intracellular region (which includes the two phosphatase catalytic domains D1 and D2), or smaller fragments thereof.

An immunogen may comprise a portion of the extracellular region, such as at least one of the Ig domains or a portion thereof or at least one of the fibronectin domains or a portion thereof. RPTP peptide and polypeptide immunogens may be used to generate and/or identify antibodies or antigen-binding fragments thereof that are capable of altering (increasing or decreasing in a statistically significant or biological significant manner, preferably decreasing) the immunoresponsiveness of an immune cell. Exemplary peptide immunogens may comprise 6, 7, 8, 9, 10, 11, 12, 20-25, 21-50, 26-30, 31-40, 41-50, 51-60, 61-70, or 71-75 consecutive amino acids of LAR, RPTP-δ, or RPTP-σ as provided herein (or of a variant thereof). For example, peptides derived from the Ig domains, such as SEQ ID NO:53 (SGALQIEQSEESDQGK); SEQ ID NO:54 (YSAPANLYV); SEQ ID NO:55 (WMLGAEDLTPEDDMPIGR); and SEQ ID NO:56 (NVLELNDVR) of RPTP-δ may be used as immunogens. Examples of peptides derived from the fibronectin III repeats of RPTP-δ include SEQ ID NO:57 (GPPSEPVLTQTSEQAPSSAPR); SEQ ID NO:58 (SPQGLGASTAEISAR); SEQ ID NO:59 (YTAVDGEDDKPHEILGIPSDTTK); SEQ ID NO:60 (VGFGEEMVK); and SEQ ID NO:61 (GPGPYSPSVQFR). Examples of peptides derived from the fibronectin III repeats of RPTP-σ include SEQ ID NO:45 (SIGQGPPSESVVTR); SEQ ID NO:46 (HNVDDSLLTTVGSLLEDETYVR); SEQ ID NO:47 (VLAFTSVGDGPLSDPIQVK); SEQ ID NO:48 (TEVGPGPESSPVVVR); SEQ ID NO:49 (WEPPAGTAEDQVLGYR); and SEQ ID NO:50 (TSVLLSWEFPDNYNSPTPYK). An antibody that specifically binds to an antigenic determinant (epitope) present in the intracellular portion of an RPTP would not be expected to competitively inhibit binding (or impair binding) of a poxvirus polypeptide such as A41L or 130L to the RPTP because the viral polypeptide likely alters an immune response of an immune cell by binding to the extracellular portion of a cell surface antigen such as LAR, RPTP-δ, and/or RPTP-σ. An antibody that specifically binds to the intracellular portion of an RPTP may be used in combination with an antibody (or other agent) that alters immunoresponsiveness of an immune cell and that competitively inhibits binding of A41L or 130L to at least one RPTP. Accordingly, peptides and fragments comprising amino acid sequences from the intracellular domain, particularly the catalytic domains, either D1 or D2, may also be used as immunogens (for example, SEQ ID NO:51 (TEVGPGPESSPVVVR) of RPTP-σ).

RPTP peptides and fragments that are useful as immunogens include portions of an RPTP to which A41L or 130L binds. The RPTP domain that interacts with A41L or 130L may be identified by constructing RPTP extracellular domain polypeptides whereby one or more of the extracellular domains is deleted. By way of example, a fusion polypeptide, for example may exclude the fibronectin domains of an RPTP, and thus comprises only one, two, or three RPTP Ig-like domains. Such a RPTP Ig-like domain polypeptide may be fused to an immunoglobulin Fc polypeptide, or mutein thereof, and comprise the first immunoglobulin-like domain of an RPTP, the first and second immunoglobulin-like domains, the first and third immunoglobulin-like domains, the second or third immunoglobulin-like domains, or all three immunoglobulin-like domains fused to an Fc polypeptide. Such RPTP Ig-like domain polypeptides may also be useful for identifying and determining the extent to which a poxvirus polypeptide binds or a cellular ligand binds to an RPTP immunoglobulin-like domain(s).

One method for determining the amino acid sequence of a poxvirus polypeptide binding site, or a portion of the binding site, of any one of LAR, RPTP-δ, and RPTP-σ, includes peptide mapping techniques. For example, LAR, RPTP-δ, or RPTP-σ peptides may be randomly generated by proteolytic digestion using any one or more of various proteases, the peptides separated and/or isolated (e.g., by gel electrophoresis, column chromatography), followed by determination of which peptide(s) a poxvirus polypeptide, such as A41L or 130L, binds to and then sequence analysis of the peptides. The RPTP peptides may also be generated using recombinant methods described herein and practiced in the art. Peptides randomly generated by recombinant methods may also be used to prepare peptide combinatorial libraries or phage libraries as described herein and in the art. Alternatively, the amino acid sequences of portions of LAR, RPTP-σ, and/or RPTP-δ that interact with a poxvirus polypeptide may be determined by computer modeling of the phosphatase, or of a portion of the phosphatase, for example, the extracellular portion or the Ig domains, and/or x-ray crystallography (which may include preparation and analysis of crystals of the phosphatase only or of the phosphatase-viral polypeptide complex).

Immunogenic peptides of LAR, RPTP-δ, or RPTP-σ may also be determined by computer analysis of the amino acid sequence of the RPTP to determine a hydrophilicity plot. Portions of the RPTP that are accessible to an antibody are most likely portions of the protein that are in contact with the aqueous environment and are hydrophilic. Regions of hydrophilicity can be determined using computer programs available to persons skilled in the art and which assign a “hydrophilic index” to each amino acid in a protein and then plot a profile.

Preparation of an immunogen, particularly polypeptide fragments or peptides, for injection into animals may include covalent coupling of the RPTP fragment or peptide (or variant thereof), to another immunogenic protein, for example, a carrier protein such as keyhole limpet hemocyanin (KLH) or bovine serum albumin (BSA) or the like. A polypeptide or peptide immunogen may include one or more additional amino acids at either the N-terminal or C-terminal end that facilitate the conjugation procedure (e.g., the addition of a cysteine to facilitate conjugation of a peptide to KLH). Other amino acid residues within a polypeptide or peptide may be substituted to prevent conjugation at that particular amino acid position to a carrier polypeptide (e.g., substituting a serine residue for cysteine at internal positions of a polypeptide/peptide) or may be substituted to facilitate solubility or to increase immunogenicity.

An antibody as contemplated and described herein may belong to any immunoglobulin class, for example IgG, IgE, IgM, IgD, or IgA. It may be obtained from or derived from an animal, for example, fowl (e.g., chicken) and mammals, which include but are not limited to a mouse, rat, hamster, rabbit, or other rodent, a cow, horse, sheep, goat, camel, human, or other primate. The antibody may be an internalising antibody. In one such technique, an animal is immunized with an RPTP or fragment thereof as described herein as an antigen to generate polyclonal antisera. Suitable animals include, for example, rabbits, sheep, goats, pigs, cattle, and may also include smaller mammalian species, such as mice, rats, and hamsters, or other species.

Polyclonal antibodies that bind specifically to LAR, RPTP-δ, and/or RPTP-σ can be prepared using methods described herein and practiced by persons skilled in the art (see, for example, Green et al., “Production of Polyclonal Antisera,” in Immunochemical Protocols (Manson, ed.), pages 1-5 (Humana Press 1992); Harlow et al., Antibodies: A Laboratory Manual, Cold Spring Harbor Laboratory (1988); Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995)). Although polyclonal antibodies are typically raised in animals such as rats, mice, rabbits, goats, cattle, or sheep, an anti-RPTP antibody may also be obtained from a subhuman primate. General techniques for raising diagnostically and therapeutically useful antibodies in baboons may be found, for example, in International Patent Application Publication No. WO 91/11465 (1991) and in Losman et al., Int. J. Cancer 46:310, 1990.

In addition, the LAR, RPTP-δ, and/or RPTP-σ polypeptide, fragment or peptide thereof, or a cell expressing one or more of these RPTPs used as an immunogen may be emulsified in an adjuvant. See, e.g., Harlow et al., Antibodies: A Laboratory Manual, Cold Spring Harbor Laboratory (1988). Adjuvants typically used for immunization of non-human animals include but are not limited to Freund's complete adjuvant, Freund's incomplete adjuvant, montanide ISA, Ribi Adjuvant System (RAS) (Corixa Corporation, Seattle, Wash.), and nitrocellulose-adsorbed antigen. The immunogen may be injected into the animal via any number of different routes, including intraperitoneally, intravenously, intramuscularly, intradermally, intraocularly, or subcutaneously. In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera from the collected blood, and analyzing the sera in an immunoassay, such as an ELISA or Ouchterlony diffusion assay, or the like, to determine the specific antibody titer. Once an adequate antibody titer is established, the animals may be bled periodically to accumulate the polyclonal antisera. Polyclonal antibodies that bind specifically to LAR, RPTP-δ, and/or RPTP-σ may then be purified from such antisera, for example, by affinity chromatography using protein A or protein G immobilized on a suitable solid support (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). Alternatively, affinity chromatography may be performed wherein an RPTP or an antibody specific for an Ig constant region of the particular immunized animal species is immobilized on a suitable solid support.

Monoclonal antibodies that specifically bind to LAR, RPTP-δ, and/or RPTP-σ and hybridomas, which are examples of immortal eukaryotic cell lines, that produce monoclonal antibodies having the desired binding specificity, may also be prepared, for example, using the technique of Kohler and Milstein (Nature, 256:495-97 (1976), Eur. J. Immunol. 6:511-19 (1975)) and improvements thereto (see, e.g., Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); and Antibodies: A Laboratory Manual, Harlow and Lane (eds.), Cold Spring Harbor Laboratory Press (1988); see also, e.g., Brand et al., Planta Med. 70:986-92 (2004); Pasqualini et al., Proc. Natl. Acad. Sci. USA 101:257-59 (2004)). An animal, for example, a rat, hamster, or more commonly, a mouse, is immunized with a RPTP immunogen prepared as described above. The presence of specific antibody production may be monitored after the initial injection (injections may be administered by any one of several routes as described herein for generation of polyclonal antibodies) and/or after a booster injection by obtaining a serum sample and detecting the presence of an antibody that binds to LAR, RPTP-δ, and/or RPTP-σ using any one of several immunodetection methods known in the art and described herein.

From animals producing antibodies that bind to LAR, RPTP-δ, and/or RPTP-σ, lymphoid cells, most commonly cells from the spleen or lymph node, are removed to obtain B-lymphocytes, which are lymphoid cells that are antibody-forming cells. The lymphoid cells, typically spleen cells, may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products, e.g., P3X63-Ag 8.653 (ATCC No. CRL 1580); NS0, SP20)). The lymphoid cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Antibodies produced by the cells may be tested for binding activity to LAR, RPTP-δ, and/or RPTP-σ. The hybridomas are cloned (e.g., by limited dilution cloning or by soft agar plaque isolation) and positive clones that produce an antibody specific to the antigen are selected and cultured. Hybridomas producing monoclonal antibodies with high affinity and specificity for LAR, RPTP-δ, and/or RPTP-σ are preferred.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently harvested ascites fluid (usually within 1-3 weeks) by conventional techniques, such as chromatography (e.g., size-exclusion, ion-exchange), gel filtration, precipitation, extraction, or the like (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody, an LAR, RPTP-δ, and/or RPTP-σ or fragment thereof.

An antibody that specifically binds to LAR, RPTP-δ, and/or RPTP-σ may be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein.

For example, human monoclonal antibodies may be obtained from transgenic mice that have been engineered to produce specific human antibodies in response to antigenic challenge. Methods for obtaining human antibodies from transgenic mice are described, for example, by Green et al., Nature Genet. 7:13 (1994); Lonberg et al., Nature 368:856 (1994); Taylor et al., Int. Immun. 6:579 (1994); U.S. Pat. No. 5,877,397; Bruggemann et al., Curr. Opin. Biotechnol. 8:455-58 (1997); Jakobovits et al., Ann. N.Y. Acad. Sci. 764:525-35 (1995). In this technique, elements of the human heavy and light chain locus are artificially introduced by genetic engineering into strains of mice derived from embryonic stem cell lines that contain targeted disruptions of the endogenous murine heavy chain and light chain loci. (See also Bruggemann et al., Curr. Opin. Biotechnol. 8:455-58 (1997)). For example, human immunoglobulin transgenes may be mini-gene constructs, or transloci on yeast artificial chromosomes, which undergo B cell-specific DNA rearrangement and hypermutation in the mouse lymphoid tissue. Human monoclonal antibodies may be obtained by immunizing the transgenic mice, which may then produce human antibodies specific for the antigen. Lymphoid cells of the immunized transgenic mice can be used to produce human antibody-secreting hybridomas according to the methods described herein. Polyclonal sera containing human antibodies may also be obtained from the blood of the immunized animals.

Another method for generating human antigen specific monoclonal antibodies includes immortalizing human peripheral blood cells by EBV transformation. See, e.g., U.S. Pat. No. 4,464,456. Such an immortalized B cell line (or lymphoblastoid cell line) producing a monoclonal antibody that specifically binds to LAR, RPTP-δ, and/or RPTP-σ can be identified by immunodetection methods as provided herein, for example, an ELISA, and then isolated by standard cloning techniques. The stability of the lymphoblastoid cell line producing an anti-LAR, RPTP-δ, and/or RPTP-σ antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B cells with antigen, followed by fusion of primed B cells with a heterohybrid fusion partner. See, e.g., Boerner et al., J. Immunol. 147:86-95 (1991).

In certain embodiments, a B cell that is producing an anti-RPTP antibody is selected, and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)) and described herein. B cells from an immunized animal are isolated from the spleen, lymph node, or peripheral blood sample by selecting a cell that is producing an antibody that specifically binds to LAR, RPTP-δ, and/or RPTP-σ. B cells may also be isolated from humans, for example, from a peripheral blood sample. Methods for detecting single B cells that are producing an antibody with the desired specificity are well known in the art, for example, by plaque formation, fluorescence-activated cell sorting, in vitro stimulation followed by detection of specific antibody, and the like. Methods for selection of specific antibody producing B cells include, for example, preparing a single cell suspension of B cells in soft agar that contains LAR, RPTP-δ, and/or RPTP-σ or a fragment thereof. Binding of the specific antibody produced by the B cell to the antigen results in the formation of a complex, which may be visible as an immunoprecipitate. After the B cells producing the specific antibody are selected, the specific antibody genes may be cloned by isolating and amplifying DNA or mRNA according to methods known in the art and described herein.

Chimeric antibodies, specific for LAR, RPTP-δ, and/or RPTP-σ, including humanized antibodies, may also be generated. A chimeric antibody has at least one constant region domain derived from a first mammalian species and at least one variable region domain derived from a second, distinct mammalian species. See, e.g., Morrison et al., Proc. Natl. Acad. Sci. USA, 81:6851-55 (1984). In one embodiment, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region (see, e.g., Shin et al., Methods Enzymol. 178:459-76 (1989); Walls et al., Nucleic Acids Res. 21:2921-29 (1993)). By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fc receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for LAR, RPTP-δ, and/or RPTP-σ when compared, for example, with either a non-human monoclonal antibody from which an LAR, RPTP-δ, and/or RPTP-σ binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for LAR, RPTP-δ, and/or RPTP-σ, which in some preferred embodiments may be substantially the same affinity for LAR, RPTP-δ, and/or RPTP-σ, and in certain other embodiments may be a greater affinity for LAR, RPTP-δ, and/or RPTP-σ (see, e.g., Jones et al., Nature 321:522-25 (1986); Riechmann et al., Nature 332:323-27 (1988)).

Designing a humanized antibody may therefore include determining CDR loop conformations and structural determinants of the non-human variable regions, for example, by computer modeling, and then comparing the CDR loops and determinants to known human CDR loop structures and determinants (see, e.g., Padlan et al., FASEB 9:133-39 (1995); Chothia et al., Nature, 342:377-83 (1989)). Computer modeling may also be used to compare human structural templates selected by sequence homology with the non-human variable regions (see, e.g., Bajorath et al., Ther. Immunol. 2:95-103 (1995); EP-0578515-A3). If humanization of the non-human CDRs results in a decrease in binding affinity, computer modeling may aid in identifying specific amino acid residues that could be changed by site-directed or other mutagenesis techniques to partially, completely, or supra-optimally (i.e., increase to a level greater than that of the non-humanized antibody) restore affinity. Those having ordinary skill in the art are familiar with these techniques and will readily appreciate numerous variations and modifications to such design strategies.

One such method for preparing a humanized antibody is called veneering. Veneering framework (FR) residues refers to the selective replacement of FR residues from, e.g., a rodent heavy or light chain V region, with human FR residues in order to provide a xenogeneic molecule comprising an antigen-binding site that retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface (see, e.g., Davies et al., Ann. Rev. Biochem. 59:439-73, (1990)). Thus, antigen binding specificity can be preserved in a humanized antibody when the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1991), updates to the Kabat database, and other accessible U.S. and foreign databases (both nucleic acid and protein). Solvent accessibilities of V region amino acids can be deduced from the known three-dimensional structure for human and murine antibody fragments. Initially, the FR amino acid sequence of the variable domains of an antibody molecule of interest are compared with corresponding FR sequences of human variable domains obtained from the above-identified databases and publications. The most homologous human V regions are then compared residue by residue to corresponding murine amino acids. The residues in the murine FR that differ from the human counterpart are replaced by the residues present in the human moiety using recombinant techniques well known in the art. Residue switching is only carried out with moieties that are at least partially exposed (solvent accessible), and care is exercised in the replacement of amino acid residues that may have a significant effect on the tertiary structure of V region domains, such as proline, glycine, and charged amino acids.

In this manner, the resultant “veneered” antigen-binding sites are designed to retain the rodent CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs that are believed to influence the “canonical” tertiary structures of the CDR loops (see, e.g., Chothia et al., Nature, 342:377-383 (1989)). These design criteria are then used to prepare recombinant nucleotide sequences that combine the CDRs of both the heavy and light chain of an antigen-binding site into human-appearing FRs that can be used to transfect mammalian cells for the expression of recombinant human antibodies that exhibit the antigen specificity of the rodent antibody molecule.

For particular uses, antigen-binding fragments of antibodies may be desired. Antibody fragments, F(ab′)₂, Fab, Fab′, Fv, and Fd, can be obtained, for example, by proteolytic hydrolysis of the antibody, for example, pepsin or papain digestion of whole antibodies according to conventional methods. As an illustration, antibody fragments can be produced by enzymatic cleavage of antibodies with pepsin to provide a fragment denoted F(ab′)₂. This fragment can be further cleaved using a thiol reducing agent to produce an Fab′ monovalent fragment. Optionally, the cleavage reaction can be performed using a blocking group for the sulfhydryl groups that result from cleavage of disulfide linkages. As an alternative, an enzymatic cleavage of an antibody using papain produces two monovalent Fab fragments and an Fc fragment (see, e.g., U.S. Pat. No. 4,331,647; Nisonoff et al., Arch. Biochem. Biophys. 89:230 (1960); Porter, Biochem. J. 73:119 (1959); Edelman et al., in Methods in Enzymology 1:422 (Academic Press 1967); Weir, Handbook of Experimental Immunology, Blackwell Scientific, Boston (1986)). The antigen binding fragments may be separated from the Fc fragments by affinity chromatography, for example, using immobilized protein A, protein G, an Fc specific antibody, or immobilized RPTP polypeptide or a fragment thereof. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the RPTP that is recognized by the intact antibody.

An antibody fragment may also be any synthetic or genetically engineered protein that acts like an antibody in that it binds to a specific antigen to form a complex. For example, antibody fragments include isolated fragments consisting of the light chain variable region, Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. The antibody comprises at least one variable region domain. The variable region domain may be of any size or amino acid composition and will generally comprise at least one hypervariable amino acid sequence responsible for antigen binding and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding antigen with acceptable affinity. Alternatively, the V region domain may be dimeric and contain V_{H}-V_{H}, V_{H}-V_{L}, or V_{L}-V_{L}, dimers. Preferably, the V region dimer comprises at least one V_{H} and at least one V_{L} chain that are non-covalently associated (hereinafter referred to as Fᵥ). If desired, the chains may be covalently coupled either directly, for example via a disulfide bond between the two variable domains, or through a linker, for example a peptide linker, to form a single chain Fv (scFᵥ).

A minimal recognition unit is an antibody fragment comprising a single complementarity-determining region (CDR). Such CDR peptides can be obtained by constructing polynucleotides that encode the CDR of an antibody of interest. The polynucleotides are prepared, for example, by using the polymerase chain reaction to synthesize the variable region using mRNA isolated from or contained within antibody-producing cells as a template according to methods practiced by persons skilled in the art (see, for example, Larrick et al., Methods: A Companion to Methods in Enzymology 2:106, (1991); Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). Alternatively, such CDR peptides and other antibody fragment can be synthesized using an automated peptide synthesizer.

According to certain embodiments, non-human, human, or humanized heavy chain and light chain variable regions of any of the Ig molecules described herein may be constructed as scFv polypeptide fragments (single chain antibodies). See, e.g., Bird et al., Science 242:423-426 (1988); Huston et al., Proc. Natl. Acad. Sci. USA 85:5879-83 (1988)). Multi-functional scFv fusion proteins may be generated by linking a polynucleotide sequence encoding an scFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., J. Immunol. Methods 188:1-7 (1995). Other examples of effector proteins are enzymes. As a non-limiting example, such an enzyme may provide a biological activity for therapeutic purposes (see, e.g., Siemers et al., Bioconjug. Chem. 8:510-19 (1997)), or may provide a detectable activity, such as horseradish peroxidase-catalyzed conversion of any of a number of well-known substrates into a detectable product, for diagnostic uses.

The scFv may, in certain embodiments, be fused to peptide or polypeptide domains that permit detection of specific binding between the fusion protein and antigen (e.g., one or more of the RPTPs described herein). For example, the fusion polypeptide domain may be an affinity tag polypeptide. Binding of the scFv fusion protein to a binding partner (e.g., one or more of the RPTPs or fragment thereof described herein) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., J. Biotechnol. 65:225 (1998) and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support. An scFv fusion protein comprising an RPTP-specific immunoglobulin-derived polypeptide may be fused to another polypeptide such as an effector peptide having desirable affinity properties (see, e.g., U.S. Pat. No. 5,100,788; WO 89/03422; U.S. Pat. No. 5,489,528; U.S. Pat. No. 5,672,691; WO 93/24631; U.S. Pat. No. 5,168,049; U.S. Pat. No. 5,272,254; EP 511,747). As provided herein, scFv polypeptide sequences may be fused to fusion polypeptide sequences, including effector protein sequences, that may include full-length fusion polypeptides and that may alternatively contain variants or fragments thereof.

Antibodies may also be identified and isolated from human immunoglobulin phage libraries, from rabbit immunoglobulin phage libraries, from mouse immunoglobulin phage libraries, and/or from chicken immunoglobulin phage libraries (see, e.g., Winter et al., Annu. Rev. Immunol. 12:433-55 (1994); Burton et al., Adv. Immunol. 57:191-280 (1994); U.S. Pat. No. 5,223,409; Huse et al., Science 246:1275-81 (1989); Schlebusch et al., Hybridoma 16:47-52 (1997) and references cited therein; Rader et al., J. Biol. Chem. 275:13668-76 (2000); Popkov et al., J. Mol. Biol. 325:325-35 (2003); Andris-Widhopf et al., J. Immunol. Methods 242:159-31 (2000)). Antibodies isolated from non-human species or non-human immunoglobulin libraries may be genetically engineered according to methods described herein and known in the art to “humanize” the antibody or fragment thereof. Immunoglobulin variable region gene combinatorial libraries may be created in phage vectors that can be screened to select Ig fragments (Fab, Fv, scFv, or multimers thereof) that bind specifically to an RPTP described herein (see, e.g., U.S. Pat. No. 5,223,409; Huse et al., Science 246:1275-81 (1989); Sastry et al., Proc. Natl. Acad. Sci. USA 86:5728-32 (1989); Alting-Mees et al., Strategies in Molecular Biology 3:1-9 (1990); Kang et al., Proc. Natl. Acad. Sci. USA 88:4363-66 (1991); Hoogenboom et al., J. Molec. Biol. 227:381-388 (1992); Schlebusch et al., Hybridoma 16:47-52 (1997) and references cited therein; U.S. Pat. No. 6,703,015).

For example, a library containing a plurality of polynucleotide sequences encoding Ig variable region fragments may be inserted into the genome of a filamentous bacteriophage, such as M13 or a variant thereof, in frame with the sequence encoding a phage coat protein such as gene III or gene VIII. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B cell population and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high-level expression of monoclonal antibody fragments from E. coli.

Phage that display an Ig fragment (e.g., an Ig V-region or Fab) that binds to LAR, RPTP-δ, and/or RPTP-σ may be selected by mixing the phage library with LAR, RPTP-δ, and/or RPTP-σ or a fragment thereof, or by contacting the phage library with such polypeptide or peptide molecules immobilized on a solid matrix under conditions and for a time sufficient to allow binding. Unbound phage are removed by a wash, and specifically bound phage (i.e., phage that contain an RPTP specific Ig fragment) are then eluted (see, e.g., Messmer et al., Biotechniques 30:798-802 (2001)). Eluted phage may be propagated in an appropriate bacterial host, and generally, successive rounds of RPTP binding and elution can be repeated to increase the yield of phage expressing the RPTP-specific immunoglobulin.

Phage display techniques may also be used to select Ig fragments or single chain antibodies that bind to LAR, RPTP-δ, and/or RPTP-σ. For examples of suitable vectors having multicloning sites into which candidate nucleic acid molecules (e.g., DNA) encoding such antibody fragments or related peptides may be inserted, see, e.g., McLafferty et al., Gene 128:29-36 (1993); Scott et al., Science 249:386-90 (1990); Smith et al., Meth. Enzymol. 217:228-57 (1993); Fisch et al., Proc. Natl. Acad. Sci. USA 93:7761-66 (1996)). The inserted DNA molecules may comprise randomly generated sequences, or may encode variants of a known peptide or polypeptide domain (such as A41L) that specifically binds to LAR, RPTP-δ, and/or RPTP-σ. Generally, the nucleic acid insert encodes a peptide of up to 60 amino acids, or may encode a peptide of 3 to 35 amino acids, or may encode a peptide of 6 to 20 amino acids. The peptide encoded by the inserted sequence is displayed on the surface of the bacteriophage. Phage expressing a binding domain for the RPTP may be selected on the basis of specific binding to an immobilized RPTP or a fragment thereof. Well-known recombinant genetic techniques may be used to construct fusion proteins containing the fragment. For example, a polypeptide may be generated that comprises a tandem array of two or more similar or dissimilar affinity selected RPTP binding peptide domains, in order to maximize binding affinity for LAR, RPTP-δ, and/or RPTP-σ of the resulting product.

Combinatorial mutagenesis strategies using phage libraries may also be used for humanizing non-human variable regions (see, e.g., Rosok et al., J. Biol. Chem. 271:22611-18 (1996); Rader et al., Proc. Natl. Acad. Sci. USA 95:8910-15 (1998)). Humanized variable regions that have binding affinity that is minimally reduced or that is comparable to the non-human variable region can be selected, and the nucleotide sequences encoding the humanized variable regions may be determined by standard techniques (see, Sambrook et al., Molecular Cloning: A Laboratory Manual, Cold Spring Harbor Press (2001)). The affinity selected Ig-encoding sequence may then be cloned into another suitable vector for expression of the Ig fragment or, optionally, may be cloned into a vector containing Ig constant regions, for expression of whole immunoglobulin chains.

Similarly, portions or fragments, such as Fab and Fv fragments, of RPTP specific antibodies may be constructed using conventional enzymatic digestion or recombinant DNA techniques to incorporate the variable regions of a gene that encodes an antibody specific for LAR, RPTP-δ, and/or RPTP-σ. Within one embodiment, in a hybridoma the variable regions of a gene expressing a monoclonal antibody of interest are amplified using nucleotide primers. These primers may be synthesized by one of ordinary skill in the art, or may be purchased from commercially available sources (see, e.g., Stratagene (La Jolla, Calif.), which sells primers for amplifying mouse and human variable regions. The primers may be used to amplify heavy or light chain variable regions, which may then be inserted into vectors such as ImmunoZAP™ H or ImmunoZAP™ L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426 (1988)). In addition, such techniques may be used to humanize a non-human antibody V region without altering the binding specificity of the antibody.

In certain other embodiments, RPTP-specific antibodies are multimeric antibody fragments. Useful methodologies are described generally, for example in Hayden et al., Curr Opin. Immunol. 9:201-12 (1997) and Coloma et al., Nat. Biotechnol. 15:159-63 (1997). For example, multimeric antibody fragments may be created by phage techniques to form miniantibodies (U.S. Pat. No. 5,910,573) or diabodies (Holliger et al., Cancer Immunol. Immunother. 45:128-30 (1997)). Multimeric fragments may be generated that are multimers of an RPTP-specific Fv.

Multimeric antibodies include bispecific and bifunctional antibodies comprising a first Fv specific for an antigen associated with a second Fv having a different antigen specificity (see, e.g., Drakeman et al., Expert Opin. Investig. Drugs 6:1169-78 (1997); Koelemij et al., J. Immunother. 22:514-24 (1999); Marvin et al., Acta Pharmacol. Sin. 26:649-58 (2005); Das et al., Methods Mol. Med. 109:329-46 (2005)). For example, in one embodiment, a bispecific antibody comprises an Fv, or other antigen-binding fragment described herein, that specifically binds to LAR and comprises an Fv, or other antigen-binding fragment, that specifically binds to RPTP-σ. Similarly, in another embodiment, a bispecific antibody comprises an Fv, or other antigen-binding fragment described herein, that specifically binds to LAR and comprises an Fv, or other antigen-binding fragment, that specifically binds to RPTP-δ. In still another embodiment, a bispecific antibody comprises an Fv, or other antigen-binding fragment described herein, that specifically binds to RPTP-σ and comprises an Fv, or other antigen-binding fragment, that specifically binds to RPTP-δ. In other certain embodiments, a multivalent antibody or bispecific antibody comprises an Fv, or other antigen-binding fragment, that specifically binds to at least one of LAR, RPTP-δ, and RPTP-σ, and further comprises an Fv, or other antigen-binding fragment, that is specific for a non-PTP polypeptide, such as for example, a cell surface antigen that when bound by a specific antibody contributes to, facilitates, or is capable of altering (suppressing or enhancing) immunoresponsiveness of an immune cell.

Introducing amino acid mutations into RPTP-binding immunoglobulin molecules may be useful to increase the specificity or affinity for the RPTP, or to alter an effector function. Immunoglobulins with higher affinity for LAR, RPTP-δ, and/or RPTP-σ may be generated by site-directed mutagenesis of particular residues. Computer assisted three-dimensional molecular modeling may be used to identify the amino acid residues to be changed in order to improve affinity for the RPTP (see, e.g., Mountain et al., Biotechnol. Genet. Eng. Rev. 10:1-142 (1992)). Alternatively, combinatorial libraries of CDRs may be generated in M13 phage and screened for immunoglobulin fragments with improved affinity (see, e.g., Glaser et al., J. Immunol. 149:3903-13 (1992); Barbas et al., Proc. Natl. Acad. Sci. USA 91:3809-13 (1994); U.S. Pat. No. 5,792,456).

In certain embodiments, the antibody may be genetically engineered to have an altered effector function. For example, the antibody may have an altered capability (increased or decreased in a biologically or statistically significant manner) to mediate complement dependent cytotoxicity (CDC) or antibody dependent cellular cytotoxicity (ADCC) or an altered capability for binding to effector cells via Fc receptors present on the effector cells. Effector functions may be altered by site-directed mutagenesis (see, e.g., Duncan et al., Nature 332:563-64 (1988); Morgan et al., Immunology 86:319-24 (1995); Eghtedarzedeh-Kondri et al., Biotechniques 23:830-34 (1997)). For example, mutation of the glycosylation site on the Fc portion of the immunoglobulin may alter the capability of the immunoglobulin to fix complement (see, e.g., Wright et al., Trends Biotechnol. 15:26-32 (1997)). Other mutations in the constant region domains may alter the ability of the immunoglobulin to fix complement or to effect ADCC (see, e.g., Duncan et al., Nature 332:563-64 (1988); Morgan et al., Immunology 86:319-24 (1995); Sensel et al., Mol. Immunol. 34:1019-29 (1997)). (See also, e.g., U.S. Patent Publication Nos. 2003/0118592; 2003/0133939).

The nucleic acid molecules encoding an antibody or fragment thereof that specifically binds an RPTP, as described herein, may be propagated and expressed according to any of a variety of well-known procedures for nucleic acid excision, ligation, transformation, and transfection. Thus, in certain embodiments expression of an antibody fragment may be preferred in a prokaryotic host cell, such as Escherichia coli (see, e.g., Pluckthun et al., Methods Enzymol. 178:497-515 (1989)). In certain other embodiments, expression of the antibody or an antigen-binding fragment thereof may be preferred in a eukaryotic host cell, including yeast (e.g., Saccharomyces cerevisiae, Schizosaccharomyces pombe, and Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the RPTP binding antibody (or fragment thereof) may be inserted. The regulatory elements will vary according to the particular host.

One or more replicable expression vectors containing a polynucleotide encoding a variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the polynucleotide sequence in each vector should include appropriate regulatory sequences, particularly a promoter and leader sequence operatively linked to the variable domain sequence. Particular methods for producing antibodies in this way are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto.

Site directed mutagenesis of an immunoglobulin variable (V region), framework region, and/or constant region may be performed according to any one of numerous methods described herein and practiced in the art (Kramer et al., Nucleic Acids Res. 12:9441 (1984); Kunkel Proc. Natl. Acad. Sci. USA 82:488-92 (1985); Kunkel et al., Methods Enzymol. 154:367-82 (1987)). Random mutagenesis methods to identify residues that are either important to binding to an RPTP (LAR, RPTP-δ, and/or RPTP-σ) or that do not alter binding of the antigen to the antibody when altered can also be performed according to procedures that are routinely practiced by a person skilled in the art (e.g., alanine scanning mutagenesis; error prone polymerase chain reaction mutagenesis; and oligonucleotide-directed mutagenesis (see, e.g., Sambrook et al. Molecular Cloning: A Laboratory Manual, 3rd ed., Cold Spring Harbor Laboratory Press, NY (2001))). Additionally, numerous publications describe techniques suitable for the preparation of antibodies by manipulation of DNA, creation of expression vectors, and transformation of appropriate cells (Mountain et al., in Biotechnology and Genetic Engineering Reviews (ed. Tombs, M P, 10, Chapter 1, Intercept, Andover, UK (1992)); International Patent Publication No. WO 91/09967).

The antibodies and antigen-binding fragments thereof that specifically bind to LAR, RPTP-δ, and/or RPTP-σ may also be useful as reagents for immunochemical analyses to detect the presence of one or more of the RPTPs in a biological sample. In certain embodiments, an antibody that specifically binds to at least one of LAR, RPTP-δ, and RPTP-σ may be used to detect expression of the at least one RPTP. In certain particular embodiments, one antibody or a panel of antibodies may be exposed to cells that express an RPTP, and expression of the RPTP may be determined by detection using another RPTP specific antibody that binds to a different epitope than the antibody or antibodies initially permitted to interact with the cells.

For such a purpose an RPTP-binding immunoglobulin (or fragment thereof) as described herein may contain a detectable moiety or label such as an enzyme, cytotoxic agent, or other reporter molecule, including a dye, radionuclide, luminescent group, fluorescent group, or biotin, or the like. The RPTP-specific immunoglobulin or fragment thereof may be radiolabeled for diagnostic or therapeutic applications. Techniques for radiolabeling of antibodies are known in the art (see, e.g., Adams, In Vivo 12:11-21 (1998); Hiltunen, Acta Oncol. 32:831-9 (1993)). The effector or reporter molecules may be attached to the antibody through any available amino acid side-chain, terminal amino acid, or carbohydrate functional group located in the antibody, provided that the attachment or attachment process does not adversely affect the binding properties such that the usefulness of the molecule is abrogated. Particular functional groups include, for example, any free amino, imino, thiol, hydroxyl, carboxyl, or aldehyde group. Attachment of the antibody or antigen-binding fragment thereof and the effector and/or reporter molecule(s) may be achieved via such groups and an appropriate functional group in the effector or reporter molecule. The linkage may be direct or indirect through spacing or bridging groups (see, e.g., International Patent Application Publication Nos. WO 93/06231, WO 92/22583, WO 90/091195, and WO 89/01476; see also, e.g., commercial vendors such as Pierce Biotechnology, Rockford, Ill.).

As provided herein and according to methodologies well known in the art, polyclonal and monoclonal antibodies may be used for the affinity isolation of LAR, RPTP-δ, and/or RPTP-σ and fragments thereof (see, e.g., Hermanson et al., Immobilized Affinity Ligand Techniques, Academic Press, Inc. New York, (1992)). Briefly, an antibody (or antigen-binding fragment thereof) may be immobilized on a solid support material, which is then contacted with a sample that contains an RPTP. The sample interacts with the immobilized antibody under conditions and for a time that are sufficient to permit binding of the RPTP to the immobilized antibody; non-binding components (that is, those components unrelated to the RPTP) of the sample are removed; and then the RPTP is released from the immobilized antibody using an appropriate eluting solution.

In certain embodiments, anti-idiotype antibodies that recognize and bind specifically to an antibody (or antigen-binding fragment thereof) that specifically binds to LAR, RPTP-δ, and/or RPTP-σ are provided, and methods for using these anti-idiotype antibodies are also provided. Anti-idiotype antibodies may be generated as polyclonal antibodies or as monoclonal antibodies by the methods described herein, using an anti-LAR, anti-RPTP-δ, or anti-RPTP-σ antibody (or antigen-binding fragment thereof) as immunogen. Anti-idiotype antibodies or fragments thereof may also be generated by any of the recombinant genetic engineering methods described above or by phage display selection. Anti-idiotype antibodies may be further engineered to provide a chimeric or humanized anti-idiotype antibody, according to the description provided in detail herein. An anti-idiotype antibody may bind specifically to the antigen-binding site of the anti-RPTP antibody such that binding of the antibody to the RPTP is competitively inhibited. Alternatively, an anti-idiotype antibody as provided herein may not competitively inhibit binding of an anti-RPTP antibody to the RPTP.

In one embodiment, an anti-idiotype antibody may be used to alter the immunoresponsiveness of an immune cell. In certain embodiments, an anti-idiotype antibody may be used to suppress the immunoresponsiveness of an immune cell and to treat an immunological disease or disorder. An anti-idiotype antibody specifically binds to an antibody that specifically binds to LAR, RPTP-δ, and/or RPTP-σ, and the antigen-binding site of the anti-idiotype antibody mimics the epitope of the RPTP, that is, the anti-idiotype antibody will bind to cognate ligands as well as antibodies that specifically bind to the RPTP. Thus, an anti-idiotype antibody may be useful for preventing, blocking, or reducing binding of a cognate ligand that when such ligand binds to an RPTP, it stimulates, induces, or enhances the immunoresponsiveness of an immune cell.

Anti-idiotype antibodies are also useful for immunoassays to determine the presence of anti-RPTP antibodies in a biological sample. For example, immunoassays, such as an ELISA and other assays described herein that are practiced by persons skilled in the art, may be used to determine the presence of an immune response induced by administering (i.e., immunizing) a host with an RPTP polypeptide or fragment thereof as described herein.

In certain embodiments, an antibody specific for LAR, RPTP-δ, and/or RPTP-σ may be an antibody or antigen-binding fragment thereof that is expressed as an intracellular protein. Such intracellular antibodies are also referred to as intrabodies and may comprise an Fab fragment, a Fv fragment, a scFv molecule, an scFv-Fc fusion antibody, or a bispecific antibody, all of which may be made as described herein and according to methods practiced in the art (see, e.g., Lobato et al., Curr. Mol. Med. 4:519-28 (2004); Strube et al., Methods 34:179-83 (2004); Lecerf et al., Proc. Natl. Acad. Sci. USA 98:4764-49 (2001); (Weisbart et al., Int. J. Oncol. 25:1113-18 (2004)). An antibody that would be useful in the form of an intrabody includes an antibody that specifically binds to the intracellular portion of an RPTP. For example, an antibody that bound to an epitope within a region of the intracellular portion of LAR, RPTP-δ, and/or RPTP-σ, for example, which includes the catalytic domains D1 and D2 and a region comprising a peptide having the sequence set forth in SEQ ID NO:51.

The framework regions flanking the CDR regions can be modified to improve expression levels, stability, and/or solubility of an intrabody in an intracellular reducing environment (see, e.g., Auf der Maur et al., Methods 34:215-24 (2004); Strube et al., supra; Worn et al., J. Biol. Chem. 275:2795-803 (2000)). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell Biol. 15:1182-91 (1995); Lener et al., Eur. J. Biochem. 267:1196-205 (2000); Popkov et al., Cancer Res. 65:972-81 (2005)). Various types of intrabodies have been investigated as therapeutic agents for treating cancer (see, e.g., Weisbart et al., supra; Popkov et al., supra; Krauss et al., Breast Dis. 11:113-24 (1999)) and for treating neurodegenerative diseases such as Parkinson's disease (Zhou et al., Mol. Ther. 10:1023-31 (2004)) and Huntington's disease (Murphy et al., Brain Res. Mol. Brain Res. 121:141-45 (2004); Colby et al., J. Mol. Biol. 342:901-12 (2004); Colby et al., Proc. Natl. Acad. Sci. USA 101: 17616-21 (2004), Erratum in Proc. Natl. Acad. Sci. USA 102:955 (2005)). An intrabody may be introduced into a cell by a variety of techniques available to the skilled artisan including via a gene therapy vector, a lipid mixture (e.g., Provectin™ manufactured by Imgenex Corporation, San Diego, Calif.), photochemical internalization methods, or other methods known in the art.

### Expression of A41L, 130L, RPTPs, and Polypeptide Agents

The polypeptides described herein including A41L, 130L, RPTPs (LAR, RPTP-δ, and RPTP-σ) and fusion polypeptides (e.g., peptide-IgFc fusion polypeptides, RPTP Ig domain-Fc fusion polypeptides) may be expressed using vectors and constructs, particularly recombinant expression constructs, that include any polynucleotide encoding such polypeptides. Host cells are genetically engineered with vectors and/or constructs to produce these polypeptides and fusion proteins, or fragments or variants thereof, by recombinant techniques. Each of the polypeptides and fusion polypeptides described herein can be expressed in mammalian cells, yeast, bacteria, or other cells under the control of appropriate promoters. Cell-free translation systems can also be employed to produce such proteins using RNAs derived from DNA constructs. Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

Generally, recombinant expression vectors include origins of replication, selectable markers permitting transformation of the host cell, for example, the ampicillin resistance gene of E. coli and S. cerevisiae TRP1 gene, and a promoter derived from a highly expressed gene to direct transcription of a downstream structural sequence. Promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences.

Optionally, a heterologous sequence can encode a fusion protein that includes an amino terminal or carboxy terminal identification peptide or polypeptide that imparts desired characteristics, e.g., that stabilizes the produced polypeptide or that simplifies purification of the expressed recombinant product. Such identification peptides include a polyhistidine tag (his tag) or FLAG® epitope tag (DYKDDDDK, SEQ ID NO:62), beta-galatosidase, alkaline phosphatase, GST, or the XPRESS™ epitope tag (DLYDDDDK, SEQ ID NO:63; Invitrogen Life Technologies, Carlsbad, Calif.) and the like (see, e.g., U.S. Pat. No. 5,011,912; Hopp et al., (Bio/Technology 6:1204 (1988)). The affinity sequence may be supplied by a vector, such as, for example, a hexa-histidine tag that is provided in pBAD/His (Invitrogen). Alternatively, the affinity sequence may be added either synthetically or engineered into the primers used to recombinantly generate the nucleic acid coding sequence (e.g., using the polymerase chain reaction).

Host cells containing described recombinant expression constructs may be genetically engineered (transduced, transformed, or transfected) with the vectors and/or expression constructs (for example, a cloning vector, a shuttle vector, or an expression construct). The vector or construct may be in the form of a plasmid, a viral particle, a phage, etc. The engineered host cells can be cultured in conventional nutrient media modified as appropriate for activating promoters, selecting transformants, or amplifying particular genes or encoding-nucleotide sequences. Selection and maintenance of culture conditions for particular host cells, such as temperature, pH and the like, will be readily apparent to the ordinarily skilled artisan. Preferably the host cell can be adapted to sustained propagation in culture to yield a cell line according to art-established methodologies. In certain embodiments, the cell line is an immortal cell line, which refers to a cell line that can be repeatedly (at least ten times while remaining viable) passaged in culture following log-phase growth. In other embodiments the host cell used to generate a cell line is a cell that is capable of unregulated growth, such as a cancer cell, or a transformed cell, or a malignant cell.

Useful bacterial expression constructs are constructed by inserting into an expression vector a structural DNA sequence encoding a desired protein together with suitable translation initiation and termination signals in operable reading phase with a functional promoter. The construct may comprise one or more phenotypic selectable markers and an origin of replication to ensure maintenance of the vector construct and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. Any other plasmid or vector may be used as long as they are replicable and viable in the host. Thus, for example, the nucleic acids as provided herein may be included in any one of a variety of expression vector constructs as a recombinant expression construct for expressing a polypeptide. Such vectors and constructs include chromosomal, nonchromosomal, and synthetic DNA sequences, e.g., bacterial plasmids; phage DNA; baculovirus; yeast plasmids; vectors derived from combinations of plasmids and phage DNA; viral DNA, such as vaccinia, adenovirus, fowl pox virus, and pseudorabies. However, any other vector may be used for preparation of a recombinant expression construct as long as it is replicable and viable in the host.

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. Numerous standard techniques are described, for example, in Ausubel et al. (Current Protocols in Molecular Biology (Greene Publ. Assoc. Inc. & John Wiley & Sons, Inc., 1993)); Sambrook et al. (Molecular Cloning: A Laboratory Manual, 3rd Ed., (Cold Spring Harbor Laboratory 2001)); Maniatis et al. (Molecular Cloning, (Cold Spring Harbor Laboratory 1982)), and elsewhere.

The DNA sequence encoding a polypeptide in the expression vector is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P_{L} promoter, and other promoters known to control expression of genes in prokaryotic or eukaryotic cells or their viruses. Promoter regions can be selected from any desired gene using CAT (chloramphenicol transferase) vectors or other vectors with selectable markers. Particular bacterial promoters include lac, lacZ, T3, T5, T7, gpt, lambda P_{R}, P_{L}, and trp. Eukaryotic promoters include CMV immediate early, HSV thymidine kinase, early and late SV40, LTRs from retroviruses, and mouse metallothionein-I. Selection of the appropriate vector and promoter and preparation of certain recombinant expression constructs comprising at least one promoter or regulated promoter operatively linked to a nucleic acid described herein is well within the level of ordinary skill in the art.

Design and selection of inducible, regulated promoters and/or tightly regulated promoters are known in the art and will depend on the particular host cell and expression system. The pBAD Expression System (Invitrogen Life Technologies, Carlsbad, Calif.) is an example of a tightly regulated expression system that uses the E. coli arabinose operon (P_{BAD} or P_{ARA}) (see Guzman et al., J. Bacteriology 177:4121-30 (1995); Smith et al., J. Biol. Chem. 253:6931-33 (1978); Hirsh et al., Cell 11:545-50 (1977)), which controls the arabinose metabolic pathway. A variety of vectors employing this system are commercially available. Other examples of tightly regulated promoter-driven expression systems include PET Expression Systems (see U.S. Pat. No. 4,952,496) available from Stratagene (La Jolla, Calif.) or tet-regulated expression systems (Gossen et al., Proc. Natl. Acad. Sci. USA 89:5547-51 (1992); Gossen et al., Science 268:1766-69 (1995)). The pLP-TRE2 Acceptor Vector (BD Biosciences Clontech, Palo Alto, Calif.) is designed for use with CLONTECH's Creator™ Cloning Kits to rapidly generate a tetracycline-regulated expression construct for tightly controlled, inducible expression of a gene of interest using the site-specific Cre-lox recombination system (see, e.g., Sauer, Methods 14:381-92 (1998); Furth, J. Mamm. Gland Biol. Neoplas. 2:373 (1997)), which may also be employed for host cell immortalization (see, e.g., Cascio, Artif. Organs 25:529 (2001)).

The vector may be a viral vector such as a retroviral vector. For example, retroviruses from which the retroviral plasmid vectors may be derived include, but are not limited to, Moloney Murine Leukemia Virus, spleen necrosis virus, Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. A viral vector also includes one or more promoters. Suitable promoters that may be employed include, but are not limited to, the retroviral LTR; the SV40 promoter; and the human cytomegalovirus (CMV) promoter described in Miller et al., Biotechniques 7:980-990 (1989), or any other promoter (e.g., eukaryotic cellular promoters including, for example, the histone, pol III, and β-actin promoters). Other viral promoters that may be employed include, but are not limited to, adenovirus promoters, thymidine kinase (TK) promoters, and B19 parvovirus promoters.

The retroviral plasmid vector is employed to transduce packaging cell lines (e.g., PE501, PA317, ψ-2, ψ-AM, PA12, T19-14X, VT-19-17-H2, ψCRE, ψCRIP, GP+E-86, GP+envAm12, DAN; see also, e.g., Miller, Human Gene Therapy, 1:5-14 (1990)) to form producer cell lines. The vector may transduce the packaging cells through any means known in the art, such as, for example, electroporation, the use of liposomes, and calcium phosphate precipitation. The producer cell line generates infectious retroviral vector particles that include the nucleic acid sequence(s) encoding the polypeptides or fusion proteins described herein. Such retroviral vector particles then may be employed, to transduce eukaryotic cells, either in vitro or in vivo. Eukaryotic cells that may be transduced include, for example, embryonic stem cells, embryonic carcinoma cells, hematopoietic stem cells, hepatocytes, fibroblasts, myoblasts, keratinocytes, endothelial cells, bronchial epithelial cells, and other culture-adapted cell lines.

As another example, host cells transduced by a recombinant viral construct directing the expression of polypeptides or fusion proteins may produce viral particles containing expressed polypeptides or fusion proteins that are derived from portions of a host cell membrane incorporated by the viral particles during viral budding. The polypeptide-encoding nucleic acid sequences may be cloned into a baculovirus shuttle vector, which is then recombined with a baculovirus to generate a recombinant baculovirus expression construct that is used to infect, for example, Sf9 host cells (see, e.g., Baculovirus Expression Protocols, Methods in Molecular Biology Vol. 39, Richardson, Ed. (Human Press 1995); Piwnica-Worms, “Expression of Proteins in Insect Cells Using Baculoviral Vectors,” Section II, Chapter 16 in Short Protocols in Molecular Biology, 2^{nd} Ed., Ausubel et al., eds., (John Wiley & Sons 1992), pages 16-32 to 16-48).

Methods for Identifying and Characterizing Agents that Alter Immunoresponsiveness of an Immune Cell

Methods are provided herein for identifying or selecting an agent that alters (suppresses or enhances in a statistically significant or biologically significant manner, preferably suppresses) immunoresponsiveness of an immune cell or for determining the capability of an agent described herein to alter the immunoresponsiveness of an immune cell. In one embodiment, a method is provided for identifying an agent that suppresses immunoresponsiveness of an immune cell comprises contacting (mixing, combining, or in some manner permitting interaction of) (1) a candidate agent; (2) an immune cell that expresses at least one of the RPTPs, LAR, RPTP-δ, and RPTP-σ; and (3) a poxvirus polypeptide such as A41L or 130L, under conditions and for a time sufficient to permit interaction between the at least one RPTP and the poxvirus polypeptide, and then determining the level of binding of the poxvirus polypeptide (i.e., A41L or 130L) to the immune cell in the presence and absence of the candidate agent. A decrease in binding of the poxvirus polypeptide to the immune cell in the presence of the candidate agent indicates that the candidate agent suppresses immunoresponsiveness of the immune cell. In certain embodiments, an immune cell expresses at least two of LAR, RPTP-δ, and RPTP-σ (such as LAR and RPTP-δ; LAR and RPTP-σ, and RPTP-δ and RPTP-σ) and in other particular embodiments, an immune cell expresses all three RPTPs. The immune cell may be present in or isolated from a biological sample as described herein. For example, the immune cell may be obtained from a primary or long-term cell culture or may be present in or isolated from a biological sample obtained from a subject (human or non-human animal).

In another embodiment, a method is provided for identifying an agent that inhibits binding of a poxvirus polypeptide, such as A41L or 130L, to at least two RPTPs (that is, at least two of LAR, RPTP-δ, and RPTP-σ). The method comprises contacting (mixing, combining, or in some manner permitting interaction among) (1) a candidate agent; (2) a biological sample comprising at least two RPTP polypeptides selected from (i) LAR; (ii) RPTP-σ; and (iii) RPTP-δ; and (3) the poxvirus polypeptide, under conditions and for a time sufficient to permit interaction between the at least two RPTP polypeptides and the poxvirus polypeptide. The level of binding of the poxvirus polypeptide to the at least two RPTP polypeptides is then determined in the presence of the candidate agent and compared with the level of binding of the poxvirus polypeptide to each of the at least two RPTP polypeptides in the absence of the candidate agent. A decrease in the level of binding of the poxvirus polypeptide to the at least two RPTP polypeptides in the presence of the candidate agent indicates that the candidate agent inhibits binding of the poxvirus polypeptide to the at least two RPTP polypeptides In another embodiment, the candidate agent is contacted with a biological sample that comprises LAR, RPTP-σ, and RPTP-δ and the level of binding in the presence and absence of the agent to each of the phosphatases is determined.

Appropriate conditions for permitting interaction of the reaction components according to this method and other methods described herein include, for example, appropriate concentrations of reagents and components (including the poxvirus polypeptide and the candidate agent and the RPTP(s), temperature, and buffers with which a skilled person will be familiar. Concentrations of reaction components, buffers, temperature, and time period sufficient to permit interaction of the reaction components can be determined and/or adjusted according to methods described herein and with which persons skilled in the art are familiar. To practice the methods described herein, a person skilled in the art will also readily appreciate and understand which controls are appropriately included when practicing these methods.

Numerous assays and techniques are practiced by persons skilled in the art for determining the interaction between or binding between a biological molecule and a cognate ligand. Accordingly, interaction between a poxvirus polypeptide, A41L and/or 130L, and any one or more of LAR, RPTP-σ, and RPTP-δ including the effect of a bioactive agent on this interaction and/or binding in the presence of the agent can be readily determined by such assays and techniques, which may include a competitive assay format. Exemplary methods include but are not limited to fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, immunoassays, (such as enzyme-linked immunosorbant assays (ELISA), radioimmunoassay, immunoblotting, immunohistochemistry, and the like), surface plasmon resonance, cell-based assays such as those that use reporter genes, and functional assays (e.g., assays that measure dephosphorylation of a tyrosine phosphorylated substrate by one or more of LAR, RPTP-σ, and RPTP-δ and assays that measure immune function and immunoresponsiveness). Many of the methods described herein and known to those skilled in the art may be adapted to high throughput screening for analyzing large numbers of bioactive agents such as from libraries of compounds to determine the effect of an agent on the binding, interaction, or biological function of the poxvirus polypeptide and/or LAR, RPTP-σ, and RPTP-δ and the effect of an agent on immunoresponsiveness of an immune cell (see, e.g., High Throughput Screening: The Discovery of Bioactive Substances, Devlin, ed., (Marcel Dekker New York, 1997)).

The techniques and assay formats may also include secondary reagents, such as specific antibodies, that are useful for detecting and/or amplifying a signal that indicates formation of a complex, such as between a poxvirus polypeptide (e.g., A41L or 130L) and an RPTP. One or more of the assay components or secondary reagents may be attached to a detectable moiety (or label or reporter molecule) such as an enzyme, cytotoxicity agent, or other reporter molecule, including a dye, radionuclide, luminescent group, fluorescent group, or biotin, or the like. Techniques for radiolabeling of antibodies and other polypeptides are known in the art (see, e.g., Adams, In Vivo 12:11-21 (1998); Hiltunen, Acta Oncol. 32:831-9 (1993)). The detectable moiety may be attached to a polypeptide (e.g., an antibody), such as through any available amino acid side-chain, terminal amino acid, or carbohydrate functional group located in the polypeptide, provided that the attachment or attachment process does not adversely affect the binding properties such that the usefulness of the molecule is abrogated. Particular functional groups include, for example, any free amino, imino, thiol, hydroxyl, carboxyl, or aldehyde group. Attachment of the polypeptide and the detectable moiety may be achieved via such groups and an appropriate functional group in the detectable moiety. The linkage may be direct or indirect through spacing or bridging groups (see, e.g., International Patent Application Publication Nos. WO 93/06231, WO 92/22583, WO 90/091195, and WO 89/01476; see also, e.g., commercial vendors such as Pierce Biotechnology, Rockford, Ill.).

A “biological sample” as used herein refers in certain embodiments to a sample containing at least one of LAR, RPTP-σ, and RPTP-δ or a poxvirus polypeptide or variant thereof. A biological sample may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture (e.g., immune cells, virus infected cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

Candidate agents include but are not limited to an antibody, or antigen-binding fragment thereof, as described herein, and which may be also include a bispecific or bifunctional antibody, chimeric antibody, human or humanized antibody, scFv, or diabody, and the like. Additional agents described herein that are useful for altering the immunoresponsiveness of an immune cell (in certain embodiments, suppressing the immunoresponsiveness of an immune cell) and for treating an immunological disease or disorder include but are not limited to small molecules, peptide-immunoglobulin constant region fusion polypeptides such as a peptide-IgFc fusion polypeptide, aptamers, siRNA polynucleotides, antisense nucleic acids, ribozymes, and peptide nucleic acids.

### Immune Cells and Immune Response

An immune cell is any cell of the immune system, including a lymphocyte and a non-lymphoid cell such as accessory cell. Lymphocytes are cells that specifically recognize and respond to foreign antigens, and accessory cells are those that are not specific for certain antigens but are involved in the cognitive and activation phases of immune responses. For example, mononuclear phagocytes (macrophages), other leukocytes (e.g., granulocytes, including neutrophils, eosinophils, basophils), and dendritic cells function as accessory cells in the induction of an immune response. The activation of lymphocytes by a foreign antigen leads to induction or elicitation of numerous effector mechanisms that function to eliminate the antigen. Accessory cells such as mononuclear phagocytes that effect or are involved with the effector mechanisms are also called effector cells.

Major classes of lymphocytes include B lymphocytes (B cells), T lymphocytes (T cells), and natural killer (NK) cells, which are large granular lymphocytes. B cells are capable of producing antibodies. T lymphocytes are further subdivided into helper T cells (CD4+) and cytolytic or cytotoxic T cells (CD8+). Helper cells secrete cytokines that promote proliferation and differentiation of the T cells and other cells, including B cells and macrophages, and recruit and activate inflammatory leukocytes. Another subgroup of T cells, called regulatory T cells or suppressor T cells actively suppress activation of the immune system and prevent pathological self-reactivity, that is, autoimmune disease. The immunosuppressive cytokines, TGF-beta and interleukin-10 (IL-10), have also been implicated in regulatory T cell function.

In general, an immune response may include a humoral response, in which antibodies specific for antigens are produced by differentiated B lymphocytes known as plasma cells. An immune response may also include, in addition to or instead of a humoral response, a cell-mediated response, in which various types of T lymphocytes act to eliminate antigens by a number of mechanisms. For example, helper T cells that are capable of recognizing specific antigens may respond by releasing soluble mediators such as cytokines to recruit additional cells of the immune system to participate in an immune response. Also, cytotoxic T cells that are also capable of specific antigen recognition may respond by binding to and destroying or damaging an antigen-bearing cell or particle.

An immune response in a host or subject may be determined by any number of well-known immunological methods described herein and with which those having ordinary skill in the art will be readily familiar. Such assays include, but need not be limited to, in vivo or in vitro determination of soluble antibodies, soluble mediators such as cytokines (e.g., IFN-γ, IL-2, IL-4, IL-10, IL-12, and TGF-β), lymphokines, chemokines, hormones, growth factors, and the like, as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

The capability of a poxvirus polypeptide such as A41L or 130L, or a fragment or variant thereof, and of an agent (e.g., an antibody or antigen-binding fragment thereof that specifically binds to LAR, RPTP-σ, and/or RPTP-δ; nucleic acid molecule (such as an aptamer, siRNA, antisense polynucleotide); peptide-IgFc fusion polypeptide) described herein to suppress immunoresponsiveness of an immune cell and thus be useful for treating an immunological disease or disorder, such as an autoimmune disease or inflammatory disease or disorder, cardiovascular disease or disorder, a metabolic disease or disorder, or a proliferative disease or disorder, may be determined and evaluated in any one of a number of animal models described herein and used by persons skilled in the art (see, e.g., reviews by Taneja et al., Nat. Immunol. 2:781-84 (2001); Lam-Tse et al., Springer Semin. Immunopathol. 24:297-321 (2002)). For example, mice that have three genes, Tyro3, Mer, and Axl that encode receptor tyrosine kinases, knocked out exhibit several symptoms of autoimmune diseases, including rheumatoid arthritis and SLE (Lu et al., Science 293:228-29 (2001)). A murine model of spontaneous lupus-like disease has been described using NZB/WF1 hybrid mice (see, e.g., Drake et al., Immunol. Rev. 144:51-74 (1995)). An animal model for type I diabetes that permits testing of agents and molecules that affect onset, modulation, and/or protection of the animal from disease uses MHC transgenic (Tg) mice. Mice that express the HLA-DQ8 transgene (HLA-DQ8 is the predominant predisposing gene in human type 1 diabetes) and the HLA-DQ6 transgene (which is diabetes protective) were crossed with RIP(rat insulin promoter).B7-1-Tg mice to provide HLA-DQ8 RIP.B7-1 transgenic mice that develop spontaneous diabetes (see Wakeland et al., Curr. Opin. Immunol 11:701-707 (1999); Wen et al., J. Exp. Med. 191:97-104 (2000)). (See also Brondum et al., Horm. Metab. Res. 37 Suppl 1:56-60 (2005)).

Animal models that may be used for characterizing agents that are useful for treating rheumatoid arthritis include a collagen-induced arthritis model (see, e.g., Kakimoto, Chin. Med. Sci. J. 6:78-83 (1991); Myers et al., Life Sci. 61:1861-78 (1997)) and an anti-collagen antibody-induced arthritis model (see, e.g., Kakimoto, supra). Other applicable animal models for immunological diseases include an experimental autoimmune encephalomyelitis model (also called experimental allergic encephalomyelitis model), an animal model of multiple sclerosis; a psoriasis model that uses AGR129 mice that are deficient in type I and type II interferon receptors and deficient for the recombination activating gene 2 (Zenz et al., Nature 437:369-75 (2005); Boyman et al., J. Exp. Med. 199:731-36 (2004); published online Feb. 23, 2004); and a TNBS (2,4,6-trinitrobenzene sulphonic acid) mouse model for inflammatory bowel disease. Numerous animal models for cardiovascular disease are available and include models described in van Vlijmen et al., J Clin. Invest. 93:1403-10 (1994); Kiriazis et al., Annu. Rev. Physiol. 62:321-51 (2000); Babu et al., Methods Mol. Med. 112:365-77 (2005).

### Treatment of Immunological Disorders and Disease

In another embodiment, methods are provided for treating and/or preventing immunological diseases and disorders, particularly an inflammatory disease or disorder, an autoimmune disease or disorder, cardiovascular disease or disorder, a metabolic disease or disorder, or a proliferative disease or disorder disease as described herein. A subject in need of such treatment may be a human or may be a non-human primate or other animal (i.e., veterinary use) who has developed symptoms of an immunological disease or who is at risk for developing an immunological disease. Examples of non-human primates and other animals include but are not limited to farm animals, pets, and zoo animals (e.g., horses, cows, buffalo, llamas, goats, rabbits, cats, dogs, chimpanzees, orangutans, gorillas, monkeys, elephants, bears, large cats, etc.). In certain embodiments, compositions are provided that comprise an antibody, or antigen-binding fragment thereof, bispecific antibody, fusion polypeptide, RPTP Ig domain polypeptide (monomer or multimer), macromolecule, nucleic acid, or other agent, as described herein plus a pharmaceutically acceptable excipient.

As described herein, a method is provided for altering (e.g., suppressing or enhancing) an immune response in a subject (host or patient) who has or who is at risk for developing an immunological disease or disorder, by administering a composition that comprises a pharmaceutically acceptable carrier and an antibody, or antigen-binding fragment thereof, that specifically binds to at least one of LAR, RPTP-σ, and RPTP-δ. In particular embodiments, the antibody or antigen-binding fragment thereof is capable of inhibiting, preventing, or competing with binding of A41L or 130L to the RPTP. In certain embodiments, the composition comprises an antibody, or antigen-binding fragment thereof, that specifically binds to RPTP-σ, and in another certain embodiment, the composition comprises an antibody, or antigen-binding fragment thereof, that specifically binds to RPTP-δ. Also provided is a method for altering (e.g., suppressing or enhancing) an immune response in a subject (host or patient) who has or who is at risk for developing an immunological disease or disorder, by administering a composition that comprises a pharmaceutically acceptable carrier and an antibody (i.e., at least) or antigen-binding fragment thereof, that specifically binds to at least two of LAR, RPTP-σ, and RPTP-δ (e.g., LAR and RPTP-σ; LAR and RPTP-δ; RPTP-σ and RPTP-δ). In a particular embodiment, such a method suppresses an immune response in a subject. Alternatively, the composition comprises an antibody, or antigen-binding fragment thereof, that specifically binds to all three RPTPs. In certain embodiments, the composition comprises a pharmaceutically acceptable carrier and at least one antibody that binds to all three of LAR, RPTP-σ, and RPTP-δ. In other embodiments, the composition comprises any two or more of the antibodies, or antigen-binding fragment thereof, described herein. Accordingly, a composition for altering (suppressing or enhancing) an immune response comprises at least one antibody that binds to LAR, at least one antibody that binds to RPTP-σ, and at least one antibody that binds to RPTP-δ. In another embodiment, the composition comprises at least one antibody that binds to LAR, and at least one antibody that binds to both RPTP-σ and RPTP-δ. Also contemplated and described herein is a composition that comprises at least one first antibody that binds any two of LAR, RPTP-σ, and RPTP-δ and at least one second antibody that binds to the RPTP that is not specifically recognized by the at least one first antibody.

In another embodiment, a method for treating an immunological disease or disorder is provided wherein the method comprises administering to a subject in need thereof a pharmaceutically suitable carrier and an agent that alters a biological activity of at least one of LAR, RPTP-σ, or RPTP-δ, or that alters a biological activity of at least two of or all three of LAR, RPTP-σ, and RPTP-δ. An agent as described herein (including an antibody, or antigen-binding fragment thereof; a small molecule; an aptamer; an antisense polynucleotide; a small interfering RNA (siRNA); a peptide-IgFc fusion polypeptide or peptide Ig constant region domain fusion polypeptide; a RPTP Ig-like domain polypeptide (monomer or multimer), and a RPTP Ig-like domain-Ig constant region domain fusion polypeptide, all of which are described in detail herein) that is useful for treating an immunological disease or disorder is capable of altering (increasing or decreasing in a statistically significant or biological significant manner) at least one biological activity (function) of the at least one RPTP. In other embodiments, the agent alters at least one biological function of at least one, two or all three of LAR, RPTP-σ, and RPTP-δ. As described herein, these protein tyrosine phosphatases dephosphorylate tyrosyl phosphoproteins, and along with protein tyrosine kinases regulate reversible tyrosine phosphorylation in a dynamic relationship that is integrated in a cell. The regulated phosphorylation and dephosphorylation of tyrosine residues of substrates in signal transduction pathways is a major control mechanism for cellular processes such as cell growth, cell proliferation, metabolism, differentiation, and locomotion. An agent used for treating an immunological disease or disorder may therefore affect or alter any one or more of the biological activities or functions of at least one, two, or all three of LAR, RPTP-σ, and RPTP-δ including (1) the capability to dephosphorylate a tyrosyl phosphorylated substrate (i.e., affect the catalytic activity); (2) the capability to affect cell proliferation; (3) the capability to affect cellular metabolism; (4) the capability to affect cell differentiation; and (5) the capability to affect cell locomotion; (6) the capability to affect the function of another component in the same signal transduction pathway.

The agents, compositions, antibodies or fragments thereof, fusion polypeptides, RPTP Ig domain polypeptides, molecules, and methods described herein may be used for treating (i.e., curing, preventing, ameliorating the symptoms of, or slowing, inhibiting, or stopping the progression of) an immunological disease or disorder. A particular disease or disorder may be treated by administering an effective amount of a particular agent, which can be readily determined by persons skilled in the medical art. Such diseases and disorders that are autoimmune or inflammatory disorders include but are not limited to multiple sclerosis, rheumatoid arthritis, systemic lupus erythematosus (SLE), graft versus host disease (GVHD), sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. An immunological disorder or disease also includes a cardiovascular disease or disorder, a metabolic disease or disorder, or a proliferative disease or disorder. A cardiovascular disease or disorder that may be treated according to the methods and with the agents described herein includes, for example, atherosclerosis, endocarditis, hypertension, or peripheral ischemic disease. Metabolic diseases that also are immunological disorders or diseases include diabetes, Crohn's Disease, and inflammatory bowel disease. An exemplary proliferative disease is cancer.

As used herein, a patient (or subject) may be any mammal, including a human, that may have or be afflicted with an immunological disease or disorder, or that may be free of detectable disease. Accordingly, the treatment may be administered to a subject who has an existing disease, or the treatment may be prophylactic, administered to a subject who is at risk for developing the disease or condition.

A pharmaceutical composition may be a sterile aqueous or non-aqueous solution, suspension or emulsion, which additionally comprises a physiologically acceptable excipient (pharmaceutically acceptable or suitable excipient or carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient or carrier known to those of ordinary skill in the art for use in pharmaceutical compositions may be employed in the compositions described herein. Excipients for therapeutic use are well known, and are described, for example, in Remingtons Pharmaceutical Sciences, Mack Publishing Co. (A. R. Gennaro ed. 1985). In general, the type of excipient is selected based on the mode of administration. Pharmaceutical compositions may be formulated for any appropriate manner of administration, including, for example, topical, oral, nasal, intrathecal, rectal, vaginal, intraocular, subconjunctival, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The agents described herein, including antibodies and antigen-binding fragments thereof, and bispecific antibody that specifically bind to at least one of LAR, PTP-σ, and RPTP-δ, small molecules, nucleic acid molecules, RPTP Ig-like domain polypeptides, and peptide and polypeptide fusion proteins, may be formulated for sustained or slow release. Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

The dose of the composition for treating an immunological disease or disorder may be determined according to parameters understood by a person skilled in the medical art. Accordingly, the appropriate dose may depend upon the patient's (e.g., human) condition, that is, stage of the disease, general health status, as well as age, gender, and weight, and other factors familiar to a person skilled in the medical art.

Pharmaceutical compositions may be administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with an immunological disease or disorder.

Optimal doses may generally be determined using experimental models and/or clinical trials. The optimal dose may depend upon the body mass, weight, or blood volume of the patient. In general, the amount of polypeptide, such as an antibody or antigen-binding fragment thereof, or a fusion polypeptide, or RPTP Ig domain polypeptide as described herein, present in a dose, or produced in situ by DNA present in a dose, ranges from about 0.01 μg to about 1000 μg per kg of host. The use of the minimum dosage that is sufficient to provide effective therapy is usually preferred. Patients may generally be monitored for therapeutic or prophylactic effectiveness using assays suitable for the condition being treated or prevented, which assays will be familiar to those having ordinary skill in the art. Suitable dose sizes will vary with the size of the patient, but will typically range from about 1 ml to about 500 ml for a 10-60 kg subject.

For pharmaceutical compositions comprising an agent that is a nucleic acid molecule including an aptamer, siRNA, antisense, or ribozyme, or peptide-nucleic acid, the nucleic acid molecule may be present within any of a variety of delivery systems known to those of ordinary skill in the art, including nucleic acid, and bacterial, viral and mammalian expression systems such as, for example, recombinant expression constructs as provided herein. Techniques for incorporating DNA into such expression systems are well known to those of ordinary skill in the art. The DNA may also be “naked,” as described, for example, in Ulmer et al., Science 259:1745-49, 1993 and reviewed by Cohen, Science 259:1691-1692, 1993. The uptake of naked DNA may be increased by coating the DNA onto biodegradable beads, which are efficiently transported into the cells.

Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland and Huang, Handb. Exp. Pharmacol. 137:165-92 (1999); Lee et al., ACS Symp. Ser. 752:184-92 (2000); U.S. Pat. No. 6,395,713; International Patent Application Publication No. WO 94/02595); Selbo et al., Int. J. Cancer 87:853-59 (2000); Selbo et al., Tumour Biol. 23:103-12 (2002); U.S. Patent Application Publication Nos. 2001/0007666, and 2003/077829). Such delivery methods known to persons having skill in the art, include, but are not restricted to, encapsulation in liposomes, by iontophoresis, or by incorporation into other vehicles, such as biodegradable polymers; hydrogels; cyclodextrins (see, e.g., Gonzalez et al., Bioconjug. Chem. 10:1068-74 (1999); Wang et al., International Application Publication Nos. WO 03/47518 and WO 03/46185); poly(lactic-co-glycolic)acid (PLGA) and PLCA microspheres (also useful for delivery of peptides and polypeptides and other substances) (see, e.g., U.S. Pat. No. 6,447,796; U.S. Patent Application Publication No. 2002/130430); biodegradable nanocapsules; and bioadhesive microspheres, or by proteinaceous vectors (International Application Publication No. WO 00/53722). In another embodiment, the nucleic acid molecules for use in altering (suppressing or enhancing) an immune response in an immune cell and for treating an immunological disease or disorder can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives (see also, e.g., U.S. Patent Application Publication No. 2003/0077829).

Also provided herein are methods of manufacture for producing an agent that alters (suppresses or enhances) immunoresponsiveness of an immune cell and that is useful for treating a subject who has or who is at risk of developing an immunological disease or disorder. In one embodiment, such a method of manufacture comprises (a) identifying an agent that suppresses immunoresponsiveness of an immune cell according to methods described herein and practiced in the art. For example, identifying an agent comprises contacting (i) a candidate agent; (ii) an immune cell that expresses at least one receptor-like protein tyrosine phosphatase (RPTP) polypeptide selected from leukocyte common antigen-related protein (LAR); RPTP-σ; and RPTP-δ; and (iii) A41L, under conditions and for a time sufficient to permit interaction between the at least one RPTP polypeptide and a poxvirus polypeptide, such as A41L and 130L. Then binding of the poxvirus polypeptide to the immune cell in the presence of the candidate agent is determined and compared to binding of the poxvirus polypeptide to the immune cell in the absence of the candidate agent, wherein a decrease in binding of the poxvirus polypeptide to the immune cell in the presence of the candidate agent indicates that the candidate agent suppresses immunoresponsiveness of the immune cell. The agent is then produced according to methods known in the art for producing the agent.

The agent may be any agent described herein, such as, for example, an antibody, or antigen-binding fragment thereof; bispecific antibody, a small molecule; an aptamer; an antisense polynucleotide; a small interfering RNA (siRNA); RPTP Ig-like domain polypeptide (monomer or multimer) and a peptide-IgFc fusion polypeptide. In a particular embodiment, the agent is an antibody, or antigen-binding fragment thereof, which may be produced according to methods described herein and that are adapted for large-scale manufacture. For example, production methods include batch cell culture, which is monitored and controlled to maintain appropriate culture conditions. Purification of the antibody, or antigen-binding fragment thereof, may be performed according to methods described herein and known in the art and that comport with guidelines of domestic and foreign regulatory agencies.

The following Examples are offered for the purpose of illustrating the present invention and are not to be construed to limit the scope of this invention.

### EXAMPLES

### Example 1

### Identification of RPTPs Expressed on Immune Cells that Bind A41L

This Example describes a method for identifying cell surface polypeptides that bind to A41L.

A recombinant expression vector comprising a polynucleotide that encoded a Cowpox A41L fusion polypeptide was constructed for a tandem affinity purification (TAP) procedure (also called TAP tag procedure) (see also, e.g., Rigaut et al. Nat. Biotech. 17:1030-32 (1999); Puig et al., Methods 24:218-29 (2001); Knuesel et al. Mol. Cell. Proteomics 2:1225-33 (2003)). The construct called A41LCRFC was prepared and the fusion polypeptide expressed and isolated according to standard molecular biology and affinity purification techniques and methods. A schematic of the construct is provided in FIG. 2. The A41LCRFC construct included a nucleotide sequence that encoded a mature A41L coding sequence from Cowpox virus fused to the C-terminus of the human growth hormone leader peptide. The CRFC tandem affinity tag was fused to the C-terminus of A41L. The CRFC tag included a human influenza virus hemagglutinin peptide, the HA epitope, amino acids YPYDVDYA (SEQ ID NO:67), for which antibodies are commercially available, permitting detection of the expression fusion polypeptide by immunochemistry methods, such as fluorescence activated cell sorting (FACS) or immunoblotting. Fused to the carboxyl terminal end of the HA epitope was a Protein C-tag, amino acids EDQVDPRLIDGK (SEQ ID NO:68), which is derived form the heavy chain of human Protein C. To the carboxyl end of the Protein C-tag was fused a Human Rhinovirus HRV3C protease site, amino acids LEVLFQGP (SEQ ID NO:69); and to the carboxyl end of the HRV3C protease site was fused a mutein derivative of the Fc portion of a human IgG.

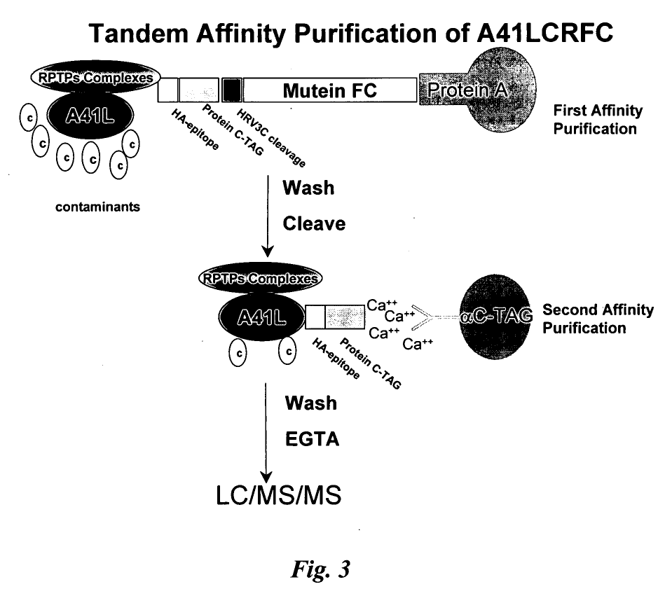

A schematic illustrating the TAP tag procedure is presented in FIG. 3. Ten μg of the A41 LCRFC fusion polypeptide that was bound to Protein A was incubated with cell lysates prepared from 5×10⁶ monocytes. A variety of normal cells and tumor cell types may be used to identify cellular polypeptides that bind to or interact with A41L, including B cells and T cells (activated or non-activated), macrophages, epithelial cells, fibroblasts, and cell lines such as Raji (B cell lymphoma), THP-1 (acute monocytic leukemia), and Jurkat (T cell leukemia).

The A41LCRFC/cell lysate complexes were washed and then subjected to cleavage by the HRV3C protease, which released A41L and associated proteins. Calcium chloride (1 M) was added to the released A41L/cell lysate complexes, which were then applied to an anti-protein C-Tag affinity resin. Calcium chloride is required for the interaction of anti-C-tag and the C-tag epitope. The complexes bound to the anti-protein C-Tag affinity resin were washed in a buffer containing calcium chloride and then eluted by calcium chelation using EGTA. The subsequent eluent was digested with trypsin and the digested A41l complexes were subjected to direct tandem mass spectrometry to identify A41L and its associated proteins.

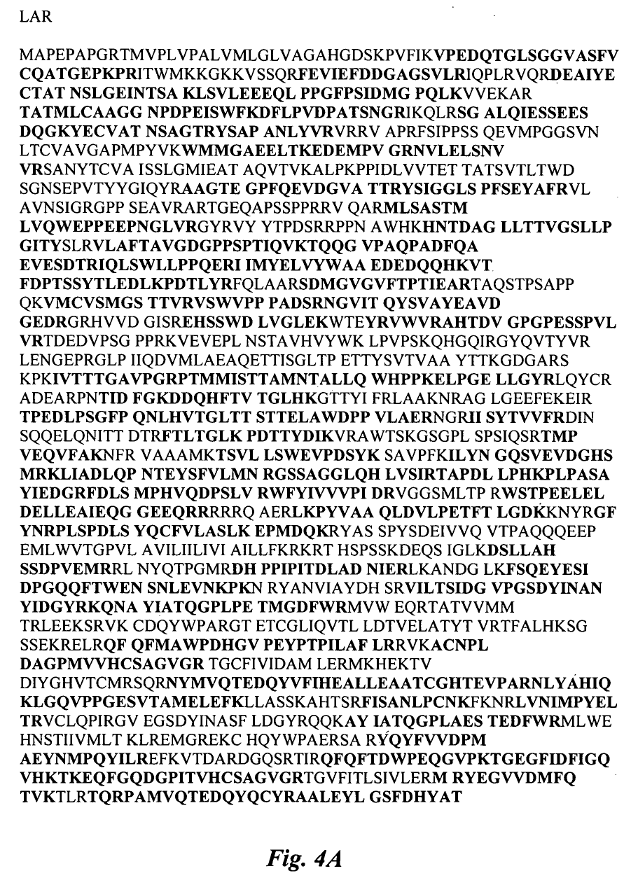

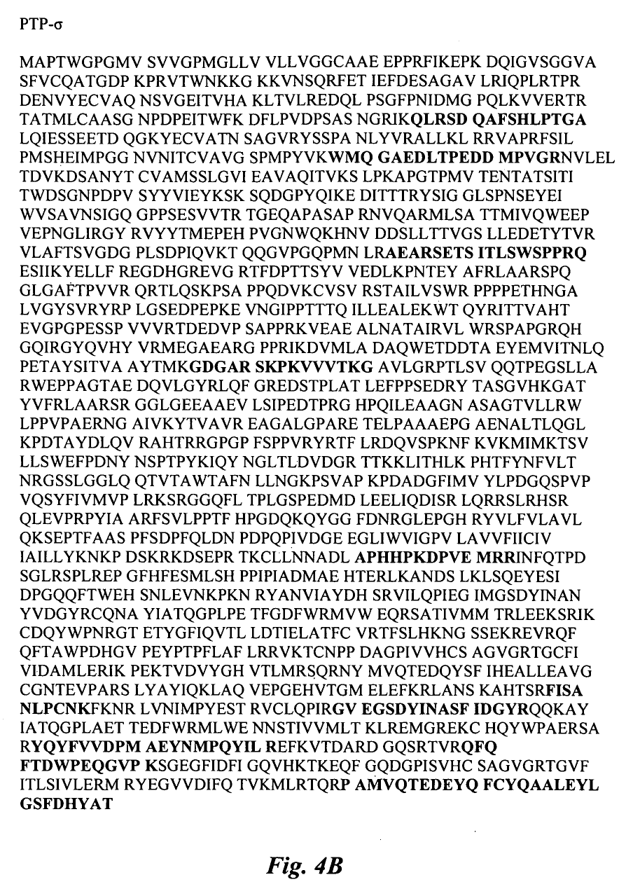

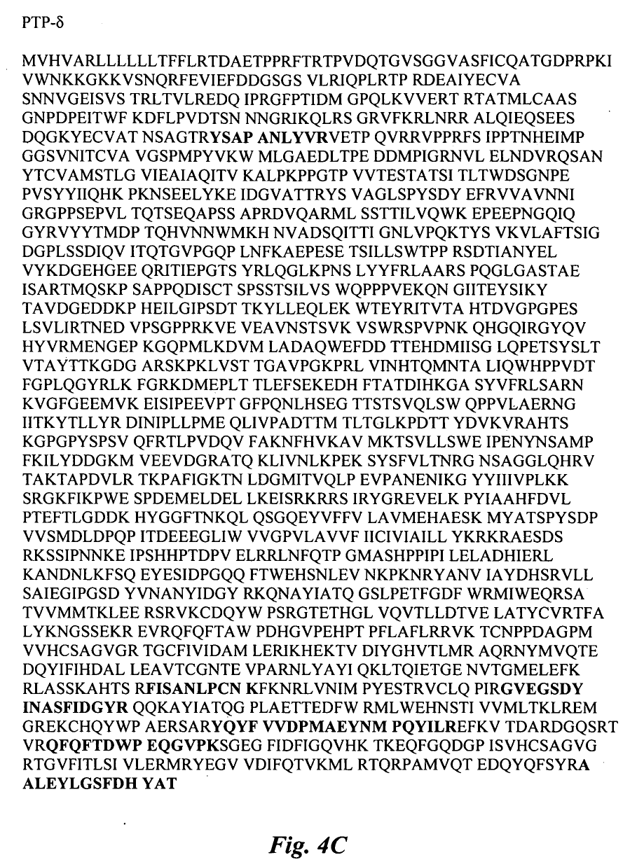

The sequences of the trypsin-generated peptides were identified by mass spectrometry. The peptides were identified as portions of the receptor-like protein tyrosine phosphatases, LAR, RPTP-σ, and RPTP-δ as shown in FIGS. 4A, 4B, and 4C, respectively.

### Example 2

### Preparation of A41L-Fc Fusion Polypeptides

This example describes preparation of recombinant expression vectors for expression of an A41L-Fc fusion polypeptide and an A41L-mutein Fc fusion polypeptide.

Recombinant expression vectors were prepared according to methods routinely practiced by a person skilled in the molecular biology art. A polynucleotide encoding A41L-Fc and a polynucleotide encoding A41L-mutein Fc were cloned into the multiple cloning site of the vector, pDC409 (see, e.g., U.S. Pat. No. 6,512,095 and U.S. Pat. No. 6,680,840, and references cited therein). The amino acid sequence of the A41L-Fc polypeptide is set forth in SEQ ID NO:74, and the amino acid sequence of the A41L-mutein Fc polypeptide is set forth in SEQ ID NO: 73 (see FIG. 5). The nucleotide sequence that encodes the mutein Fc (human IgG1) polypeptide (SEQ ID NO:77) is set forth in SEQ ID NO:78. Ten to twenty micrograms of each expression plasmid were transfected into HEK293T cells or COS-7 cells (American Type Tissue Collection (ATCC), Manassas, Va.) that were grown in 10 cm diameter standard tissue culture plates to approximately 80% confluency. Transfection was performed using Lipofectamine™ Plus™ (Invitrogen Corp., Carlsbad, Calif.). The transfected cells were cultured for 48 hours, and then supernatant from the cell cultures was harvested. The A41L fusion proteins were purified by Protein A sepharose affinity chromatography according to standard procedures.

### Example 3

### Identification of RPTPs Expressed on Immune Cells that Bind Yaba-like Disease Virus 130L

This Example describes a method for identifying cell surface polypeptides that bind to 130L.

A recombinant expression vector comprising a polynucleotide that encoded A recombinant expression vector comprising a polynucleotide that encodes a 130L fusion polypeptide was constructed for a tandem affinity purification (TAP) procedure (also called TAP tag procedure) as described in Example 1. The construct was prepared and the fusion polypeptide expressed and isolated according to standard molecular biology and affinity purification techniques and methods.

The 130L tandem affinity tag construct included a nucleotide sequence that encodes a mature 130L amino acid sequence from YLDV, which was fused to a nucleotide sequence that encodes the C-terminus of the human growth hormone signal peptide amino acid sequence (MATGSRTSLLLAFGLLCLPWLQEGSA (SEQ ID NO:153) (i.e., the 5′ end of the nucleotide sequence encoding 130L is fused to the 3′ end of the nucleotide sequence encoding the signal peptide).

The tandem affinity tag was fused to the C-terminus of 130L. The tag included a human influenza virus hemagglutinin peptide, the HA epitope, amino acids YPYDVDYA (SEQ ID NO:141), for which antibodies are commercially available, permitting detection of the expression fusion polypeptide by immunochemistry methods, such as fluorescence activated cell sorting (FACS) or immunoblotting. Fused to the carboxyl terminal end of the HA epitope was a Protein C-tag, amino acids EDQVDPRLIDGK (SEQ ID NO:142), which is derived from the heavy chain of human Protein C. To the carboxyl end of the Protein C-tag was fused a Human Rhinovirus HRV3C protease site, amino acids LEVLFQGP (SEQ ID NO:143); and to the carboxyl end of the HRV3C protease site is fused a mutein derivative of the Fc portion of a human IgG (e.g., SEQ ID NO:146).

Ten μg of the recombinantly expressed 130L fusion polypeptide was permitted to bind to a Protein A affinity matrix. The 130L fusion polypeptide that was bound to Protein A was incubated with cell lysates prepared from 5×10⁶ monocytes. A variety of normal cells and tumor cell types may be used to identify cellular polypeptides that bind to or interact with 130L, including B cells and T cells (activated or non-activated), macrophages, epithelial cells, fibroblasts, and cell lines such as Raji (B cell lymphoma), THP-1 (acute monocytic leukemia), and Jurkat (T cell leukemia).

The 130L fusion polypeptide/cell lysate complexes were washed and then subjected to cleavage by the HRV3C protease, which releases 130L and associated proteins. Calcium chloride (1 M) was added to the released 130L/cell lysate complexes, which were then applied to an anti-protein C-Tag affinity resin. Calcium chloride is required for the interaction of anti-C-tag and the C-tag epitope. The complexes that bind to the anti-protein C-Tag affinity resin were washed in a buffer containing calcium chloride and then eluted by calcium chelation using EGTA. The subsequent eluent was digested with trypsin and the digested 130L complexes were subjected to direct tandem mass spectrometry to identify 130L and its associated proteins.

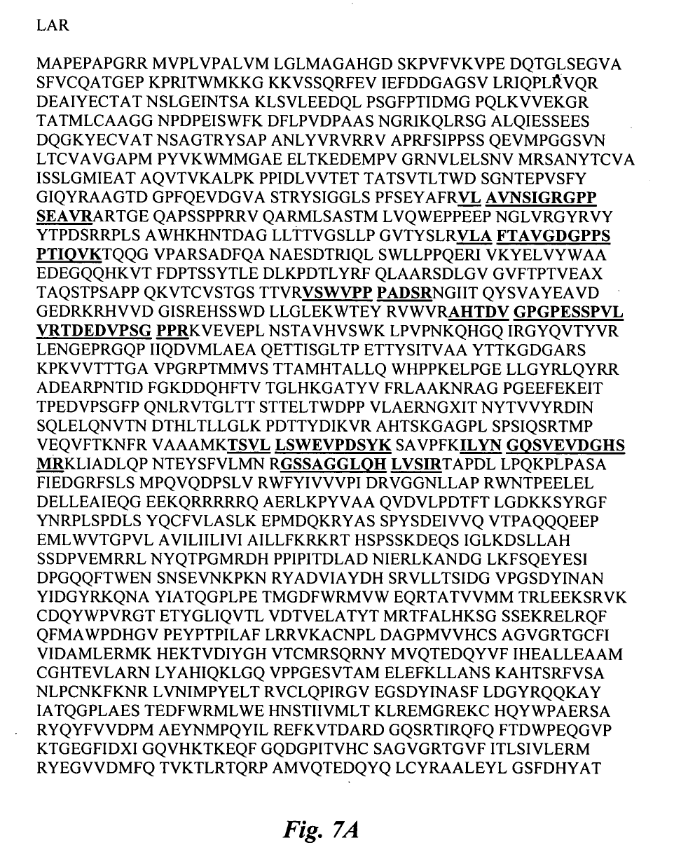

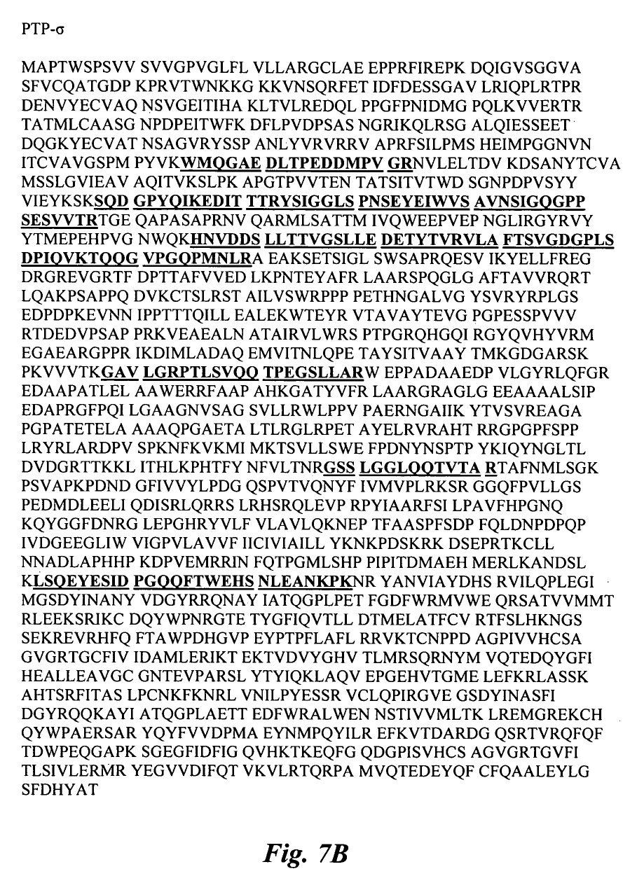

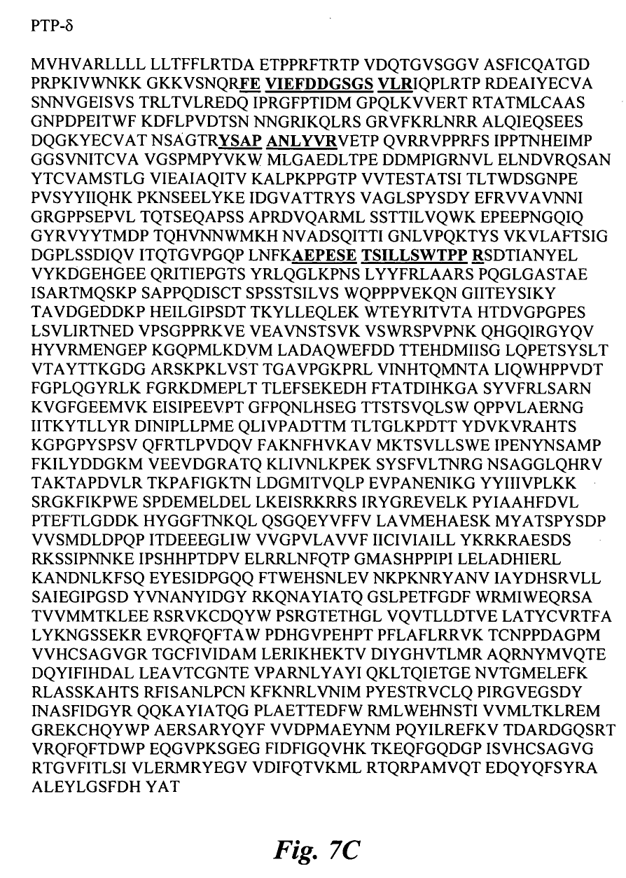

The sequences of the trypsin-generated peptides were identified by mass spectrometry. The peptides were identified as portions of the receptor-like protein tyrosine phosphatases, LAR, RPTP-σ, and RPTP-δ as shown in FIGS. 7A, 7B, and 7C, respectively.

### Example 4

### Induction of IFN-Gamma in Non-Adherent PBMCs by an LAR (Ig Domains)-FC Fusion Protein

This Example describes production of IFN-γ in peripheral blood mononuclear cells (PBMCs) in the presence and absence of heterologous donor cells.

A recombinant expression vector for expression of the LAR-Fc fusion protein was prepared according to methods routinely practiced by a person skilled in the molecular biology art. A nucleotide sequence encoding the first immunoglobulin-like domain (Ig-1), the second immunoglobulin-like domain (Ig-2), and the third immunoglobulin-like domain (Ig-3) of LAR was fused in frame to a nucleotide sequence that encoded an Fc mutein polypeptide. The Fc mutein polypeptide was derived from a human IgG1 immunoglobulin. The expression construct was transfected into cells and the expressed fusion polypeptide was isolated from the cell supernatants by Protein A affinity chromatography.

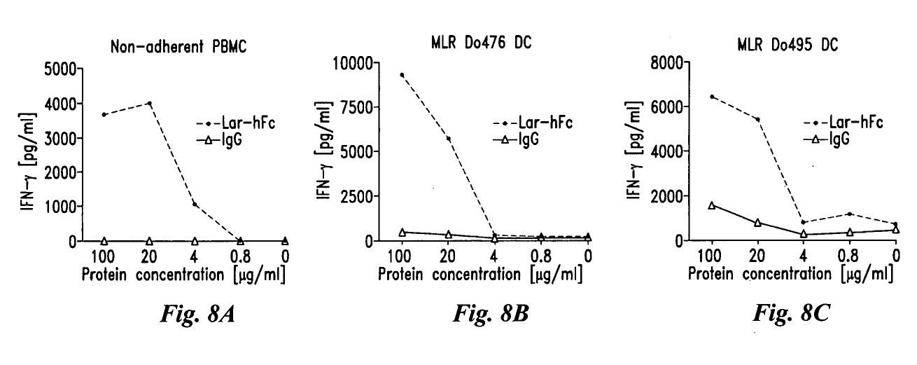

Human PBMCs were isolated from freshly drawn whole blood according to standard methods in the art. The PBMCs were enriched for non-adherent PBMC by placing the PBMCs in a tissue culture flask in RPMI containing 2% human serum for 2 hours and then gently removing the cell culture supernatant containing the nonadherent cells. The non-adherent cells (2×10⁵) were then cultured alone or in a mixed lymphocyte reaction with 10⁴ monocyte-derived dendritic cells from each of two heterologous donors (Do476 and Do495) at 0.8, 4, 20, and 100 μg/ml LAR-Fc or human IgG. After 18 hours, IFN-γ production by the non-adherent PBMC was determined by measuring. The concentration of IFN-γ in the cell supernatants was determined by ELISA (DuoSet ELISA Human IFN-γ, Cat. No. D6285, R & D Systems, Minneapolis, Minn.). As shown in FIG. 8, the LAR-Fc fusion protein enhanced the secretion of IFN-γ by non-adherent PBMC in the mixed lymphocyte reaction (FIGS. 8B and 8C). In addition, the non-adherent PBMC treated with LAR-Fc produced IFN-γ in the absence of an antigenic stimuli (FIG. 8A).

### Example 5

### Gel Filtration Chromatography of LAR (Ig Domains)-Fc Fusion Protein

This Example describes size exclusion chromatograph of the LAR Ig1-Ig2-Ig3-Fc (LAR-Fc) fusion polypeptide.

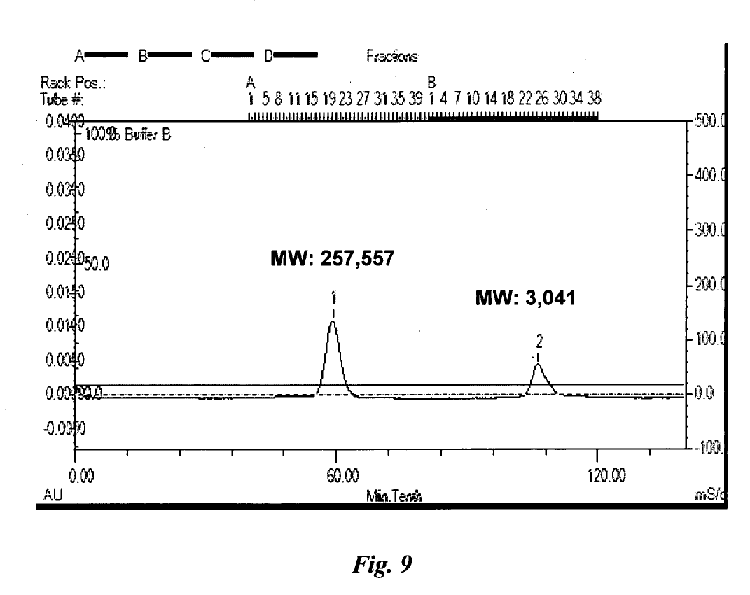

The LAR-Fc fusion polypeptide was prepared as described in Example 4. The fusion polypeptide was then analyzed by HPLC using a gel filtration column to obtain an estimated molecular weight of the fusion polypeptide. The elution profile is presented in FIG. 9. The apparent molecular weight of the polypeptide was determined by comparing the time of elution (minutes) with elution times of standardized molecular weight marker polypeptides. The estimated molecular weight according to the gel filtration method was approximately 260,000 Daltons. The LAR-Fc fusion polypeptide is expected to form a dimer by virtue of the interaction between two Fc polypeptides, and the calculated molecular weight of is 140,000 Daltons. These data suggest that the Stoke's radius of the fusion polypeptide is greater than predicted if the fusion polypeptide dimer had a globular structure. Without wishing to be bound by theory, Ig domains of each of two of the LAR Fc fusion polypeptides may interact with each other to form a dimeric structure, independent and different from the interaction between the Fc portions of two fusion polypeptides.

### Example 6

### Interaction Between A41L and LAR Ig Domains

This Example describes interaction between A41L and the immunoglobulin-like domains of LAR.

Recombinant expression vectors for expression of LAR-Fc fusion polypeptides were prepared using standard molecular biology techniques and as described in Example 2. The fusion polypeptides included TAP-Fc fusion polypeptides: a fusion polypeptide with the first, second, and third immunoglobulin-like domains with TAP sequences, which included a human IgG Fc polypeptide sequence (LAR Ig1-2-3-tapFC); a fusion polypeptide of the first immunoglobulin-like domain of LAR fused to TAP-Fc (LAR Ig1-tapFC); and a fusion polypeptide of the first and second immunoglobulin-like domains fused to TAP-Fc (LAR Ig1-Ig2-tapFC). The TAP constructs were expressed in 293-T17 cells. Cells that were transfected with this expression vector encoding LAR Ig1-Ig2-tapFC did not express the fusion polypeptide. Also included was a purified LAR Ig1-Ig2-Ig3-Fc fusion polypeptide and a P35-FC polypeptide (non-RPTP, non-A41L polypeptide control).

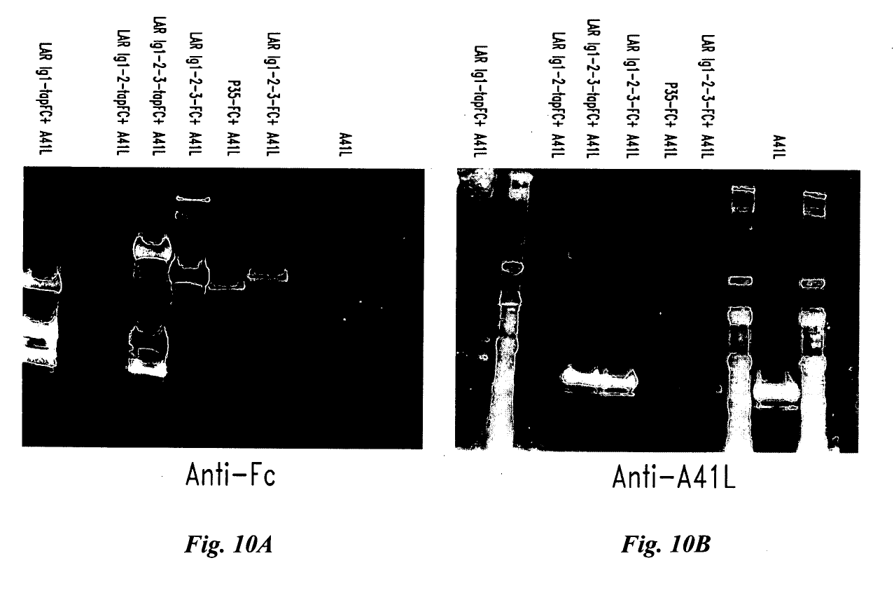

Immunoprecipitation reactions were performed. Cells were transfected with recombinant expression constructs encoding each of the TAP-Fc fusion polypeptides described above, cultured, and the cell supernatants collected. The supernatants were combined with purified A41L polypeptide (monomer) to which protein A conjugated beads were added. The P35-FC and LAR Ig1-Ig2-Ig3-Fc fusion polypeptide, included as controls, were purified polypeptides and incubated with purified A41L. Then the fusion polypeptides were isolated from the immunoprecipitates and subjected to SDS-PAGE. The presence of A41L bound to the LAR fusion polypeptides was analyzed by immunoblotting. The results are presented in FIG. 10. A41L bound to the LAR fusion polypeptides that included all three immunoglobulin-like domains but did not bind to the LAR Ig1-tapFC fusion polypeptide.

From the foregoing it will be appreciated that, although specific embodiments of the invention have been described herein for purposes of illustration, various modifications may be made without deviating from the spirit and scope of the invention. Those skilled in the art will recognize, or be able to ascertain, using no more than routine experimentation, many equivalents to the specific embodiments of the invention described herein. Such equivalents are intended to be encompassed by the following claims.

We claim the following:

1. An isolated antibody, or antigen-binding fragment thereof, (a) that specifically binds to at least two receptor-like protein tyrosine phosphatase (RPTP) polypeptides selected from (i) leukocyte common antigen-related protein (LAR); (ii) RPTP-σ; and (iii) RPTP-δ; and (b) that competitively inhibits binding of a poxvirus polypeptide to the at least two RPTP polypeptides.

2. An isolated antibody, or antigen-binding fragment thereof, that specifically binds to at least one receptor-like protein tyrosine phosphatase (RPTP) present on the cell surface of an immune cell, wherein the at least one RPTP is RPTP-σ or RPTP-δ, and wherein binding of the antibody, or antigen-binding fragment thereof, to the RPTP that is present on the cell surface of the immune cell suppresses immunoresponsiveness of the immune cell.

3. The antibody according to either claim 1 or 2, wherein the antibody is a polyclonal antibody or a monoclonal antibody.

4. The antigen-binding fragment according to either claim 1 or 2, wherein the antigen-binding fragment is selected from F(ab′)₂, Fab′, Fab, Fd, Fv, and single chain Fv (scFv).

5. The antibody according to either claim 1 or claim 2 wherein the poxvirus polypeptide is either A41L or Yaba-like Disease Virus 130L.

6. A bispecific antibody comprising (a) a first antigen-binding moiety that is capable of specifically binding to a receptor-like protein tyrosine phosphatase (RPTP), wherein the RPTP is selected from (i) leukocyte common antigen-related protein (LAR); (ii) RPTP-σ; and (iii) RPTP-δ; and (b) a second antigen-binding moiety that is capable of specifically binding to a RPTP, wherein the RPTP is selected from (i) leukocyte common antigen-related protein (LAR); (ii) RPTP-σ; and (iii) RPTP-δ, wherein the first antigen-binding moiety and the second antigen-binding moiety are different, and wherein the bispecific antibody suppresses immunoresponsiveness of an immune cell.

7. A fusion polypeptide comprising (a) an immunoglobulin-like domain 2 polypeptide of a first receptor-like protein tyrosine phosphatase (RPTP); (b) an immunoglobulin-like domain 3 polypeptide of a second RPTP; and (c) an immunoglobulin Fc polypeptide or mutein thereof, wherein each of the first RPTP and the second RPTP is selected from (i) leukocyte common antigen-related protein (LAR); (ii) RPTP-σ; and (iii) RPTP-δ, and wherein the first and second RPTP are the same or different.

8. The fusion polypeptide of claim 7 wherein the first RPTP and the second RPTP are the same.

9. The fusion polypeptide of claim 7 wherein the first RPTP is RPTP-σ and the second RPTP is RPTP-σ, and wherein the fusion polypeptide further comprises an immunoglobulin-like domain 1 polypeptide of RPTP-σ; or wherein the first RPTP is RPTP-δ and the second RPTP is RPTP-δ, and wherein the fusion polypeptide further comprises an immunoglobulin-like domain 1 polypeptide of RPTP-δ.

10. A composition comprising (a) at least one immunoglobulin-like domain 2 polypeptide of a first receptor-like protein tyrosine phosphatase (RPTP) and (b) at least one immunoglobulin-like domain 3 polypeptide of a second RPTP, wherein the first and second RPTP are the same or different and selected from (i) leukocyte common antigen-related protein (LAR); (ii) RPTP-σ; and (iii) RPTP-δ.

11. The composition of claim 10 wherein the first RPTP and the second RPTP are the same.

12. The composition of claim 10 wherein the first RPTP is RPTP-σ and the second RPTP is RPTP-σ, and wherein the composition further comprises an immunoglobulin-like domain 1 polypeptide of RPTP-σ; or wherein the first RPTP is RPTP-δ and the second RPTP is RPTP-δ, and wherein the composition further comprises an immunoglobulin-like domain 1 polypeptide of RPTP-δ.

13. A composition comprising a polypeptide dimer wherein the dimer comprises (a) a first monomer comprising an immunoglobulin-like domain 2 polypeptide and an immunoglobulin-like domain 3 polypeptide of a first receptor-like protein tyrosine phosphatase (RPTP); and (b) a second monomer comprising an immunoglobulin-like domain 2 polypeptide and an immunoglobulin-like domain 3 polypeptide of a second RPTP, wherein the first and second RPTP are the same or different and selected from (i) leukocyte common antigen-related protein (LAR); (ii) RPTP-σ; and (iii) RPTP-δ.

14. The composition of claim 13 wherein the first RPTP and the second RPTP are different.

15. The composition of claim 13 wherein the first RPTP and the second RPTP are the same.

16. The composition of claim 13 wherein the first monomer further comprises an immunoglobulin-like domain 1 of the first RPTP, and wherein the second monomer further comprises an immunoglobulin-like domain 1 of the second RPTP.

17. The composition according to claim 13 wherein the first monomer is fused to an immunoglobulin Fc polypeptide, and wherein the second monomer is fused to an immunoglobulin Fc polypeptide.

18. The composition of either claim 10 or claim 13 further comprising a pharmaceutically suitable excipient.

19. A fusion polypeptide comprising a poxvirus polypeptide fused with a mutein Fc polypeptide, wherein the mutein Fc polypeptide comprises the amino acid sequence of the Fc portion of a human IgG1 immunoglobulin comprising at least one mutation, wherein the at least one mutation is a substitution or a deletion of a cysteine residue in the hinge region, wherein the substituted or deleted cysteine residue is the cysteine residue most proximal to the amino terminus of the hinge region of a wildtype human IgG1 immunoglobulin Fc portion, and wherein the poxvirus polypeptide is capable of binding to a receptor-like protein tyrosine phosphatase (RPTP) selected from (i) leukocyte common antigen-related protein (LAR); (ii) RPTP-σ; and (iii) RPTP-δ.

20. The fusion polypeptide according to claim 19 wherein the mutein Fc polypeptide comprises at least one second mutation, wherein the at least one second mutation is a substitution of at least one amino acid in the CH2 domain such that the capability of the fusion polypeptide to bind to an IgG Fc receptor is reduced.

21. A composition comprising the fusion polypeptide according to claim 7 or claim 19 and a pharmaceutically suitable excipient.

22. A composition comprising (a) the antibody or antigen-binding fragment thereof, according to either claim 1 or 2, and (b) a pharmaceutically suitable excipient.

23. A composition comprising the bispecific antibody according to claim 6 and a pharmaceutically suitable excipient.

24. A method of suppressing an immune response in a subject comprising administering to the subject a composition according to claim 18.

25. A method of suppressing an immune response in a subject comprising administering to the subject a composition according to claim 21.

26. A method of suppressing an immune response in a subject comprising administering to the subject a composition according to claim 22.

27. A method of suppressing an immune response in a subject comprising administering to the subject a composition according to claim 23.

28. A method for treating an immunological disease or disorder in a subject comprising administering to the subject a composition according to claim 18.

29. A method for treating an immunological disease or disorder in a subject comprising administering to the subject a composition according to claim 21.

30. A method for treating an immunological disease or disorder in a subject comprising administering to the subject a composition according to claim 22.

31. A method for treating an immunological disease or disorder in a subject comprising administering to the subject a composition according to claim 23.

32. A method of manufacture for producing the antibody according to either claim 1 or 2.

33. A method of manufacture for producing the bispecific antibody according to claim 6.

34. A method of manufacture for producing the fusion polypeptide according to either claim 7 or 19.

35. A method of manufacture for producing the composition according to either claim 10 or claim 13.
